(19) United States
(12) Patent Application Publication
(10) Pub. No.: US 2008/0242667 A1
(43) Pub. Date: Oct. 2, 2008

(54) Pyrimidinyl-Pyrazole Inhibitors of Aurora Kinases

(75) Inventors: Jerry Leroy Adams, Collegeville, PA (US); Thomas H. Faitg, Collegeville, PA (US); Jeffrey M. Ralph, Collegeville, PA (US); Domingos J. Silva, Collegeville, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: SMITHKLINE BEECHAM CORPORATION

(21) Appl. No.: 12/064,820

(22) PCT Filed: Aug. 23, 2006
(86) PCT No.: PCT/US06/32746

§ 371 Date: Feb. 26, 2008

Related U.S. Application Data
(60) Provisional application No. 60/711,979, filed on Aug. 26, 2005.

(51) Int. Cl. A61K 31/5377; C07D 413/02; A61K 31/496; A61P 35/00; C07D 403/02

(52) U.S. Cl. 514/235.8; 544/122; 544/295; 514/252.14

(57) ABSTRACT

The present invention provides a compound represented by Formula (I):

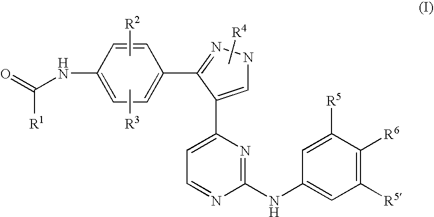

or a salt thereof, or a solvate thereof, or a combination thereof, wherein the substituents are as defined herein.

The present invention also relates to a composition comprising the compound of formula (I) and diluents, carriers, or excipients. Furthermore, the present invention relates to a method of treating a disease of cell proliferation comprising administering to a patient in need thereof a pharmaceutically effective amount of the compound of formula (I) or a salt thereof, or a solvate thereof, or a combination thereof.

### BACKGROUND OF THE INVENTION

The present invention relates to pyrimidinyl-pyrazole compounds, compositions and medicaments thereof, as well as processes for the preparation and use of such compounds, compositions and medicaments. Such pyrimidinyl-pyrazole compounds are potentially useful in the treatment of diseases associated with Aurora kinase activity.

Protein kinases catalyze the phosphorylation of hydroxylic amino acid side chains in proteins by the transfer of the γ-phosphate of ATP-Mg²⁺ to form a mono-phosphate ester of serine, threonine or tyrosine. Studies have shown that protein kinases are key regulators of many cell functions, including signal transduction, transcriptional regulation, cell motility and cell division. Several oncogenes have also been shown to encode protein kinases, suggesting that kinases may play a role in oncogenesis.

The protein kinase family of enzymes is typically classified into two main subfamilies: protein tyrosine kinases and protein serine/threonine kinases, based on the amino acid residue they phosphorylate. Aberrant protein serine/threonine kinase activity has been implicated or is suspected in a number of pathologies such as rheumatoid arthritis, psoriasis, septic shock, bone loss, cancers and other proliferative diseases. Tyrosine kinases play an equally important role in cell regulation. These kinases include several receptors for molecules such as growth factors and hormones, including epidermal growth factor receptor, insulin receptor and platelet derived growth factor receptor. Studies have indicated that many tyrosine kinases are transmembrane proteins with their receptor domains located on the outside of the cell and their kinase domains on the inside. Accordingly, both kinase subfamilies and their signal transduction pathways are important targets for drug design.

Since its discovery in 1997, the mammalian Aurora family of serine/threonine kinases has been closely linked to tumorigenesis. The three known mammalian family members, Aurora-A (“2”), B (“1”) and C (“3”), are highly homologous proteins responsible for chromosome segregation, mitotic spindle function and cytokinesis. Aurora expression is low or undetectable in resting cells, with expression and activity peaking during the G2 and mitotic phases in cycling cells. In mammalian cells, proposed substrates for the Aurora A and B kinases include histone H3, CENP-A, myosin II regulatory light chain, protein phosphatase 1, TPX2, INCENP, p53 and survivin, many of which are required for cell division.

The Aurora kinases have been reported to be over-expressed in a wide range of human tumors. Elevated expression of Aurora-A has been detected in colorectal, ovarian and pancreatic cancers, and in invasive duct adenocarcinomas of the breast. High levels of Aurora-A have also been reported in renal, cervical, neuroblastoma, melanoma, lymphoma, pancreatic and prostate tumor cell lines. Amplification/over-expression of Aurora-A is observed in human bladder cancers, and amplification of Aurora-A is associated with aneuploidy and aggressive clinical behavior. Moreover, amplification of the Aurora-A locus (20q13) correlates with poor prognosis for patients with node-negative breast cancer. In addition, an allelic variant, isoleucine at amino acid position 31, is reported to be a low-penetrance tumor-susceptibility gene. This variant displays greater transforming potential than the phenylalanine-31 variant and is associated with increased risk for advanced and metastatic disease. Like Aurora A, Aurora-B is also highly expressed in multiple human tumor cell lines, including leukemic cells. Levels of Aurora-B increase as a function of Duke's stage in primary colorectal cancers. Aurora-C, which is normally only found in germ cells, is also over-expressed in a high percentage of primary colorectal cancers and in a variety of tumor cell lines, including cervical adenocarinoma and breast carcinoma cells.

The prior art supports the hypothesis that in vitro an inhibitor of Aurora kinase activity would disrupt mitosis causing cell cycle defects and eventual cell death. Therefore, in vivo, an Aurora kinase inhibitor should slow tumor growth and induce regression. For example, Hauf et al. describe an Aurora B inhibitor, Hesperadin, that causes defects in chromosomal segregation and a block in cytokinesis, thereby resulting in polyploidy [Hauf, S et al. JCB 161(2), 281-294 (2003)]. Ditchfield et al. have described an equipotent inhibitor of Aurora A and B (ZM447439) that causes defects in chromosome alignment, chromosome segregation and cytokinesis [Ditchfield, C. et al., JCB 161(2), 267-280 (2003)]. Furthermore, the authors show that proliferating cells, but not cell-cycle arrested cells, are sensitive to the inhibitor. Efficacy of a potent Aurora A and B inhibitor in mouse and rat xenograft models was recently reported [Harrington, E. A. et al., Nature Medicine 10(3), 262-267, (2004)]. These results demonstrate that inhibition of Aurora kinases can provide a therapeutic window for the treatment of proliferative disorders such as cancer (see Nature, Cancer Reviews, Vol. 4, p927-936, December 2004, for a review by N. Keen and S. Taylor, which outlines the therapeutic potential of Aurora kinase inhibitors for the treatment of cancer).

In view of the teachings of the art, there is a need for the discovery of kinase activity inhibitors, in particular, compounds that inhibit the activity of Aurora kinases.

### SUMMARY OF THE INVENTION

In a first aspect, the present invention is a compound of formula (I):

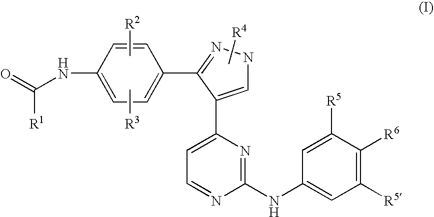

or a pharmaceutically acceptable salt thereof, or a solvate thereof, or a combination thereof, wherein:
R¹ represents phenyl, substituted phenyl, heteroaryl, C₁-C₆ alkyl, C₃-C₆ cycloalkyl, or —NR⁷R⁸;
R² and R³ each independently represent H, halo, C₁-C₃ alkyl, or —O—C₁-C₃ alkyl;
R⁴, a substituent for one of the nitrogen atoms of the pyrazole ring, represents H, C₁-C₆ alkyl, substituted C₁-C₆ alkyl, C₃-C₆ cycloalkyl, —C(O)C₁-C₆ alkyl, —C(O)-substituted C₁-C₆ alkyl, —C(O)NR⁷R⁸, —S(O)₂—C₁-C₆ alkyl, —S(O)₂—C₃-C₆ cycloalkyl, or —C(O)NH—C₁-C₆ alkyl;
R⁵, R^{5′}, and R⁶ each independently represent H, halo, C₁-C₆ alkyl, substituted C₁-C₆ alkyl, —NH—C(O)-substituted C₁-C₆ alkyl, —NR⁷R⁸, —O—C₁-C₆ alkyl, —O-substituted C₁-C₆ alkyl or hydroxyl; and
R⁷ and R⁸ each independently represent H, C₁-C₆ alkyl, substituted C₁-C₆ alkyl, C₃-C₆ cycloalkyl, phenyl, substituted phenyl or heteroaryl, or form, together with the nitrogen atom to which they are attached, a substituent selected from the group consisting of pyrrolidinyl, piperidinyl, morpholinyl, piperazinyl, 4-(C₁-C₆ alkyl)-piperazin-1-yl, and 4-(hydroxy-C₂-C₆ alkyl)-piperazin-1-yl.

In a second aspect, the present invention is a composition comprising the compound represented by Formula (I), or a salt thereof, or a solvate thereof, or a combination thereof, in admixture with one or more pharmaceutically acceptable excipients.

In a third aspect, the present invention is a method for treating a disease of cell proliferation comprising administering to a patient in need thereof a compound represented by Formula I or a salt thereof, or a solvate thereof, or a combination thereof.

In a fourth aspect the present invention is a method comprising the step of administering to a patient in need thereof an effective amount of a composition comprising (a) the compound represented by Formula (I), or a salt thereof, or a solvate thereof, or a combination thereof, and (b) at least one pharmaceutically acceptable excipient.

The present invention addresses a need in the art by providing a class of pyrimidinyl-pyrazoles inhibitors of Aurora kinase activity. Such compounds are useful in the treatment of disorders associated with inappropriate Aurora kinase family activity.

### DETAILED DESCRIPTION OF THE INVENTION

In a first aspect, the present invention relates to a compound of formula (I):

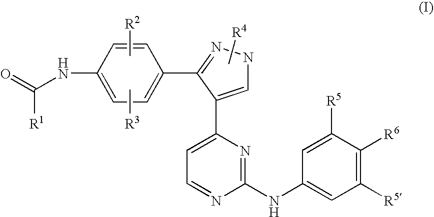

or a pharmaceutically acceptable salt thereof, or a solvate thereof, or a combination thereof, wherein:
R¹ represents phenyl, substituted phenyl, heteroaryl, C₁-C₆ alkyl, C₃-C₆ cycloalkyl, or —NR⁷R⁸;
R² and R³ each independently represent H, halo, C₁-C₃ alkyl, or —O—C₁-C₃ alkyl;
R⁴, a substituent for one of the nitrogen atoms of the pyrazole ring, represents H, C₁-C₆ alkyl, substituted C₁-C₆ alkyl, C₃-C₆ cycloalkyl, —C(O)C₁-C₆ alkyl, —C(O)-substituted C₁-C₆ alkyl, —C(O)NR⁷R⁸, —S(O)₂—C₁-C₆ alkyl, —S(O)₂—C₃-C₆ cycloalkyl, or —C(O)NH—C₁-C₆ alkyl;
R⁵, R^{5′}, and R⁶ each independently represent H, halo, C₁-C₆ alkyl, substituted C₁-C₆ alkyl, —NH—C(O)-substituted C₁-C₆ alkyl, —NR⁷R⁸, —O—C₁-C₆ alkyl, —O-substituted C₁-C₆ alkyl or hydroxyl; and
R⁷ and R⁸ each independently represent H, C₁-C₆ alkyl, substituted C₁-C₆ alkyl, C₃-C₆ cycloalkyl, phenyl, substituted phenyl or heteroaryl, or form, together with the nitrogen atom to which they are attached, a substituent selected from the group consisting of pyrrolidinyl, piperidinyl, morpholinyl, piperazinyl, 4-(C₁-C₆ alkyl)-piperazin-1-yl, and 4-(hydroxy-C₂-C₆ alkyl)-piperazin-1-yl.

As used herein, substituted phenyl refers to phenyl substituted with up to 3 groups selected from C₁-C₆-alkyl, halo, cyano, —O—C₁-C₆-alkyl, nitro, and hydroxyl.

As used herein, substituted C₁-C₆ alkyl refers to a C₁-C₆ alkyl group substituted with hydroxyl, —O—C₁-C₆ alkyl, —CO₂R⁷, —NR⁷R⁸, —C(O)NR⁷R⁸, —S(O)₂—C₁-C₆ alkyl, —S(O)ₓNR⁷R⁸ (where x is 0, 1, or 2); or up to 3 halo groups. An example of —NH—C(O)-substituted C₁-C₆ alkyl is (dimethylamino)methylcarbonylamino. Examples of substituted C₁-C₆ alkyl-NR⁷R⁸ include —(CH₂)ₙ-morpholinyl, —(CH₂)ₙ-piperidinyl, —(CH₂)ₙ-[4-(C₁-C₆ alkyl)-piperazin-1-yl], or —(CH₂)ₙ-[4-(hydroxy-C₁-C₆ alkyl)-piperazin-1-yl], where n is an integer from 1 to 6.

As used herein, heteroaryl refers to furanyl, thienyl, pyridinyl, pyrazolyl, tetrazolyl, oxazolyl, isoxazolyl, imidazolyl and pyrrolyl.

It will be understood that compounds of formula (I) may exist in alternative tautomeric form, for example when R⁴ represents a non-hydrogen substituent on the nitrogen atom in the 1-position.

Representative C₁-C₆ alkyl groups include methyl, ethyl, n-propyl, isopropyl, isobutyl, n-butyl, t-butyl, n-pentyl, and n-hexyl. Representative halo groups include fluoro, chloro, bromo and iodo groups. Examples of suitable O—C₁-C₆ alkyl groups include methoxy, ethoxy, n-propoxy, isopropoxy, n-butoxy, isobutoxy, and t-butoxy.

Representative C₃-C₆-cycloalkyl groups include cyclopropyl, cyclopentyl, and cyclohexyl groups, which may optionally be substituted with one or more C₁-C₆ alkyl groups.

As used herein, pharmaceutically acceptable refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio. The skilled artisan will appreciate that pharmaceutically acceptable salts of the compounds according to Formula (I) may be prepared. These pharmaceutically acceptable salts may be prepared in situ during the final isolation and purification of the compound, or by separately reacting the purified compound in its free acid or free base form with a suitable base or acid, respectively.

In certain embodiments, compounds according to Formula (I) may contain an acidic functional group and are, therefore, capable of forming pharmaceutically acceptable base addition salts by treatment with a suitable base. Examples of such bases include (a) hydroxides, carbonates, and bicarbonates of sodium, potassium, lithium, calcium, magnesium, aluminum, and zinc; and (b) primary, secondary, and tertiary amines including aliphatic amines, aromatic amines, aliphatic diamines, and hydroxy alkylamines such as methylamine, ethylamine, 2-hydroxyethylamine, diethylamine, triethylamine, ethylenediamine, ethanolamine, diethanolamine, and cyclohexylamine.

In certain embodiments, compounds according to Formula (I) may contain a basic functional group and are therefore capable of forming pharmaceutically acceptable acid addition salts by treatment with a suitable acid. Suitable acids include pharmaceutically acceptable inorganic acids and organic acids. Representative-pharmaceutically acceptable acids include hydrogen chloride, hydrogen bromide, nitric acid, sulfuric acid, sulfonic acid, phosphoric acid, acetic acid, hydroxyacetic acid, phenylacetic acid, propionic acid, butyric acid, valeric acid, maleic acid, acrylic acid, fumaric acid, malic acid, malonic acid, tartaric acid, citric acid, salicylic acid, benzoic acid, tannic acid, formic acid, stearic acid, lactic acid, ascorbic acid, p-toluenesulfonic acid, oleic acid, lauric acid, and the like.

As used herein, the term “a compound of Formula (I)” or “the compound of Formula (I)” refers to one or more compounds according to Formula (I). The compound of Formula (I) may exist in solid or liquid form. In the solid state, it may exist in crystalline or noncrystalline form, or as a mixture thereof. The skilled artisan will appreciate that pharmaceutically acceptable solvates may be formed for crystalline compounds wherein solvent molecules are incorporated into the crystalline lattice during crystallization. Solvates may involve non-aqueous solvents such as, but not limited to, ethanol, isopropanol, DMSO, acetic acid, ethanolamine, and ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The invention includes all such solvates.

Compounds of formula (I) may be prepared using the methods described below. In all of the schemes described below, it is understood that protecting groups may be employed where necessary in accordance with general principles known to those of skill in the art, for example, see T. W. Green and P. G. M. Wuts (1991) Protecting Groups in Organic Synthesis, John Wiley & Sons. These groups may be removed at a convenient stage of the compound synthesis using methods known to those of skill in the art. The selection of processes as well as the reaction conditions and order of their execution shall be consistent with the preparation of compounds of formula (I).

Compounds of formula (I) may be conveniently prepared by the methods outlined in Scheme 1 below. Compounds of formula (II) and (III) are commercially available or may be synthesized using techniques conventional in the art. R⁹ represents NO₂, a protected amino group (such as, but not limited to, tert-butoxycarbonylamino, cyclopropylcarbonylamino and benzoylamino group) or a group readily converted to an amino group or a protected amino group (such as a halogen or a triflate group). R¹⁰ and R¹¹ independently represent alkyl or aryl groups. Reaction of a compound of formula (II) with a compound of formula (III) yields a compound of formula (IV). This reaction may be performed using a base such as lithium hexamethyldisilazide, in an inert solvent, such as tetrahydrofuran, at low temperature, followed by quenching with an appropriate acid, such as aqueous hydrochloric acid.

The compound of formula (IV) may then be converted to a compound of formula (V) by treatment with a dialkyl acetal of dimethylformamide or an equivalent chemical entity, followed by reaction with aqueous hydrazine in a solvent such as ethanol. The compound of formula (V) may then be oxidized to a compound of formula (VI), which constitutes a sulfoxide when m=1 or a sulfone when m=2, using an oxidant such as Oxone® or meta-chloroperbenzoic acid in an appropriate solvent such as methylene chloride, tetrahydrofuran, water or methanol. The compound of formula (VI) may then be reacted with R⁴X (wherein X represents a leaving group such as, but not restricted to, halide, trifluorosulfonate, mesylate or tosylate) to afford a compound of formula (VII). This reaction may be performed in the presence of base, such as potassium t-butoxide or potassium carbonate, in a solvent, such as tetrahydrofuran, acetone or dimethylformamide, under an inert atmosphere.

Depending on the nature of the alkylating agent and the reaction conditions, the compound of formula (VII) may be isolated as a pure regioisomer or a mixture of the two possible regioisomers (where the R⁴ group is attached to one of the N atoms of the pyrazole ring). In the case where a mixture of regioisomers is obtained, these isomers may be separated by physical methods (such as crystallization or chromatographic methods) at this stage or any other later stage in the synthetic scheme.

The compound of formula (VII) may then be converted to a compound of formula (IX) by reaction with the appropriate aniline of formula (VIII), which is commercially available or may be synthesized using techniques conventional in the art. This conversion may be achieved under acidic conditions (such as, but not restricted to, heating with trifluoroacetic acid or aqueous hydrochloric acid in a solvent such as isopropanol or n-butanol) or basic conditions (such as, but not restricted to, treatment with sodium hexamethyldisilazide in tetrahydrofuran at low temperature).

In the case where R⁹ is chosen as the desired R¹C(O)NH— group, the compound of formula (IX) is indeed identical to the desired final compound of formula (I). If that is not the case, the compound of formula (IX) may be converted to a compound of formula (X), where the unmasking of the amino group is performed using methods consistent with the chemical nature of group R⁹. In the case where R⁹ is a nitro group, unmasking of the amino group may be achieved by standard reductive methods, such as, but not restricted to, hydrogenation over a reactive catalyst (such as platinum dioxide, platinum on carbon, or palladium on carbon) or reaction with stannous chloride or iron in the presence of acid. In the case where R⁹ is the tert-butylcarbonylamino group, unmasking of the amino group may be achieved by acid treatment, such as, but not restricted to, trifluoacetic acid in methylene chloride, trifluoacetic acid in water or aqueous hydrochloric acid. Those skilled in the art should recognize that other R⁹ groups may be used in this preparation, and their deprotection or conversion to the amino group should be performed according to their specific chemical nature.

The desired compound (I) may then be prepared by converting the compound of formula (X) to an amide or a urea. Amide formation may be achieved by treating the compound of formula (X) with acylating reagents such as, but are not restricted to, acyl chlorides, acid anhydrides and carboxylic acids activated by a coupling agent such as, but not limited to, HATU, HBTU or TBTU. Urea formation may be achieved, for example, (a) by treatment of the compound of formula (X) with an isocyanate in an inert solvent, or (b) by treatment of the compound of formula (X) with phosgene or equivalent in an inert solvent, followed by incubation with the amine of interest, or (c) by treatment of the amine of interest with phosgene or equivalent in an inert solvent, followed by incubation with the compound of formula (X).

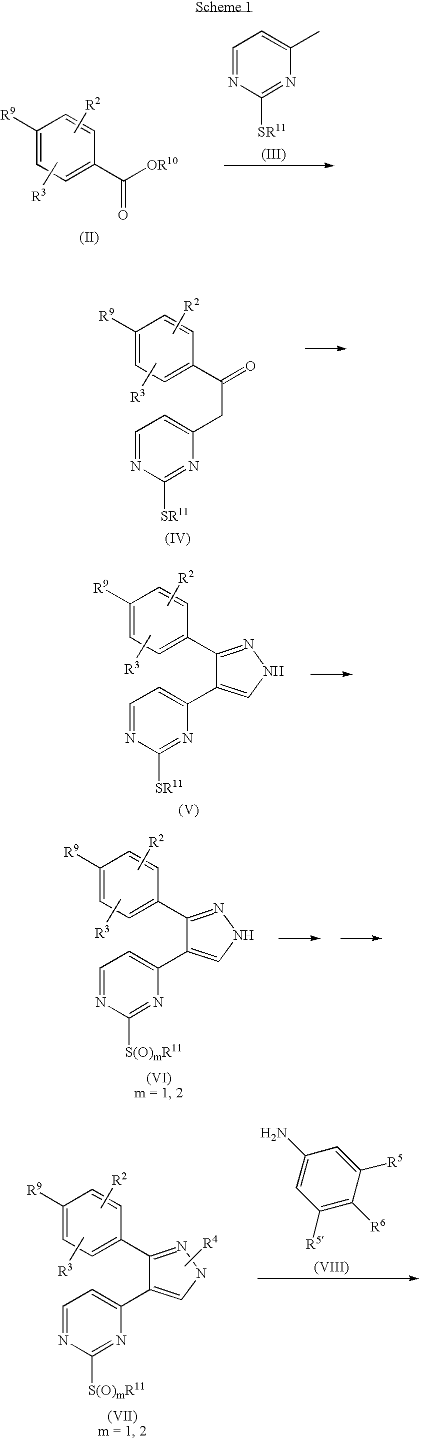

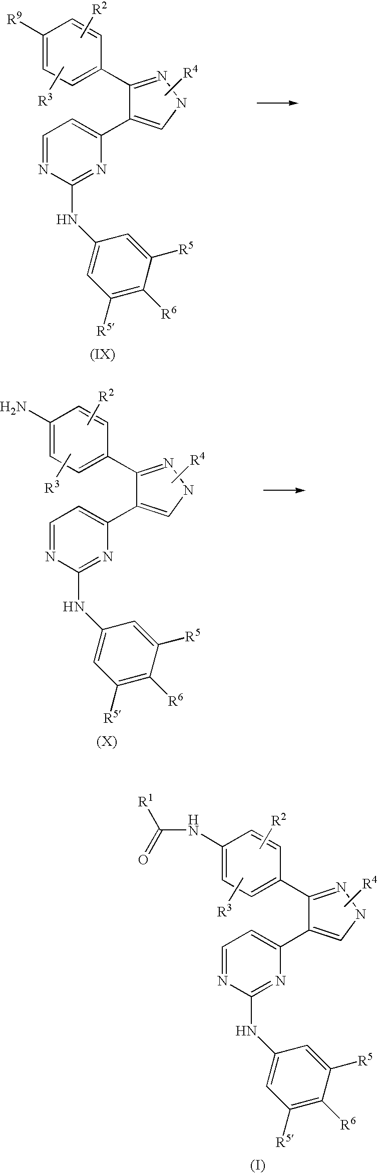

The compound of formula (V) may be also converted to the compound of formula (IX) according to the two alternative reaction sequences outlined in Scheme 2. The compound of formula (V) may be treated with strong aqueous acid, such as concentrated HCl, to yield the compound of formula (XI), which may then be converted to the compound of formula (XII) by treatment with a chlorinating agent, such as phosphorous oxychloride. The compound of formula (XII) may then be reacted with the aniline of formula (VIII), which is commercially available or may be synthesized using techniques conventional in the art. This conversion may be achieved under acidic conditions (such as, but not restricted to, heating with trifluoroacetic acid or aqueous hydrochloric acid in a solvent such as isopropanol or n-butanol) or basic conditions (such as, but not restricted to, treatment with sodium hexamethyldisilazide in tetrahydrofuran at low temperature). The compound of formula (XIII) may then be reacted with R⁴X (wherein X represents a leaving group such as, but not restricted to, halide, trifluorosulfonate, mesylate or tosylate) to afford a compound of formula (IX). This reaction may be performed in the presence of base, such as potassium t-butoxide or potassium carbonate, in a solvent, such as tetrahydrofuran, acetone or dimethylformamide, under an inert atmosphere. Alternatively, the compound of formula (V) may be alkylated with R⁴X to generate the compound of formula (XIV). Treatment of the compound of formula (XIV) with a strong aqueous acid, such as concentrated HCl, should yield the compound of formula (XV), which can be converted to the chloride (XVI) by treatment with phosphorous oxychloride. The compound of formula (XVI) may then be reacted with the aniline (VIII) under conditions described above, generating the compound of formula (IX).

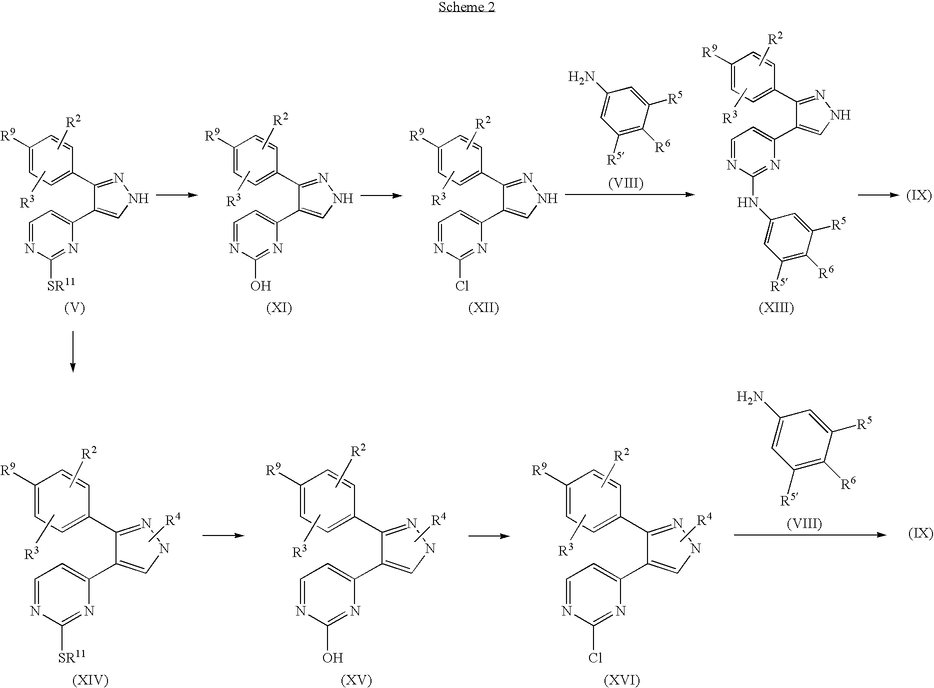

Alternatively, the compound of formula (VII) may be prepared by the route outlined on Scheme 3, where R⁴ is attached to the specified N atom of the pyrazole ring shown in that Scheme. Treatment of the compound of formula (IV) with the hydrazine R⁴NHNH₂ (which is commercially available or may be synthesized using techniques conventional in the art) yields a compound of formula (XVII). The compound of formula (XVII) may then be reacted with the dialkyl acetal of dimethylformamide or equivalent chemical entity to generate a compound of formula (XVIII). Treatment of the compound of formula (XVIII) with an oxidant, such as, but not limited to, Oxone® or meta-chloroperbenzoic acid, in an inert solvent such as methylene chloride, affords the compound of formula (VII) where the R⁴ group is attached to the specified N atom of the pyrazole ring. The compound of formula (VII) may then be converted to the compound of formula (I), where the R⁴ group is attached to the specified N atom of the pyrazole ring, according to the procedure outlined on Scheme 1.

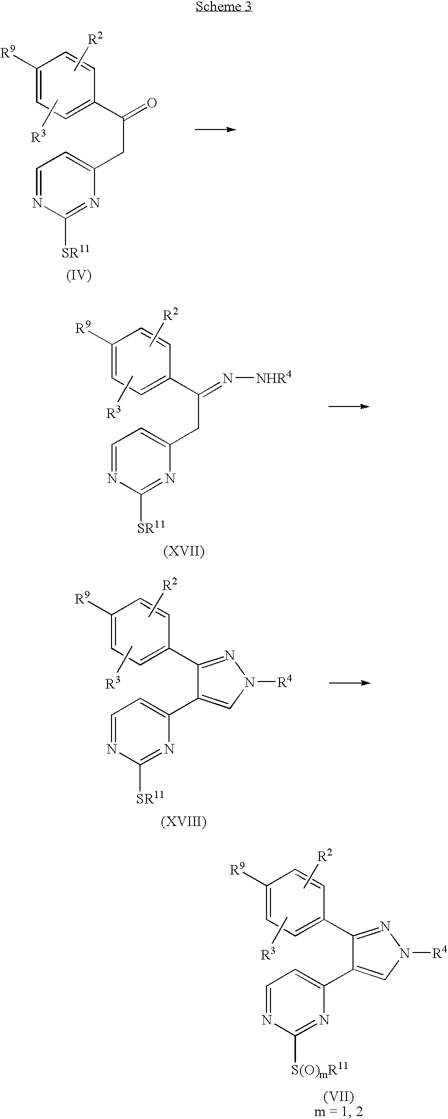

Alternatively, the compound of formula (IX) may be generated according to the reactions outlined in Scheme 4. The compound of formula (II) may be reacted with the compound of formula (XIX), which is commercially available or may be synthesized using techniques conventional in the art, to afford a compound of formula (XX). The compound of formula (XX) may then be converted to a compound of formula (XXI) by reaction with the appropriate aniline of formula (VIII), which is commercially available or may be synthesized using techniques conventional in the art. This conversion may be achieved under acidic conditions (such as, but not restricted to, heating with trifluoroacetic acid or aqueous hydrochloric acid in a solvent such as isopropanol or n-butanol) or basic conditions (such as, but not restricted to, treatment with sodium hexamethyldisilazide in tetrahydrofuran at low temperature). The compound of formula (XXI) may then be converted to a compound of formula (XXII) by treatment with a dialkyl acetal of dimethylformamide or an equivalent chemical entity, followed by reaction with hydrazine in aqueous ethanol. The compound of formula (XXII) may then be reacted with R⁴X (wherein X represents a leaving group such as, but not restricted to, halide, trifluorosulfonate, mesylate or tosylate) to afford the compound of formula (IX). This reaction may be performed in the presence of base, such as potassium t-butoxide or potassium carbonate, in an inert solvent, such as tetrahydrofuran or dimethylformamide, under an inert atmosphere. Depending on the nature of the alkylating agent and the reaction conditions, the compound of formula (IX) may be isolated as a pure regioisomer or a mixture of the two possible regioisomers (where the R⁴ group is on either N atom of the pyrazole ring). In the case where a mixture of regioisomers is obtained, these isomers may be separated by physical methods (such as crystallization or chromatographic methods) at this stage or any other later stage in the synthetic scheme. The compound of formula (IX) may be converted to the compound of formula (I) according to the procedures outlined in Scheme 1.

Alternatively, treating the compound of formula (XXI) with the hydrazine R⁴NHNH₂ (which is commercially available or may be synthesized using techniques conventional in the art) yields a compound of formula (XXIII), which may then be reacted with the dialkyl acetal of dimethylformamide or equivalent chemical entity to generate the compound of formula (IX), where the R⁴ group is attached to the specified N atom of the pyrazole ring. The compound of formula (IX) may then be converted to the compound of formula (I), where the R⁴ group is attached to the specified N atom of the pyrazole ring, according to the procedure outlined on Scheme 1.

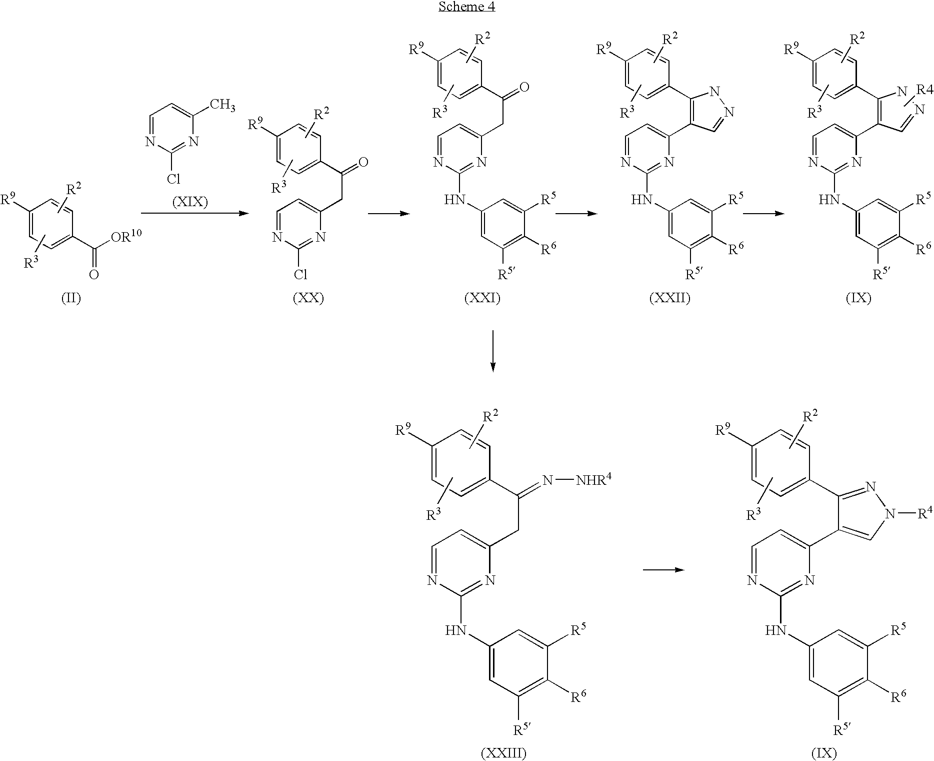

Alternatively, the compound of formula (IX) may be synthesized as shown in Scheme 5. The compound of formula (XXIV), which may be commercially available or prepared according to procedures familiar to those skilled in the art, may be reacted with a solution of DMA in DMF, followed by treatment with hydrazine, to afford the compound of formula (XXV) The compound of formula (XXV) may be reacted with the alkylating agent R⁴X (wherein X represents a leaving group such as, but not restricted to, halide, trifluorosulfonate, mesylate or tosylate) to afford the compound of formula (XXVI). This reaction may be performed in the presence of base, such as sodium hydride, cesium carbonate, potassium t-butoxide or potassium carbonate, in an inert solvent, such as tetrahydrofuran or dimethylformamide, under an inert atmosphere. The compound of formula (XXVI) may then be reacted with a brominating agent, such as NBS in DMF or bromine in chloroform, to yield the compound of formula (XXVII). The compound of formula (XXV) may also be first brominated, using a brominating agent such as NBS in DMF, and then alkylated with the alkylating agent R⁴X in the presence of a base in an inert solvent, to generate the compound of formula (XXVII). The compound of formula (XXVII) may then be submitted to standard borylation conditions (such as diborondipinacolate in the presence of a catalyst, such as palladium (II) dichloride bis(triphenylphosphine), and a base, such as potassium acetate, in an inert solvent, such as dioxane), to yield the compound of formula (XXVIII). The compound of formula (XXVIII) may then be reacted with 2,4-dichloropyrimidine, in a solvent such as methanol or ethanol, in the presence of a base such as sodium carbonate and a catalyst such as palladium (II) dichloride bis(triphenylphosphine), to afford the compound of formula (XXIX). The compound of formula (XXIX) may finally be reacted with the aniline of formula (VIII) in the presence of acid to afford the compound of formula (IX).

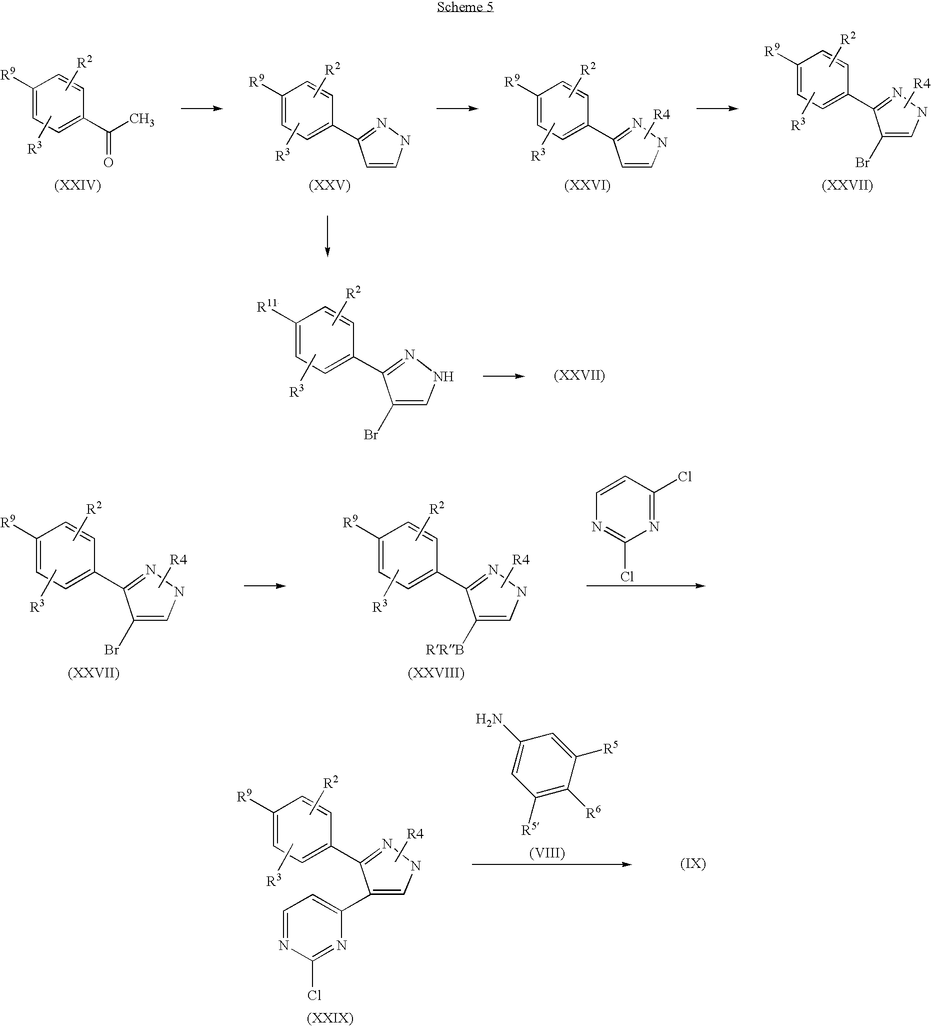

Alternatively, the compound of compound (IX) may be synthesized as shown in Scheme 6. The commercially available 4-thiouracyl (XXX) may be alkylated to afford the compound of formula (XXXI). This compound may be treated with phosphorus oxybromide to afford the bromide of formula (XXXII), which may be oxidized, using a reagent such as mCPBA, to the corresponding sulfone of formula (XXXIII). The sulfone (XXXIII) may be reacted with the aniline of formula (VIII), in the presence of a strong base such as sodium hexamethyldisilazide, to afford the compound of formula (XXXIV). A Suzuki coupling between compound (XXXIV) and compound (XXVIII), using palladium dichloride bis(triphenylphosphine) as the catalyst, maybe be used to generate the compound of formula (IX).

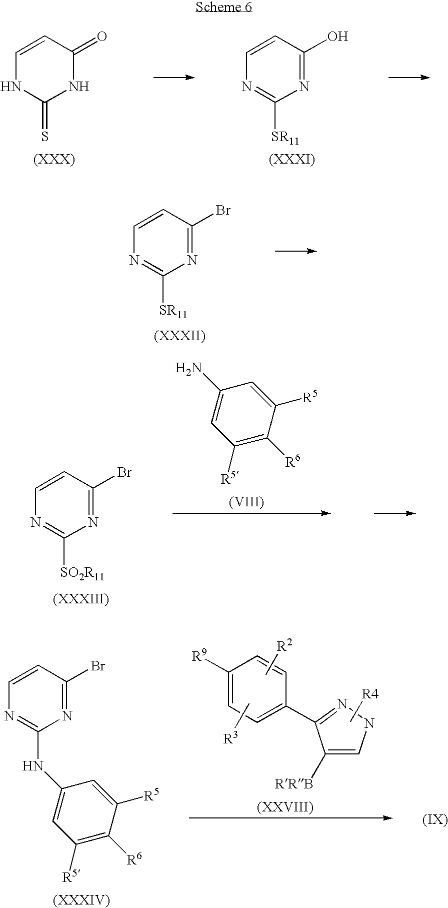

Alternatively, the compound of formula (IX) may also be generated according the reactions displayed in Scheme 7. The compound of formula (XXXIII) may be reacted with the aniline of formula (VIII) or its Boc protected version of formula (XXXV), in the presence of a base such as an alkaline hexamethyldisilazide, in an inert solvent such as THF, to afford the compound of formula (XXXVI). The compound of formula (XXXVI) may be converted to the boronate of formula (XXXVII), which may be coupled to the bromide of formula (XXVII) to afford the compound of formula (XXXVIII) when R′″ is Boc or the compound of formula (IX) when R′″ is H. The compound of formula (XXXVIII), where R′″ is Boc, may be converted to the compound of formula (IX) by acidic deprotection using, for example, hydrochloric or trifluoroacetic acid. The compound of formula (IX) may then be used to generate compound of formula (I) according to the transformations described in Scheme 1.

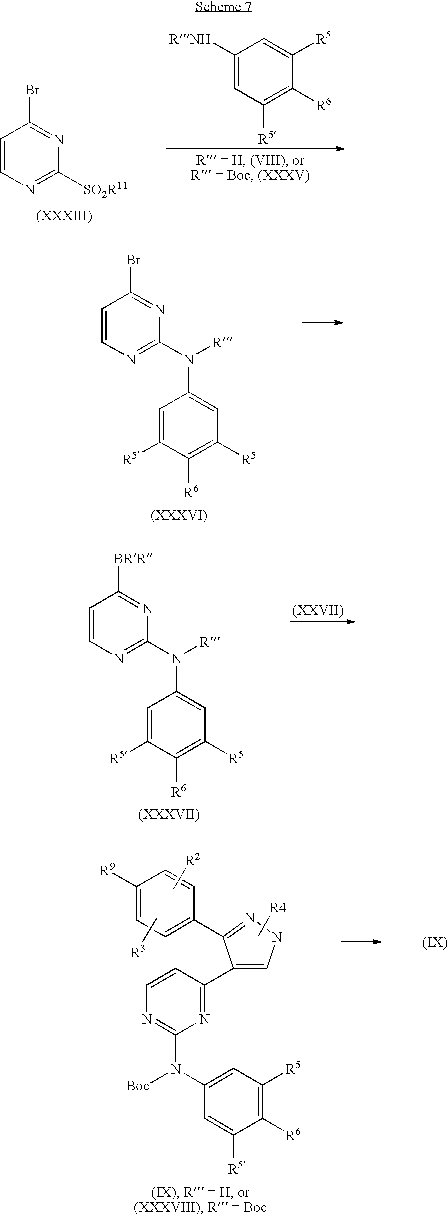

Alternatively, the compound of formula (IX) may also be generated according the reactions displayed in Scheme 8. The compound of formula (XXVI) may be converted to the iodide of formula (XXXIX) by reaction with N-iodo-succinimide, for example. The compound of formula (XXXIX) may also be prepared by conversion of compound (XXV) to the iodide of formula (XXXX) using N-iodo-succinimide, followed by alkylation with R⁴X. The compound of formula (XXXIX) may then be converted to the acetyl compound of formula (XXXXI) by treatment with trimethylsilylacetylene, copper (I) iodide, triethylamine and bis(triphenylphosphine)palladium (II) dichloride in toluene, followed by acidic hydrolysis, using conditions such as trifluoroacetic acid in a mixture of water and methylene chloride. The compound of formula (XXXXI) may then be converted to the compound of formula (XXXXII) by treatment with dimethylformamide di-t-butyl acetal. In parallel, the aniline of formula (VIII) may be converted to the guanidine of formula (XXXXIII) by initial treatment with N,N′-bis-t-butoxycarbonyl-1H-pyrazole-1-carboxamidine, followed by acidic treatment with trifluoroacetic acid or hydrochloric acid. This transformation may also be performed in a single step using 1H-pyrazole-1-carboxamidine. The compound of formula (XXXXIII) may then be reacted with the compound of formula (XXXXII) at elevated temperature (such as 125° C.) in an inert solvent, such as dimethylformamide, to afford the compound of formula (IX).

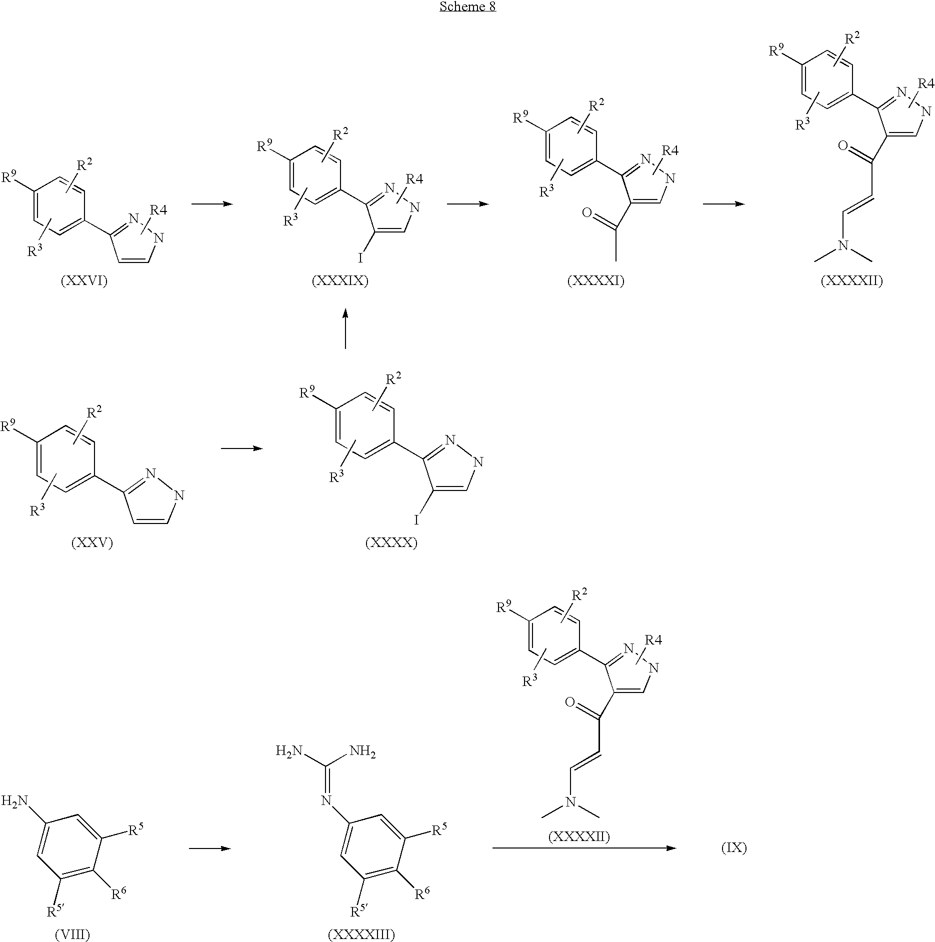

### Methods of Use

The compounds of the invention can be used to treat diseases of cellular proliferation, autoimmunity or inflammation. Disease states which can be treated by the compounds of the invention include, but are not limited to, cancer, autoimmune disease, fungal disorders, arthritis, graft rejection, inflammatory bowel disease, proliferation induced after medical procedures, including, but not limited to, surgery, angioplasty and the like (see below for further discussion of selected disease states). It is appreciated that in some cases the cells may not be in a hyper- or hypoproliferation state (abnormal state) and still require treatment. Thus, in certain embodiments, the invention includes application to cells or individuals afflicted or impending affliction with any one of these disorders or states.

### Proliferative Disease/Cancer

The present invention is directed to a class of novel kinase inhibitors, particularly inhibitors of Aurora (A, B and/or C) kinase. The present invention makes use of the finding that Aurora kinase serves multiple essential functions required for the completion of mitosis and that inhibition of the kinase activity of Aurora frequently results in cell cycle arrest and/or abnormal cell division, both of which can trigger cell death. Thus, by inhibiting Aurora kinase, cellular proliferation is blocked.

The compounds of the invention find use in a variety of applications. As will be appreciated by those skilled in the art, mitosis may be altered in a variety of ways; that is, mitosis can be affected either by increasing or decreasing the activity of a component in the mitotic pathway. Stated differently, mitosis may be disrupted by disturbing equilibrium, either by inhibiting or activating certain components. Similar approaches may be used to alter meiosis.

The compounds of the invention provided herein are particularly deemed useful for the treatment of cancer including solid tumors, such as skin, breast, brain, cervical carcinomas, testicular carcinomas and others. More particularly, cancers that may be treated using the compounds of the invention include, but are not limited to: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord (neurofibroma, meningioma, glioma, sarcoma); Gynecological: uterus (endometrial carcinoma), cervix (cervical carcinoma, pre-tumor cervical dysplasia), ovaries (ovarian carcinoma, serous cystadenocarcinoma, mucinous cystadenocarcinoma, unclassified carcinoma), granulosa-thecal cell tumors, Sertoli-Leydig cell tumors, dysgerminoma, malignant teratoma, vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma)), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Karposi's sarcoma, moles dysplastic nevi, lipoma, angioma, dermatofibroma, keloids, psoriasis; and Adrenal glands: neuroblastoma. Thus, the term “cancerous cell” as provided herein, includes a cell afflicted by any one of the above identified conditions.

Accordingly, the compounds of the invention are administered to cells. By “administered” herein is meant administration of a therapeutically effective dose of a compound of the invention to a cell either in cell culture or in a patient. By “therapeutically effective dose” herein is meant a dose that produces the effects for which it is administered. The exact dose will depend on the purpose of the treatment, and will be ascertainable by one skilled in the art using known techniques. As is known in the art, adjustments for systemic versus localized delivery, age, body weight, general health, sex, diet, time of administration, drug interaction and the severity of the condition may be necessary, and will be ascertainable with routine experimentation by those skilled in the art. By “cells” herein is meant any cell in which mitosis or meiosis can be altered. A “patient” for the purposes of the present invention includes both humans and other animals, particularly mammals, and other organisms. Thus, the methods are applicable to both human therapy and veterinary applications. In certain embodiments the patient is a mammal, especially a human.

The compounds of the invention may be administered in a physiologically acceptable carrier to a patient, as described herein. Depending upon the manner of introduction, the compounds may be formulated in a variety of ways as discussed below. The concentration of the compound in the formulation may vary from about 0.1-99.9 wt. %.

When used to treat proliferative diseases, the compounds of the present invention can be administered alone or in combination with other treatments, i.e., radiation, or other therapeutic agents, such as the taxane class of agents that appear to act on microtubule formation or the camptothecin class of topoisomerase I inhibitors. When so used, other therapeutic agents may be administered before, concurrently with (whether in separate dosage forms or in a combined dosage form) or after administration of the compound of the invention.

### Compositions

The compounds of the invention will normally, but not necessarily, be formulated into pharmaceutical compositions prior to administration to a patient. Accordingly, in another aspect the invention is directed to pharmaceutical compositions comprising a compound of the invention and one or more pharmaceutically acceptable excipient. The pharmaceutical compositions of the invention may be prepared and packaged in bulk form wherein a safe and effective amount of a compound of the invention can be extracted and then given to the patient, such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from about 0.1 to 99.9 wt. %, depending on the nature of the formulation.

As used herein, “pharmaceutically acceptable excipient” means a pharmaceutically acceptable material, composition or vehicle involved in giving form or consistency to the pharmaceutical composition. Each excipient is advantageously compatible with the other ingredients of the pharmaceutical composition when comingled, such that interactions which would substantially reduce the efficacy of the compound of the invention when administered to a patient and would result in pharmaceutically unacceptable compositions are avoided. In addition, each excipient is sufficiently high in purity to render it pharmaceutically acceptable.

The compound of the invention and the pharmaceutically acceptable excipient or excipients will typically be formulated into a dosage form adapted for administration to the patient by the desired route of administration. For example, dosage forms include those adapted for (1) oral administration, such as tablets, capsules, caplets, pills, troches, powders, syrups, elixers, suspensions, solutions, emulsions, sachets, and cachets; (2) parenteral administration, such as sterile solutions, suspensions, and powders for reconstitution; (3) transdermal administration, such as transdermal patches; (4) rectal administration, such as suppositories; (5) inhalation, such as aerosols and solutions; and (6) topical administration, such as creams, ointments, lotions, solutions, pastes, sprays, foams, and gels.

Suitable pharmaceutically acceptable excipients will vary depending upon the particular dosage form chosen. In addition, suitable pharmaceutically acceptable excipients may be chosen for a particular function that they may serve in the composition. For example, certain pharmaceutically acceptable excipients may be chosen for their ability to facilitate the production of uniform dosage forms. Certain pharmaceutically acceptable excipients may be chosen for their ability to facilitate the production of stable dosage forms. Certain pharmaceutically acceptable excipients may be chosen for their ability to facilitate the carrying or transporting the compound or compounds of the invention once administered to the patient from one organ, or portion of the body, to another organ, or portion of the body. Certain pharmaceutically acceptable excipients may be chosen for their ability to enhance patient compliance.

Suitable pharmaceutically acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically acceptable excipients in appropriate amounts for use in the invention. In addition, there are a number of resources that are available to the skilled artisan which describe pharmaceutically acceptable excipients and may be useful in selecting suitable pharmaceutically acceptable excipients. Examples include Remington's Pharmaceutical Sciences (Mack Publishing Company), Remington: The Science and Practice of Pharmacy, (Lippincott Williams & Wilkins), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

Oral solid dosage forms such as tablets will typically comprise one or more pharmaceutically acceptable excipients, which may for example help impart satisfactory processing and compression characteristics, or provide additional desirable physical characteristics to the tablet. Such pharmaceutically acceptable excipients may be selected from diluents, binders, glidants, lubricants, disintegrants, colorants, flavorants, sweetening agents, polymers, waxes or other solubility-modulating materials.

Dosage forms for parenteral administration will generally comprise fluids, particularly intravenous fluids, i.e., sterile solutions of simple chemicals such as sugars, amino acids or electrolytes, which can be easily carried by the circulatory system and assimilated. Such fluids are typically prepared with water for injection USP. Fluids used commonly for intravenous (IV) use are disclosed in Remington, The Science and Practice of Pharmacy [full citation previously provided], and include:
alcohol, e.g., 5% alcohol (e.g., in dextrose and water (“D/W”) or D/W in normal is saline solution (“NSS”), including in 5% dextrose and water (“D5/W”), or D5/W in NSS);
synthetic amino acid such as Aminosyn, FreAmine, Travasol, e.g., 3.5 or 7; 8.5; 3.5, 5.5 or 8.5% respectively;
ammonium chloride e.g., 2.14%;
dextran 40, in NSS e.g., 10% or in D5/W e.g., 10%;
dextran 70, in NSS e.g., 6% or in D5/W e.g., 6%;
dextrose (glucose, D5/W) e.g., 2.5-50%;
dextrose and sodium chloride e.g., 5-20% dextrose and 0.22-0.9% NaCl;
lactated Ringer's (Hartmann's) e.g., NaCl 0.6%, KCl 0.03%, CaCl₂ 0.02%;
lactate 0.3%;
mannitol e.g., 5%, optionally in combination with dextrose e.g., 10% or NaCl e.g., 15 or 20%;
multiple electrolyte solutions with varying combinations of electrolytes, dextrose, fructose, invert sugar Ringer's e.g., NaCl 0.86%, KCl 0.03%, CaCl₂ 0.033%;
sodium bicarbonate e.g., 5%;
sodium chloride e.g., 0.45, 0.9, 3, or 5%;
sodium lactate e.g., ⅙ M; and
sterile water for injection

The pH of such IV fluids may vary, and will typically be from 3.5 to 8 as known in the art.

It will be appreciated that when the compounds of the present invention are administered in combination with other therapeutic agents normally administered by the inhaled, intravenous, oral or intranasal route, that the resultant pharmaceutical composition may be administered by the same routes.

Compounds of the invention may conveniently be administered in amounts of, for example, 0.001 to 500 mg/kg body weight. The precise dose will of course depend on the age and condition of the patient and the particular route of administration chosen.

Compounds of the invention were tested for in vitro activity in accordance with the following assays. The following compounds have an IC₅₀ of less than 10 μM for Aurora A or Aurora B or both as determined by the following assays described.

### Aurora A Enzyme Activity Assay

Compounds of the present invention were tested for Aurora A protein kinase inhibitory activity in substrate phosphorylation assays. This assay examines the ability of small molecule organic compounds to inhibit the serine phosphorylation of a peptide substrate, and was run in the LEADseeker (Amersham Bioscience, Piscataway, N.J.) scintillation proximity assay (SPA) format.

The substrate phosphorylation assays use recombinant human full-length Aurora A kinase expressed in baculovirus/Sf9 system. A N-terminal His-Thr-affinity tag was fused to the amino terminus of amino acids 2 through 403 of Aurora A. 5 nM okadaic acid was added during the last 4 hours of expression (experimentally determined to enhance Aurora A's enzymatic activity). The enzyme was purified to approximately 70% purity by metal-chelate affinity chromatography.

The method measures the ability of the isolated enzyme to catalyze the transfer of the gamma-phosphate from ATP onto the serine residue of a biotinylated synthetic peptide (Biotin-aminohexyl-RARRRLSFFFFAKKK-amide). Substrate phosphorylation was detected by the following procedure: Assays were performed in 384-well low volume white polystyrene plates (Greiner Bio-One, Longwood, Fla.). 1 nM Aurora A enzyme was added to the wells containing 0.1 μl of test compound in 100% DMSO and incubated for 30 minutes, followed by the addition of reaction mixture resulting in a final assay volume of 10 μl containing 6 mM magnesium chloride, 1.5 μM ATP, 1 μM peptide substrate, 40 nM microtubule associated protein TPX2 peptide (1-43), 0.03 μCi [gamma-P³³] ATP/well, 5 mM DTT, 25 mM KCl, 0.15 mg/ml BSA and 0.01% Tween-20 in 50 mM HEPES, pH 7.2. The reaction was allowed to proceed for 120 minutes at room temperature and was terminated by the addition of 10 μl of a LEADseeker SPA bead solution containing PBS (Dulbecco's PBS without Mg²⁺ and Ca²⁺), 50 mM EDTA, 0.03 mg of Streptavidin coupled polystyrene imaging beads (Amersham Bioscience). The plate was sealed and the beads were allowed to incubate overnight. The plate was read in a Viewlux (Wallac, Turku, Finland) plate reader.

For dose response curves, data were normalized and expressed as % inhibition using the formula 100*(1−(U−C2)/(C1−C2)) where U is the unknown value, C1 is the average of the high signal (0% inhibition) and C2 is the average of the low signal (100% inhibition) control wells. Curve fitting was performed with the following equation: y=A+((B−A)/(1+(10̂x/10̂C)̂D)), where A is the minimum response, B is the maximum response, C is the log 10(XC50), and D is the slope. The results for each compound were recorded as pIC50 values (−C in the above equation).

### Aurora B Enzyme Activity Assay

Compounds of the present invention were tested for Aurora B protein kinase inhibitory activity in substrate phosphorylation assays. This assay examines the ability of small molecule organic compounds to inhibit the serine phosphorylation of a peptide substrate, and was run in the LEADseeker (Amersham Bioscience) scintillation proximity assay (SPA) format.

The substrate phosphorylation assays use recombinant human full-length Aurora B kinase expressed in baculovirus/Sf9 system. Following expression the culture is incubated with 50 nM okadaic acid for 1 hour prior to purification. An N-terminal His-affinity tag was fused to the amino terminus of amino acids 1 through 344 of Aurora B. 5 μM Aurora B was activated in 50 mM Tris-HCl pH 7.5, 0.1 mM EGTA, 0.1% 2-mercaptoethanol, 0.1 mM sodium vandate, 10 mM magnesium acetate, 0.1 mM ATP with 0.1 mg/ml GST-INCENP [826-919] at 30° C. for 30 mins. Following activation the enzyme is then dialysed into enzyme storage buffer and stored at −70° C.

The method measures the ability of the isolated enzyme to catalyze the transfer of the gamma-phosphate from ATP onto the serine residue of a biotinylated synthetic peptide (Biotin-aminohexyl-RARRRLSFFFFAKKK-amide). Substrate phosphorylation was detected by the following procedure: Assays were performed in 384-well low volume white polystyrene plates (Greiner Bio-One, Longwood, Fla.). 5 nM Aurora B enzyme was added to the wells containing 0.1 μl of test compound in 100% DMSO and incubated for 30 minutes followed by the addition of reaction mixture resulting in a final assay volume of 10 μl containing 6 mM magnesium chloride, 3 mM manganese chloride, 1.25 μM ATP, 1.25 μM peptide substrate, 0.025 μCi [gamma-P³³] ATP/well, 5 mM DTT, 0.15 mg/ml BSA, 0.01% Tween-20 in 50 mM HEPES pH 7.5, and 0.1 μl of test compound in 100% DMSO. The reaction was allowed to proceed for 120 minutes at room temperature and was terminated by the addition of 10 μl of a LEADseeker SPA bead solution containing PBS (Dulbecco's PBS without Mg²⁺ and Ca²⁺), 50 mM EDTA, 0.03 mg of Streptavidin coupled polystyrene imaging beads (Amersham Bioscience). The plate was sealed and the beads were allowed to incubate overnight. The plate was read in a Viewlux (Wallac, Turku, Finland) plate reader.

For dose response curves, data were normalized and expressed as % inhibition using the formula 100*(1−(U−C2)/(C1−C2)) where U is the unknown value, C1 is the average of the high signal (0% inhibition) and C2 is the average of the low signal (100% inhibition) control wells. Curve fitting was performed with the following equation: y=A+((B−A)/(1+(10̂X/10̂C)̂D)), where A is the minimum response, B is no the maximum response, C is the log 10(XC50), and D is the slope. The results for each compound were recorded as pIC50 values (−C in the above equation).

### Cellular Proliferation Assay:

The ability of compounds to inhibit the proliferation of human tumor or normal cells was investigated using cell proliferation assays. Briefly, cells are seeded into 96 well plates at an appropriate density for each cell type to ensure logarithmic growth throughout the assay and allowed to adhere overnight. Compounds are dissolved in 100% DMSO at approximately 10 mM and two-fold serially dilutions are made in 100% DMSO spanning twenty concentration points. Compounds are diluted 500-fold into cell culture media and incubated on cells for three days. Cell viability is determined using Promega's CellTiter-Glo reagent as per manufacturer's instructions. Percent growth proliferation is calculated relative to DMSO alone treated cells and IC50 values are determined by a four-parameter fit model using XIfit (IDBS, Inc.).

### General Purification and Analytical Methods

Analytical HPLC was conducted on a Zorbex Eclipse XD8-C18 column (4.6×150 mm, 5 um), using H₂O with 0.05% TFA (solvent A) and CH₃CN with 0.05% TFA (solvent B). The elution gradient was 10-90% B over 15 min; flow 1.0 mL/min. Detection: 230 and 254 nm. Retention times (t_{R}) are reported in minutes.

Preparative HPLC was conducted on a Phenomenex Gemini 5u C18 110A (100×30.0 mm, 5 μm), using H₂O with 0.1% formic acid (solvent A) and CH₃CN with 0.1% formic acid (solvent B). The isocratic elution used was 18-24% B over 8 min, then gradient ramp up to 90% B over 2 min; flow 55 mL/min. Detection: 230 or 254 nm.

LC-MS analysis was performed on a Perkin Elmer Sciex 100 atmospheric pressure ionization (APCI) mass spectrometer. Retention times in LC-MS are referred to as t_{R} (time in minutes).

¹H NMR spectra were recorded using a Bruker DPX 400 MHz spectrometer referenced to tetramethylsilane. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Analogix™ chromatography refers to purification carried out using equipment sold by Analogix Corporation (IntelliFlash 280) and cartridges PuriFlash (RS or SF) pre-packed with PuriSil. Hydrophobic filtration frits were obtained from Whatman. TLC (thin layer chromatography) plates coated with silica gel 60 F254 were obtained from Merck.

Examples or intermediates purified by preparative HPLC were obtained as the corresponding formate salt, unless specified differently.

### EXAMPLES

The following examples are for illustrative purposes only and are not intended to limit the scope of this invention. As used herein, the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. All temperatures are in ° C. Unless otherwise noted, all starting materials were obtained from commercial suppliers and used without further purification. Specifically, the following abbreviations may be used in the examples and throughout the specification:

| | |
|---|---|
| g (grams); | mg (milligrams); |
| L (liters); | mL (milliliters); |
| μL (microliters); | psi (pounds per square inch); |
| M (molar); | mM (millimolar); |
| Hz (Hertz); | MHz (megahertz); |
| mmol (millimoles); | mol (moles); |
| min (minutes); | h (hours); |
| mp (melting point); | TLC (thin layer chromatography); |

HPLC (high pressure liquid chromatography);
atm (atmosphere);

| | |
|---|---|
| t_{R} (retention time); | RP (reverse phase); |
| MeOH (methanol); | i-PrOH (isopropanol); |
| TEA (triethylamine); | TFA (trifluoroacetic acid); |
| THF (tetrahydrofuran); | DMSO (dimethylsulfoxide); |
| AcOEt (EtOAc); | DCM (CH2Cl2); |
| DMF (N,N-dimethylformamide); | |
| HOAc (acetic acid); | mCPBA (meta-chloroperbenzoic acid); |
| BOC (tert-butyloxycarbonyl); | Ac (acetyl); |

DMAP (4-dimethylaminopyridine)

| | |
|---|---|
| ATP (adenosine triphosphate); | BSA (bovine serum albumin) |

HBTU (O-Benzotriazole-1-yl-N,N,N′,N′-tetramethyluronium
hexafluorophosphate);
HEPES (4-(2-hydroxyethyl)-1-piperazine ethane sulfonic acid);
DMF (N,N-dimethylformamide);
NaHMDS (sodium hexamethyldisilazide)
DMF-DMA (N,N-dimethylformamide dimethylacetal).

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted.

### Intermediate 1

### 4-Methyl-2-(methylthio)pyrimidine

A suspension of 2-thio-4-methylpyrimidine (20.0 g) and methyl iodide (7.65 g) in ethanol (615 mL) and 1M NaOH (246 mL) was stirred at room temperature for 16 h. The reaction mixture was concentrated under reduced pressure to 200 mL, and then the reaction mixture was extracted with ethyl acetate (300 mL×2). The organic layers were combined and washed with water, brine, and dried with anhydrous Na₂SO₄. The mixture was filtered and concentrated to afford the title compound as clear brown oil.

### Intermediate 2

### Ethyl 4-[(phenylcarbonyl)amino]benzoate

A suspension of ethyl 4-aminobenzate (10.0 g) in methylene chloride (250 mL) and triethylamine (17.5 mL) was treated with benzoyl chloride at 0° C., allowed to warm to room temperature and stirred for 16 h. The reaction mixture was diluted with water (300 mL) and extracted with methylene chloride (200 mL×2). The organic layers were washed with water, brine, and dried with anhydrous Na₂SO₄. The mixture was filtered and concentrated. The residue was dissolved in hot diethyl ether, then cooled to 0° C. The title compound was isolated after filtration of the diethyl ether solution. ¹H NMR (400 MHz, CDCl₃) δ ppm 8.09 (d, J=8.8 Hz, 2H), 7.98 (s, 1H) 7.94-7.89 (m, 2H), 7.77 (d, J=8.8 Hz, 2H), 7.64-7.60 (m, 1H), 7.57-7.52 (m, 2H), 4.40 (q, J=7.1 Hz, 2H), 1.42 (t, J=7.2 Hz, 3H); ESI MS (m/z) 270 [M+H]⁺.

### Intermediate 3

### N-(4-{2-[2-(Methylthio)-4-pyrimidinyl]acetyl}phenyl)benzamide

A suspension of 4-methyl-2-(methylthio)pyrimidine (3.5 g) and ethyl 4-[(phenylcarbonyl)amino]benzoate (6.72 g) in THF (160 mL) was treated with 1M lithium bis(trimethylsiyl)amide solution in THF (80 mL) at −78° C. The reaction mixture was warmed to 0° C. over the period of 3 h. The reaction mixture was then poured into a 1:1 mixture of 1M hydrochloric acid/ice (80 mL each) and stirred for 2 h. The reaction mixture was filtered to afford the title compound as a yellow solid. ESI MS (m/z) 364 [M+H]⁺; LC-MS, t_{R} (enol)=2.10 min, t_{R} (ketone)=2.52 min.

### Intermediate 4

### N-(4-{4-[2-(Methylthio)-4-pyrimidinyl]-1H-pyrazol-3-yl}phenylbenzamide

A suspension of N-(4-{2-[2-(methylthio)-4-pyrimidinyl]acetyl}phenyl)benzamide (5.0 g) in N,N-dimethylformamide dimethyl acetal (36 mL) was heated to 100° C. for 3 h. The solvent was then removed under reduced pressure. The crude residue was dissolved in ethanol (40 mL), and treated with 35 wt % hydrazine solution in water (9.96 mL) at 0° C. for 3 h. The solvent was removed under reduced pressure. The residue was washed with hot methylene chloride. The title compound was isolated after filtration of the methylene chloride solution. ¹H NMR (400 MHz, CDCl₃) δ ppm 10.55 (m, 1H), 8.33 (d, J=5.3 Hz, 1H), 8.24 (s, 2H), 8.01 (s, 1H), 7.91-7.96 (m, 2H), 7.78 (d, J=8.6 Hz, 2H), 7.63-7.52 (m, 4H), 6.88 (d, J=5.3 Hz, 1H), 2.50 (s, 3H); ESI MS (m/z) 388 [M+H]⁺; analytical HPLC t_{R}=5.67 min.

### Intermediate 5

### N-(4-{4-[2-(Methylsulfonyl)-4-pyrimidinyl]-1H-pyrazol-3-yl}phenylbenzamide

A suspension of N-(4-{4-[2-(methylthio)-4-pyrimidinyl]-1H-pyrazol-3-yl}phenyl)benzamide (489 mg) in methylene chloride (12 mL) was treated with 3-chloroperoxy benzoic acid (849 mg) at 0° C. and then warmed to room temperature. After 3 h, the reaction was diluted with water (40 mL) and extracted with methylene chloride (4×20 mL). The methylene chloride layers were concentrated. The title compound was isolated by purification of this residue by pad of Silica gel using ethyl acetate:hexanes (1:3) as eluent. ¹H NMR (400 MHz, DMSO-d₆) δ ppm 13.60 (s, 1H), 10.43-10.32 (m, 1H), 8.85 (d, J=5.3 Hz, 2H), 8.67 (d, J=1.0 Hz, 1H), 8.31 (d, J=1.5 Hz, 1H), 7.76-7.69 (m, 3H), 7.69-7.63 (m, 2H), 7.55 (d, J=8.8 Hz, 2H), 7.50 (d, J=8.6 Hz, 1H), 3.18-3.15 (m, 3H); ESI MS (m/z) 420 [M+H]⁺.

### Example 1

### N-(4-{4-[2-({3-[2-(4-Morpholinyl)ethyl]phenyl}amino)-4-pyrimidinyl]-1H-pyrazol-3-yl}phenyl)benzamide

A suspension of N-(4-{4-[2-(methylsulfonyl)-4-pyrimidinyl]-1H-pyrazol-3-yl}phenyl)benzamide (51 mg) and 3-[2-(4-morpholinyl)ethyl]aniline (30 mg) in THF (3 mL) was treated with 1M sodium bis(trimethylsilyl)amide solution in THF at −78° C. The reaction mixture was warmed to 0° C. for 1 h. The reaction mixture was diluted with saturated aqueous sodium bicarbonate (5 mL), extracted with ethyl acetate (3×8 mL), and dried with Na₂SO₄. The combined organic layers were filtered and concentrated. The residue was dissolved with hot methylene chloride, diluted with hexanes, and cooled to 0° C. The title compound was isolated after filtration of the methylene chloride:hexanes solution. ¹H NMR (400 MHz, DMSO-d₆) δ ppm 13.41-13.34 (m, 1H), 10.44-10.35 (m, 1H), 9.42 (d, J=5.3 Hz, 1H), 8.30 (d, J=5.6 Hz, 1H), 8.07-7.84 (m, 4H), 7.63-7.54 (m, 6H), 7.46 (d, J=8.1 Hz, 1H), 7.09-7.06 (m, 1H), 6.75 (d, J=7.3 Hz, 1H), 6.66 (d, J=5.0 Hz, 1H), 3.59-3.56 (m, 4H), 2.69-2.63 (m, 2H), 2.54-2.50 (m, 2H), 2.44-2.39 (m, 4H); ESI MS (m/z) 546 [M+H]⁺; analytical HPLC t_{R}=4.32 min.

### Intermediate 6

### Mixture of N-(4-{1-methyl-4-[2-(methylsulfonyl)-4-pyrimidinyl]-1H-pyrazol-3-yl}phenyl)benzamide & N-(4-{1-Methyl-4-[2-(methylsulfonyl)-4-pyrimidinyl]-1H-pyrazol-5-yl}phenyl)benzamide

A suspension of N-(4-{4-[2-(methylsulfonyl)-4-pyrimidinyl]-1H-pyrazol-3-yl}phenyl)benzamide (419 mg) in DMF (10 mL) was treated with potassium tert-butoxide (136 mg) and methyl iodide (71 μL) at 0° C. and then warmed to room temperature. After 2 h, the reaction mixture was diluted with saturated aqueous sodium bicarbonate (5 mL) and extracted with ethyl acetate (3×8 mL). The ethyl acetate layers were concentrated. The residue was purified by flash chromatography to give the title compounds as a white solid. ESI MS (m/z) 434 [M+H]⁺; HPLC t_{R}=5.29, 5.39 min.

### Examples 2 and 3

A suspension of N-(4-{1-methyl-4-[2-(methylsulfonyl)-4-pyrimidinyl]-1H-pyrazol-3-yl}phenyl)benzamide and N-(4-{1-methyl-4-[2-(methylsulfonyl)-4-pyrimidinyl]-1H-pyrazol-5-yl}phenyl)benzamide (140 mg, 1:1 mixture) and 3-[2-(4-morpholinyl)ethyl]aniline (80 mg) in THF (8 mL) was treated with 1M sodium bis(trimethylsilyl)amide solution in THF (1.61 mL) at −78° C. The reaction mixture was warmed to 0° C. for 1 h. The reaction mixture was diluted with saturated aqueous sodium bicarbonate (5 mL), extracted with ethyl acetate (3×8 mL), and dried with Na₂SO₄. The mixture was filtered and concentrated. The crude product was purified via semi-preparative HPLC to afford separated title compounds as white solids.

### Example 2

### N-(4-{1-Methyl-4-[2-({3-[2-(4-morpholin-1)ethyl]phenyl}amino)-4-pyrimidinyl]-1H-pyrazol-5-yl}phenyl)benzamide

¹H NMR (400 MHz, DMSO-d₆) δ ppm 10.51 (s, 1H), 9.41 (s, 1H), 8.22 (d, J=5.3 Hz, 1H), 8.11 (s, 1H), 8.03-7.96 (m, 4H), 7.65-7.55 (m, 3H), 7.49-7.44 (m, 3H), 7.13 (t, J=7.8 Hz, 1H), 6.78 (d, J=7.6 Hz, 1H), 6.31 (d, J=5.1 Hz, 2H), 3.71 (s, 3H), 3.61-3.53 (m, 4H), 2.73-2.64 (m, 2H), 2.54-2.50 (m, 2H), 2.45-2.42 (s, 4H); ESI MS (m/z) 560 [M+H]⁺; analytical HPLC t_{R}=4.71 min.

### Example 3

### N-(4-{1-Methyl-4-[2-({3-[2-(4-morpholinyl)ethyl]phenyl}amino)-4-pyrimidinyl]-1H-pyrazol-3-yl}phenyl)benzamide

¹H NMR (400 MHz, DMSO-d₆) δ ppm 10.35 (s, 1H), 9.46-9.40 (m, 1H), 8.32 (d, J=5.1 Hz, 1H), 8.26 (s, 1H), 8.15 (s, 1H), 7.96 (d, J=8.6 Hz, 2H), 7.84 (d, J=8.6 Hz, 2H), 7.61-7.47 (m, 5H), 7.09 (t, J=7.7 Hz, 1H), 6.76 (d, J=7.6 Hz, 1H), 6.61 (d, J=5.1 Hz, 1H), 3.96 (s, 3H), 3.59-3.55 (m, 4H), 2.67-2.63 (m, 2H), 2.54-2.50 (m, 2H), 2.43-2.33 (m, 4H); ESI MS (m/z) 560 [M+H]⁺; analytical HPLC t_{R}=4.56 min.

### Example 4

### N-[4-(4-{2-[(3-Fluorophenyl)amino]-4-pyrimidinyl}-1H-pyrazol-3-yl)phenyl]benzamide

This compound was prepared following a procedure analogous to that outlined for Example 1. ¹H NMR (400 MHz, CD₃OD) δ ppm 8.29 (d, J=5.3 Hz, 1H), 8.29-8.15 (s, 1H), 8.96-8.93 (m, 2H), 7.85 (s, 2H), 7.64-7.53 (m, 6H), 7.30-7.28 (m, 1H), 7.20-7.16 (m, 1H), 6.81-6.79 (m, 1H), 6.67-6.62 (m, 1H); ESI MS (m/z) 451 [M+H]⁺; analytical HPLC t_{R}=5.62 min.

### Example 5

### N-{4-[4-(2-{[3-(4-Methyl-1-piperazinyl)phenyl]amino}-4-pyrimidinyl)-1H-pyrazol-3-yl]phenyl}benzamide

This compound was prepared following a procedure analogous to that outlined for Example 1. ¹H NMR (400 MHz, DMSO-d₆) δ ppm 13.40-13.38 (m, 1H), 10.45-10.36 (m, 1H), 9.31 (s, 1H), 8.29 (d, J=5.0 Hz, 1H), 8.06-7.92 (m, 4H), 7.62-7.51 (m, 5H), 7.42 (s, 1H), 7.15-7.13 (m, 1H), 7.01 (t, J=8.1 Hz, 1H), 6.62 (d, J=5.3 Hz, 1H), 6.50 (dd, J=8.1, 1.8 Hz, 1H), 3.10-3.08 (m, 4H), 2.49-2.41 (m, 4H), 2.22 (s, 3H); ESI MS (m/z) 531 [M+H]⁺; analytical HPLC t_{R}=4.31 min.

### Example 6

### N-(4-{4-[2-({3-[(N,N-Dimethylglycyl)amino]phenyl}amino)-4-pyrimidinyl]-1H-pyrazol-3-yl}phenyl)benzamide

This compound was prepared following a procedure analogous to that outlined for Example 1. ¹H NMR (400 MHz, DMSO-d₆) δ ppm 13.37 (d, J=14.1 Hz, 1H), 10.40 (d, J=32.6 Hz, 1H), 9.56 (s, 1H), 9.49 (s, 1H), 8.47-8.09 (m, 1H), 8.30 (d, J=5.3 Hz, 1H), 8.19-8.14 (m, 1H), 7.97 (d, J=7.3 Hz, 2H), 7.95-7.83 (m, 2H), 7.62-7.50 (m, 5H), 7.35-7.30 (m, 1H), 7.20-7.08 (m, 2H), 6.64-6.62 (m, 1H), 3.07 (s, 2H), 2.29 (s, 6H); ESI MS (m/z) 533 [M+H]⁺; analytical HPLC t_{R}=4.26 min.

### Example 7

### N-{4-[4-(2-{[3-(4-Morpholinylmethyl)phenyl]amino}-4-pyrimidinyl)-1H-pyrazol-3-yl]phenyl}benzamide

This compound was prepared following a procedure analogous to that outlined for Example 1. ¹H NMR (400 MHz, DMSO-d₆) δ ppm 13.37 (d, J=21.5 Hz, 1H), 10.40 (d, J=38.9 Hz, 1H), 9.44-9.51 (m, 1H), 8.32-8.19 (m, 2H), 8.00-7.90 (m, 5H), 7.72-7.48 (m, 5H), 7.13-7.11 (m, 1H), 6.83 (d, J=7.6 Hz, 1H), 6.67-6.64 (m, 1H) 3.60-3.56 (m, 4H), 3.35-3.43 (m, 2H) 2.37-2.34 (s, 4H); ESI MS (m/z) 532 [M+H]⁺; analytical HPLC t_{R}=4.35 min.

### Example 8

### N-{4-[4-(2-{[3-(4-Methyl-1-piperazinyl)phenyl]amino}-4-pyrimidinyl)-1H-pyrazol-3-yl]phenyl}cyclopropanecarboxamide

This compound was prepared following a procedure analogous to that outlined for Example 1. ¹H NMR (400 MHz, DMSO-d₆) δ ppm 13.37-13.35 (m, 1H), 10.41-10.29 (m, 1H), 9.29 (s, 1H), 8.29-8.25 (m, 1.5H), 8.05-8.03 (m, 0.5H), 7.74-7.62 (m, 2H), 7.47-7.38 (m, 3H), 7.14-7.11 (m, 1H), 6.99 (t, J=8.1 Hz, 1H), 6.58 (d, J=5.31 Hz, 1H), 6.50 (dd, J=8.2, 1.9 Hz, 1H), 3.10-3.08 (m, 4H), 2.47-2.40 (m, 4H), 2.22 (s, 3H), 1.85-1.76 (m, 1H), 0.82 (d, J=3.5 Hz, 4H); ESI MS (m/z) 495 [M+H]⁺; analytical HPLC t_{R}=3.82 min.

### Example 9

### N-(4-{4-[2-({3-[2-(4-Morpholinyl)ethyl]phenyl}amino)-4-pyrimidinyl]-1H-pyrazol-3-yl}phenyl)cyclopropanecarboxamide

This compound was prepared following a procedure analogous to the one outlined for Example 1. ¹H NMR (400 MHz, DMSO-d₆) δ ppm 13.34 (s, 1H), 10.40-10.35 (m, 1H), 9.40 (s, 1H), 8.31-8.27 (m, 1.5H), 8.07-8.05 (m, 0.5H), 7.70-7.58 (m, 2H), 7.49-7.42 (m, 3H), 7.06 (t, J=7.8 Hz, 1H), 6.76 (d, J=7.8 Hz, 1H), 6.62 (d, J=5.3 Hz, 1H), 3.61-3.54 (m, 4H), 2.67-2.63 (m, 2H), 2.54-2.50 (m, 2H), 2.42-2.39 (m, 4H), 1.83-1.78 (m, 1H), 0.84-0.79 (m, 4H); ESI MS (m/z) 510 [M+H]⁺; analytical HPLC t_{R}=3.87 min.

### Examples 10 & 11

These compounds were prepared following a procedure analogous to that outlined for Examples 2 and 3.

### Example 10

### N-(4-{1-Methyl-4-[2-({3-[2-(4-morpholinyl)ethyl]phenyl}amino)-4-pyrimidinyl]-1H-pyrazol-yl}phenyl)cyclopropanecarboxamide

¹H NMR (400 MHz, DMSO-d₆) δ ppm 10.46 (s, 1H), 9.40 (s, 1H), 8.20 (d, J=5.3 Hz, 1H), 8.09 (s, 1H), 7.79 (d, J=8.6 Hz, 2H), 7.62 (s, 1H), 7.44-7.39 (m, 3H), 7.11 (t, J=7.8 Hz, 1H), 6.78 (d, J=7.6 Hz, 1H), 6.27 (d, J=5.3 Hz, 1H), 3.68 (s, 3H), 3.62-3.52 (m, 4H), 2.73-2.65 (m, 2H), 2.54-2.50 (m, 2H), 2.44-2.42 (m, 4H), 1.79-1.86 (m, 1H), 0.85-0.83 (m, 4H); ESI MS (m/z) 524 [M+H]⁺; analytical HPLC t_{R}=4.24 min.

### Example 11

### N-(4-{1-Methyl-4-[2-({3-[2-(4-morpholinyl)ethyl]phenyl}amino)-4-pyrimidinyl]-1H-pyrazol-3-yl}phenyl)cyclopropanecarboxamide

¹H NMR (400 MHz, DMSO-d₆) δ ppm 10.28 (s, 1H), 9.41 (s, 1H), 8.30 (d, J=5.1 Hz, 1H), 8.17 (s, 1H), 7.63 (d, J=8.6 Hz, 2H), 7.53 (s, 1H), 7.51-7.44 (s, 1H), 7.46-7.42 (m, 2H), 7.07 (t, J=7.8 Hz, 1H), 6.76 (d, J=7.6 Hz, 1H), 6.58 (d, J=5.1 Hz, 1H), 3.94 (s, 3H), 3.61-3.52 (m, 4H), 2.68-2.60 (m, 2H), 2.54-2.50 (m, 2H), 2.45-2.40 (m, 4H), 1.76-1.83 (m, 1H), 0.77-0.84 (m, 4H); ESI MS (m/z) 524 [M+H]⁺; analytical HPLC t_{R}=4.19 min.

### Examples 12 & 13

These compounds were prepared following a procedure analogous to that outlined for Examples 2 and 3.

### Example 12

### N-{4-[1-Methyl-4-(2-{[3-(4-methylpiperazin-1-yl)phenyl]amino}pyrimidin-4-yl)-1H-pyrazol-5-yl]phenyl}benzamide

¹H NMR (400 MHz, DMSO-d₆) δ ppm 10.51 (s, 1H), 9.29 (s, 1H), 8.20 (d, J=5.3 Hz, 1H), 8.10 (s, 1H), 8.03-7.96 (m, 4H), 7.65-7.55 (m, 3H), 7.51-7.42 (m, 3H), 7.14-7.03 (m, 2H), 6.52 (dd, J=8.1, 1.5 Hz, 1H), 6.28 (d, J=5.3 Hz, 1H), 3.71 (s, 3H), 3.16-3.07 (m, 4H), 2.49-2.42 (m, 4H), 2.24 (s, 3H); ESI MS (m/z) 545 [M+H]⁺; analytical HPLC t_{R}=4.69 min.

### Example 13

### N-{4-[1-Methyl-4-(2-{[3-(4-methylpiperazin-1-yl)phenyl]amino}pyrimidin-4-yl)-1H-pyrazol-3-yl]phenyl}benzamide

¹H NMR (400 MHz, DMSO-d₆) δ ppm 10.36 (s, 1H), 9.31 (s, 1H), 8.30 (d, J=5.3 Hz, 1H), 8.16 (s, 1H), 7.99-7.95 (m, 2H), 7.86-7.82 (m, 2H), 7.61-7.49 (m, 5H), 7.40 (t, J=2.0 Hz, 1H), 7.20-7.14 (m, 1H), 7.03 (t, J=8.1 Hz, 1H), 6.57 (d, J=5.3 Hz, 1H), 6.53-6.46 (m, 1H), 3.96 (s, 3H), 3.12-3.07 (m, 4H), 2.48-2.43 (m, 4H), 2.23 (s, 3H); ESI MS (m/z) 545 [M+H]⁺; analytical HPLC t_{R}=4.52 min.

### Examples 14 & 15

These compounds were prepared following a procedure analogous to that outlined for Examples 2 and 3.

### Example 14

### N-{4-[1-Methyl-4-(2-{[3-(4-methylpiperazin-1-yl)phenyl]amino}pyrimidin-4-yl)-1H-pyrazol-5-yl]phenyl}cyclopropanecarboxamide

¹H NMR (400 MHz, DMSO-d₆) δ ppm 10.45 (s, 1H), 9.28 (s, 1H), 8.20-8.16 (m, 1H), 8.08 (s, 1H), 7.79 (d, J=8.8 Hz, 2H), 7.44-7.38 (m, 3H), 7.13-7.08 (m, 1H), 7.04 (t, J=8.1 Hz, 1H), 6.54-6.50 (m, 1H), 6.24 (d, J=5.1 Hz, 1H), 3.68 (s, 3H), 3.14-3.08 (m, 4H), 2.49-2.41 (m, 4H), 2.23 (s, 3H), 1.88-1.79 (m, 1H), 0.88-0.80 (m, 4H); ESI MS (m/z) 509 [M+H]⁺; analytical HPLC t_{R}=4.07 min.

### Example 15

### N-{4-[1-Methyl-4-(2-{[3-(4-methylpiperazin-1-yl)phenyl]amino}pyrimidin-4-yl)-1H-pyrazol-3-yl]phenyl}cyclopropanecarboxamide

¹H NMR (400 MHz, DMSO-d₆) δ ppm 10.29 (s, 1H), 9.30 (s, 1H), 8.28 (d, J=5.3 Hz, 1H), 8.17 (s, 1H), 7.63 (d, J=8.6 Hz, 2H), 7.45-7.38 (m, 3H), 7.15 (d, J=9.1 Hz, 1H), 7.01 (t, J=8.1 Hz, 1H), 6.54 (d, J=5.0 Hz, 1H), 6.51 (dd, J=8.0, 1.9 Hz, 1H), 3.94 (s, 3H), 3.12-3.04 (m, 4H), 2.48-2.42 (m, 4H), 2.23 (s, 3H), 1.81-1.77 (m, 1H), 0.86-0.77 (m, 4H); ESI MS (m/z) 509 [M+H]⁺; analytical HPLC t_{R}=4.25 min.

### Examples 16 & 17

These compounds were prepared following a procedure analogous to that outlined for Examples 2 and 3.

### Example 16

### N-{4-[1-Ethyl-4-(2-{[3-(4-methyl-1-piperazinyl)phenyl]amino}-4-pyrimidinyl)-1H-pyrazol-5-yl]phenyl}cyclopropanecarboxamide

¹H NMR (400 MHz, DMSO-d₆) δ ppm 10.47 (s, 1H), 9.27 (s, 1H), 8.17 (s, 1H), 8.12 (s, 1H), 7.80 (d, J=8.6 Hz, 2H), 7.46-7.36 (m, 3H), 7.14-7.11 (m, 1H), 7.05 (t, J=8.1 Hz, 1H) 6.52 (dd, J=7.8, 1.8 Hz, 1H), 6.18 (d, J=5.3 Hz, 1H), 3.95 (q, J=7.3 Hz, 2H), 3.14-3.05 (m, 4H), 2.50-2.46 (m, 4H), 2.24 (s, 3H), 1.84-1.82 (m, 1H), 1.26 (t, J=7.2 Hz, 3H), 0.87-0.79 (m, 4H); ESI MS (m/z) 523 [M+H]⁺; analytical HPLC t_{R}=4.53 min.

### Example 17

### N-{4-[1-Ethyl-4-(2-{[3-(4-methylpiperazin-1-yl)phenyl]amino}pyrimidin-4-yl)-1H-pyrazol-3-yl]phenyl}cyclopropanecarboxamide

¹H NMR (400 MHz, DMSO-d₆) δ ppm 10.29 (s, 1H), 9.30 (s, 1H), 8.29-8.26 (m, 1H), 8.15 (s, 1H), 7.63 (d, J=8.6 Hz, 2H), 7.46-7.40 (m, 3H), 7.12 (d, J=9.1 Hz, 1H), 7.00 (t, J=8.2 Hz, 1H), 6.55 (d, J=5.0 Hz, 1H), 6.51 (dd, J=8.3, 1.8 Hz, 1H), 4.03 (q, J=7.1 Hz, 2H), 3.12-3.06 (m, 4H), 2.48-2.39 (m, 4H), 2.23 (s, 3H), 1.83-1.76 (m, 1H), 1.18 (t, J=7.1 Hz, 3H), 0.87-0.77 (m, 4H); ESI MS (m/z) 523 [M+H]⁺; analytical HPLC t_{R}=4.33 min.

### Example 18

### N-(4-{4-[2-({3-[2-(4-Morpholinyl)ethyl]phenyl}amino)-4-pyrimidinyl]-1H-pyrazol-3-yl}phenyl)-1-pyrrolidinecarboxamide

This compound was prepared following a procedure analogous to that outlined for Example 1. ¹H NMR (400 MHz, DMSO-d₆) δ ppm 13.30 (s, 1H), 9.41 (s, 1H), 8.28 (d, J=5.0 Hz, 1H), 8.17 (s, 1H), 7.65-7.60 (m, 3H), 7.50-7.48 (m, 1H), 7.40 (d, J=8.6 Hz, 2H), 7.09 (t, J=7.8 Hz, 1H), 6.77 (d, J=7.6 Hz, 1H), 6.61 (d, J=5.3 Hz, 1H), 3.54-3.62 (m, 4H), 3.41-3.33 (m, 4H), 2.68-2.63 (m, 2H), 2.50-2.46 (m, 2H), 2.44-2.40 (m, 4H), 1.91-1.83 (m, 4H); ESI MS (m/z) 539 [M+H]⁺; analytical HPLC t_{R}=4.03 min.

### Example 19

### N-{4-[4-(2-{[3-(4-Methyl-1-piperazinyl)phenyl]amino}-4-pyrimidinyl)-1H-pyrazol-3-yl]phenyl}-1-pyrrolidinecarboxamide

This compound was prepared following a procedure analogous to that outlined for Example 1. ¹H NMR (400 MHz, DMSO-d₆) δ ppm 13.26 (s, 1H), 9.30 (s, 1H), 8.26 (d, J=5.3 Hz, 1H), 8.18-8.14 (m, 1H), 7.63-7.60 (m, 2H), 7.47-7.42 (m, 1H), 7.39 (d, J=8.6 Hz, 2H), 7.19-7.16 (m, 1H), 7.03 (t, J=8.1 Hz, 1H), 6.57 (d, J=5.0 Hz, 1H), 6.51 (dd, J=8.1, 2.0 Hz, 1H), 3.41-3.36 (m, 4H), 3.14-3.06 (m, 4H), 2.49-2.43 (m, 4H), 2.23 (s, 3H), 1.89-1.83 (m, 4H); ESI MS (m/z) 524 [M+H]⁺; analytical HPLC t_{R}=4.00 min.

### Example 20 & 21

These compounds were prepared following a procedure analogous to that outlined for Examples 2 and 3.

### Example 20

### N-(4-{1-Ethyl-4-[2-({3-[2-(4-morpholinyl)ethyl]phenyl}amino)-4-pyrimidinyl]-1H-pyrazol-5-yl}phenyl)-1-pyrrolidinecarboxamide

¹H NMR (400 MHz, DMSO-d₆) δ ppm 9.40 (s, 1H), 8.39 (s, 1H), 8.17 (d, J=5.3 Hz, 1H), 8.13 (s, 1H), 7.75 (d, J=8.6 Hz, 2H), 7.67 (s, 1H), 7.50 (d, J=8.3 Hz, 1H), 7.31 (d, J=8.8 Hz, 2H), 7.15 (t, J=7.8 Hz, 1H), 6.80 (s, 1H), 6.19 (d, J=5.3 Hz, 1H), 3.96 (q, J=7.1 Hz, 2H), 3.62-3.51 (m, 4H), 3.44-3.38 (m, 4H), 2.73-2.66 (m, 2H), 2.54-2.48 (m, 2H), 2.45-2.40 (m, 4H), 1.91-1.84 (m, 4H), 1.27 (t, J=7.3 Hz, 3H); ESI MS (m/z) 567 [M+H]⁺; analytical HPLC t_{R}=4.60 min.

### Example 21

### N-(4-{1-Ethyl-4-[2-({3-[2-(4-morpholinyl)ethyl]phenyl}amino)-4-pyrimidinyl]-1H-pyrazol-3-yl}Phenyl)-1-pyrrolidinecarboxamide

¹H NMR (400 MHz, DMSO-d₆) δ ppm 9.41 (s, 1H), 8.29 (d, J=5.0 Hz, 1H), 8.21 (s, 1H), 8.17 (s, 1H), 7.61-7.53 (m, 3H), 7.49 (d, J=8.3 Hz, 1H), 7.37 (d, J=8.6 Hz, 2H), 7.10 (t, J=7.8 Hz, 1H), 6.77 (d, J=7.6 Hz, 1H), 6.58 (d, J=5.3 Hz, 1H), 4.23 (q, J=7.2 Hz, 2H), 3.62-3.52 (m, 4H), 3.40-3.33 (m, 4H) 2.70-2.63 (m, 2H), 2.50-2.47 (m, 2H), 2.44-2.40 (m, 4H), 1.91-1.82 (m, 4H), 1.46 (t, J=7.3 Hz, 3H); ESI MS (m/z) 567 [M+H]⁺; analytical HPLC t_{R}=4.45 min.

### Intermediate 7

### Ethyl 4-({[tert-butyloxy]carbonyl}amino)benzoate

This compound was prepared as described by Niimi et al. (Niimi, Tatsuya; Orita, Masaya; Okazawa-Igarashi, Miwa; Sakashita, Hitoshi; Kikuchi, Kazumi; Ball, Evelyn; Ichikawa, Atsushi; Yamagiwa, Yoko; Sakamoto, Shuichi; Tanaka, Akihiro; Tsukamoto, Shinichi; Fujita, Shigeo; Tatsuta, Kuniaki; Maeda, Yasuhide; Chikauchi, Ken., J. Med. Chem. 2001, 44(26), 4737-4740), with the following modification in work-up. The crude mixture was concentrated to dryness and redissolved in ethyl acetate. It was then washed with 1N HCl solution (3×) and dried over MgSO₄. After filtration and full evaporation of the solvent, the crude crystals were washed with hexanes and dried under vacuum to give white crystals. ¹H NMR (400 MHz, DMSO-d₆) δ 9.80 (s, 1H), 7.85 (d, J=8.8 Hz, 2H), 7.58 (d, J=8.8 Hz, 2H), 4.25 (q, J=7.2 Hz, 2H), 1.49 (s, 9H), 1.30 (t, J=7.2 Hz, 3H); ESI MS (m/z) 266 [M+H]⁺; analytical HPLC t_{R}=7.0 min.

### Intermediate 8

### tert-Butyl (4-{[2-(methylthio)-4-pyrimidinyl]acetyl}phenyl)carbamate

The title compound was prepared following the procedure of Intermediate 3, using ethyl 4-({[tert-butyloxy]carbonyl}amino)benzoate (Intermediate 7) as the ester. In the work-up, a cold solution of ammonium chloride was used instead of the hydrochloric acid solution, in order to avoid deprotection of the Boc group. ESI MS (m/z) 360 [M+H]⁺; LC MS retention time t_{R}=2.3 min (ketone) and t_{R}=2.8 min (enol).

### Intermediate 9

### tert-Butyl {4-[4-(2-Methylsulfanyl-pyrimidin-4-yl)-1H-pyrazol-3-yl]-phenyl}carbamate

The title compound was prepared following the procedure of Intermediate 4, using Intermediate 8 as substrate. As modifications of the original procedure, formation of the activated eneamine in dimethylformamide dimethylacetal was achieved at 60° C. for 3 h, followed by 7 hours at room temperature. Purification involved flash column chromatography on silica gel, with a gradient of 10:90 to 30:70 AcOEt/Hexanes. ¹H NMR (400 MHz, DMSO-d₆) δ 13.25 (bs, 1H), 9.54 (s, 1H), 8.41 (d, J=5.2 Hz, 1H), 8.27 (s, 1H), 7.53 (d, J=8.8 Hz, 2H), 7.41 (d, J=8.8 Hz, 2H), 7.03 (d, J=5.2 Hz, 1H), 2.32 (s, 3H), 1.49 (s, 9H); ESI MS (m/z) 384 [M+H]⁺; LCMS retention time t_{R}=2.2 min; analytical HPLC t_{R}=6.1 min.

### Intermediate 10

### tert-Butyl {4-[4-(2-Methanesulfonyl-pyrimidin-4-yl)-1H-pyrazol-3-yl]-phenyl}carbamate

To a solution of intermediate 9 (1.0 g) in 20 mL of a 1:1 THF/MeOH mixture cooled to 0° C. was added dropwise an aqueous solution of Oxone® (6.4 g in 20 mL of water). After 15 min, the reaction was warmed up to room temperature and stirred for an additional hour. Disappearance of starting material and intermediate sulfoxide was followed by HPLC. The mixture was then diluted with 60 mL of a saturated bicarbonate solution, and extracted with ethyl acetate (3×). The organic layers were combined, dried over MgSO₄, and concentrated under vacuum. The compound was purified by flash chromatography on silica gel (gradient of CHCl₃/MeOH/NH₄OH from 100:0:0 to 90:10:1). ESI MS (m/z) 416 [M+H]⁺; LCMS retention time t_{R}=1.8 min; analytical HPLC t_{R} (sulfone)=5.4 min.

### Intermediate 11

### Mixture of tert-butyl (4-{1-ethyl-4-[2-(methylsulfonyl)-4-pyrimidinyl]-1H-pyrazol-3-yl}phenyl) carbamate & tert-butyl (4-{1-ethyl-4-[2-(methylsulfonyl)-4-pyrimidinyl]-1H-pyrazol-5-yl}phenyl carbamate

The title compounds were prepared following the procedure of Example 6, using intermediate 10 as substrate and iodoethane as alkylating agent. ¹H NMR (400 MHz, DMSO-d₆) (mixture (1:1) of 2 regioisomers) δ 9.62 & 9.58 (s, 1H), 8.82 & 8.77 (d, J=4-5 Hz, 1H), 8.66 & 8.36 (s, 1H), 7.66 & 7.53, (d, J=8.1 Hz, 2H), 7.44 & 7.36 (d, J=8.1 Hz, 2H), 4.25 & 3.96 (q, J=8.0 Hz, 2H), 3.25 & 3.08 (s, 3H), 1.51 & 1.50 (s, 9H), 1.42 & 1.26 (t, J=8.0 Hz, 3H); ESI MS (m/z) 444; LCMS retention time t_{R}=2.1 min (broad); analytical HPLC t_{R}=6.12 & 6.27 min.

### Intermediate 12

### Mixture of tert-butyl (4-{1-ethyl-4-[2-({3-[2-(4-morpholinyl)ethyl]phenyl}amino)-4-pyrimidinyl]-1H-pyrazol-3-yl}phenyl) carbamate & tert-butyl (4-{1-ethyl-4-[2-({3-[2-(4-morpholinyl)ethyl]phenyl}amino)-4-pyrimidinyl]-1H-pyrazol-5-yl}phenyl) carbamate

The title compounds were prepared following the procedure outlined for Examples 2 and 3, using Intermediate 11 as substrate. Purification was conducted on the Analogix system with a CHCl₃/MeOH gradient. ESI MS (m/z) 570; LCMS retention time t_{R}=1.9 min (broad); analytical HPLC t_{R}=5.26 & 5.49 min.

### Intermediate 13

### Mixture of 4-[3-(4-aminophenyl)-1-ethyl-1H-pyrazol-4-yl]-N-{3-[2-(4-morpholinyl)ethyl]phenyl}-2-pyrimidinamine & 4-[5-(4-aminophenyl)-1-ethyl-1H-pyrazol-4-yl]-N-{3-[2-(4-morpholinyl)ethyl]phenyl}-2-pyrimidinamine

Boc deprotection of Intermediate 12 was successfully achieved by treatment with 20% trifluoroacetic acid in methylene chloride for 30 min. The reaction mixture was then concentrated to dryness and azeotroped with toluene. ESI MS (m/z) 470 [M+H]⁺; analytical HPLC t_{R}=3.50 & 3.80 min.

### Examples 22 and 23

Intermediate 13 was dissolved in pyridine at 0° C., followed by addition of one equivalent of ethyl isocyanate. The reaction was then warmed up to room temperature and stirred for several hours. Disappearance of starting material was followed by HPLC. The reaction mixture was then diluted with water and ethyl acetate, washed with 1M HCl (3×) and a saturated solution of sodium bicarbonate. The organic layers were combined, concentrated under vacuum (60° C.). The residue was azeotroped three times with toluene. Crude compounds were there purified by preparative HPLC.

### Example 22

### N-ethyl-N′-(4-{1-ethyl-4-[2-({3-[2-(4-morpholinyl)ethyl]phenyl}amino)-4-pyrimidinyl]-1H-pyrazol-5-yl}phenyl)urea

¹H NMR (400 MHz, CD₃OD) δ8.22 (s, 1H), 8.15 (bs, 1H), 8.10 (d, J=4.1 Hz, 1H), 7.69 (s, 1H), 7.62 (d, J=8.1 Hz, 2H), 7.48 (d, J=8.0 Hz, 1H), 7.31 (d, J=8.1 Hz, 2H), 7.27 (t, J=8.0 Hz, 1H), 6.94 (d, J=7.9 Hz, 1H), 6.33 (d, J=8.0 Hz, 1H), 4.06 (q, J=4.0 Hz, 2H), 3.94 (bs, 2H), 3.42-3.35 (m, 6H), 3.28 (q, J=4.0 Hz, 2H), 3.10-3.06 (m, 2H), 1.35 (t, J=4.0 Hz, 3H), 1.20 (t, J=4.0 Hz, 3H); ESI MS (m/z) 541 [M+H]⁺; LCMS retention time t_{R}=1.47 min; analytical HPLC t_{R}=4.35 min.

### Example 23

### N-ethyl-N′-(4-{1-ethyl-4-[2-({3-[2-(4-morpholinyl)ethyl]phenyl}amino)-4-pyrimidinyl]-1H-pyrazol-3-yl}phenyl)urea

¹H NMR (400 MHz, CD₃OD) δ8.25 (s, 1H); 8.24 (d, J=4.1 Hz, 1H), 8.09 (bs, 1H), 7.54 (s, 1H), 7.48 (d, J=8.1 Hz, 2H), 7.41 (m, 3H), 7.24 (t, J=8.0 Hz, 1H), 6.91 (d, J=7.9 Hz, 1H), 6.75 (d, J=8.0 Hz, 1H), 4.28 (q, J=4.0 Hz, 2H), 3.94 (bs, 2H), 3.40-3.30 (m, 6H), 3.14 (q, J=4.0 Hz, 2H), 3.01-2.96 (m, 2H), 1.56 (t, J=4.0 Hz, 3H), 1.19 (t, J=4.0 Hz, 3H); ESI MS (m/z) 541 [M+H]⁺; LCMS retention time t_{R}=1.48 min; analytical HPLC t_{R}=4.16 min.

### Examples 24 and 25

These compounds were prepared following a procedure analogous to the one outlined for Examples 22 and 23, using Intermediate 13 and n-propyl isocyanate as reagents.

### Example 24

### N-propyl-N′-(4-{1-ethyl-4-[2-({3-[2-(4-morpholinyl)ethyl]phenyl}amino)-4-pyrimidinyl]-1H-pyrazol-5-yl}phenyl)urea

ESI MS (m/z) 555 [M+H]⁺; LCMS retention time t_{R}=1.52 min; analytical HPLC t_{R}=4.54 min.

### Example 25

### N-propyl-N′-(4-{1-ethyl-4-[2-({3-[2-(4-morpholinyl)ethyl]phenyl}amino)-4-pyrimidinyl]-1H-pyrazol-3-yl}phenyl)urea

¹H NMR (400 MHz, DMSO-d₆) δ 9.42 (s, 1H), 8.53 (s, 1H), 8.29 (d, J=4.0 Hz, 1H), 8.26 (s, 1H), 8.14 (bs, 2H), 7.59 (s, 1H), 7.47 (d, J=12.1 Hz, 1H), 7.42 (d, J=8.1 Hz, 2H), 7.36 (d, J=8.1 Hz, 2H), 7.09 (t, J=8.0 Hz, 1H), 6.77 (d, J=8.0 Hz, 1H), 6.58 (d, J=4.0 Hz, 1H), 6.18 (t, J=4.0 Hz, 1H), 5.75 (m, 2H), 4.22 (q, J=4.0 Hz, 2H), 3.60 (m, 4H), 3.05 (q, J=4.1 Hz, 2H), 2.92 (q, J=4.0 Hz, 4H), 2.70-2.65 (m, 2H), 1.49-1.43 (m, 5H), 0.88 (t, J=4.0 Hz, 3H); ESI MS (m/z) 555 [M+H]⁺; LCMS retention time t_{R}=1.50 min; analytical HPLC t_{R}=4.39 min.

### Examples 26 and 27

To a solution of 50 mg of Intermediate 13 in THF was added dropwise a 20% phosgene solution in toluene (1 equivalent). The reaction mixture was stirred at 0° C. for 30 min, before addition of cyclopropylamine (2 equivalents). The reaction was warmed up to room temperature and stirred for an additional hour. Disappearance of starting material was followed by HPLC. The reaction was then diluted with water and ethyl acetate. After decantation, aqueous layer was extracted three times with AcOEt. The organic layers were combined, dried over MgSO₄ and concentrated under vacuum. Crude compounds were there purified by preparative HPLC.

### Example 26

### N-cyclopropyl-N′-(4-{1-ethyl-4-[2-({3-[2-(4-morpholinyl)ethyl]phenyl}amino)-4-pyrimidinyl]-1H-pyrazol-5-yl}phenyl)urea

¹H NMR (400 MHz, DMSO-d₆) δ 9.53 (s, 1H), 8.63 (s, 1H), 8.19 (d, J=4.0 Hz, 1H), 8.15 (s, 1H), 7.73 (s, 1H), 7.62 (d, J=8.1 Hz, 2H), 7.55 (d, J=12.1 Hz, 1H), 7.30 (d, J=8.1 Hz, 2H), 7.23 (t, J=8.0 Hz, 1H), 6.84 (d, J=8.0 Hz, 1H), 6.54 (m, 1H), 6.22 (d, J=4.0 Hz, 1H), 4.03 (d, J=12.0 Hz, 2H), 3.95 (q, J=8.0 Hz, 2H), 3.54 (m, 2H); 3.15 (m, 1H), 2.97 (m, 1H), 1.27 (t, J=8.0 Hz, 3H), 1.27 (m, 1H), 0.67 (m, 2H), 0.43 (m, 2H); ESI MS (m/z) 553 [M+H]⁺; LCMS retention time t_{R}=1.52 min; analytical HPLC t_{R}=4.35 min.

### Example 27

### N-cyclopropyl-N′-(4-{1-ethyl-4-[2-({3-[2-(4-morpholinyl)ethyl]phenyl}amino)-4-pyrimidinyl]-1H-pyrazol-3-yl}phenyl)urea

¹H NMR (400 MHz, DMSO-d₆) δ 9.53 (s, 1H), 8.42 (s, 1H), 8.31 (d, J=4.0 Hz, 1H), 8.29 (s, 1H), 7.62 (s, 1H), 7.53 (d, J=12.1 Hz, 1H), 7.44 (d, J=8.1 Hz, 2H), 7.38 (d, J=8.1 Hz, 2H), 7.17 (t, J=8.0 Hz, 1H), 6.81 (d, J=8.0 Hz, 1H), 6.63 (d, J=4.0 Hz, 1H), 6.44 (m, 1H), 4.23 (q, J=8.0 Hz, 2H), 4.02 (d, J=12.0 Hz, 2H), 3.66 (t, J=12.0 Hz, 2H), 3.52 (d, J=12.0 Hz, 2H), 2.89 (m, 1H), 2.67 (m, 1H), 2.55 (m, 1H), 2.33 (m, 1H), 1.46 (t, J=8.0 Hz, 3H), 1.24 (m, 1H), 0.64 (m, 2H), 0.41 (m, 2H); ESI MS (m/z) 553 [M+H]⁺; LCMS retention time tR=1.48 min; analytical HPLC tR=4.23 min.

Examples 28-53 were prepared following procedures analogous to that outlined for Examples 26 & 27.

### Example 28

### N-(4-{1-(1-methylethyl)-4-[2-({3-[2-(4-morpholinyl)ethyl]phenyl}amino)-4-pyrimidinyl]-1H-pyrazol-3-yl}phenyl)cyclopropanecarboxamide

¹H NMR (400 MHz, DMSO-d₆) δ ppm 10.28 (s, 1H), 9.42 (s, 1H), 8.32-8.28 (m, 2H), 7.63 (d, J=8.59 Hz, 2H), 7.59 (s, 1H), 7.44 (d, J=8.84 Hz, 2H), 7.07-7.05 (m, 2H), 6.77 (s, 1H), 6.62 (d, J=5.05 Hz, 1H), 4.64-4.55 (m, 1H), 4.12-4.10 (m, 2H), 3.67-3.65 (m, 4H), 3.35-3.33 (m, 4H), 3.17 (d, J=5.31 Hz, 2H), 1.80-1.78 (m, 1H), 1.50 (d, J=6.82 Hz, 6H), 0.81-0.79 (m, 4H); ESI MS (m/z) 552 [M+H]⁺; LCMS retention time t_{R}=1.58 min: analytical HPLC t_{R}=4.63 min.

### Example 29

### N-(4-{1-(1-methylethyl)-4-[2-({3-[2-(4-morpholinyl)ethyl]phenyl}amino)-4-pyrimidinyl]-1H-pyrazol-5-yl}phenyl)cyclopropanecarboxamide

¹H NMR (400 MHz, DMSO-d₆) δ ppm 10.48 (s, 1H), 9.52 (s, 1H), 8.20-8.16 (m, 2H), 7.81 (d, J=8.59 Hz, 2H), 7.73 (s, 1H), 7.52-7.51 (m, 1H), 7.37 (d, J=8.59 Hz, 2H), 7.23-7.22 (m, 1H), 6.86-6.85 (m, 1H), 6.17 (d, J=5.31 Hz, 1H), 4.26-4.24 (m, 1H), 4.03-4.00 (m, 2H), 3.65-3.48 (m, 4H), 3.15-2.95 (m, 4H), 2.45-2.43 (m, 2H), 1.84-1.83 (m, 1H), 1.36 (d, J=6.57 Hz, 6H), 0.84 (d, J=6.06 Hz, 4H); ESI MS (m/z) 552: LCMS retention time t_{R}=1.60 min: analytical HPLC t_{R}=4.77 min.

### Example 30

### N-(4-{1-ethyl-4-[2-({3-[2-(4-morpholinyl)ethyl]phenyl}amino)-4-pyrimidinyl]-1H-pyrazol-3-yl}phenyl)cyclopropanecarboxamide

¹H NMR (400 MHz, DMSO-d₆) δ ppm 10.29 (s, 1H), 8.32-8.30 (m, 2H), 8.14 (s, 1H), 7.62 (d, J=8.84 Hz, 2H), 7.58 (s, 1H), 7.44 (d, J=8.59 Hz, 2H), 7.10-7.09 (m, 1H), 6.79-6.78 (m, 1H), 6.62-6.60 (m, 1H), 4.11 (t, J=5.05 Hz, 2H), 3.60-3.50 (m, 4H), 3.39-3.30 (m, 4H), 3.20-3.18 (m, 2H), 2.50-2.46 (m, 2H), 1.81-1.78 (s, 1H), 1.46 (t, J=7.33 Hz, 3H), 0.82-0.79 (m, 4H); ESI MS (m/z) 538: LCMS retention time t_{R}=1.50 min: analytical HPLC t_{R}=4.35 min.

### Example 31

### N-(4-{1-ethyl-4-[2-({3-[2-(4-morpholinyl)ethyl]phenyl}amino)-4-pyrimidinyl]-1H-pyrazol-5-yl}phenyl)cyclopropanecarboxamide

¹H NMR (400 MHz, DMSO-d₆) δ ppm 10.47 (s, 1H), 9.52 (s, 1H), 8.20-8.15 (m, 2H), 7.81 (d, J=8.84 Hz, 1H), 7.70 (s, 1H), 7.52-7.51 (m, 1H), 7.38 (d, J=8.59 Hz, 2H), 7.24-7.22 (m, 1H), 6.84-6.83 (m, 1H), 6.23-6.22 (m, 1H), 4.00-3.92 (m, 2H), 3.66-3.44 (m, 6H), 3.17-2.90 (m, 4H), 2.51-2.33 (m, 2H), 1.84-1.82 (m, 1H), 1.27 (t, J=7.20 Hz, 3H), 0.85-0.84 (m, 4H); ESI MS (m/z) 538: LCMS retention time t_{R}=1.60 min: analytical HPLC t_{R}=4.53 min.

### Example 32

### N-(4-{1-(2-hydroxyethyl)-4-[2-({3-[2-(4-morpholinyl)ethyl]phenyl}amino)-4-pyrimidinyl]-1H-pyrazol-3-yl}phenyl)cyclopropanecarboxamide

¹H NMR (400 MHz, DMSO-d₆) δ ppm 10.29 (s, 1H), 9.45 (s, 1H), 8.30 (d, J=5.05 Hz, 1H), 8.26 (s, 1H), 7.63 (d, J=8.84 Hz, 2H), 7.60-7.58 (m, 1H), 7.50-7.48 (s, 1H), 7.44 (d, J=8.84 Hz, 2H), 7.08-7.07 (m, 1H), 6.79-6.78 (m, 1H), 6.59 (d, J=5.05 Hz, 1H), 4.24 (t, J=5.43 Hz, 2H), 3.81 (t, J=5.18 Hz, 2H), 3.62-3.58 (m, 4H), 3.39-3.32 (m, 2H), 2.68-2.67 (m, 2H), 2.51-2.33 (m, 4H), 1.79-1.76 (m, 1H), 0.85-0.76 (m, 4H); ESI MS (m/z) 554: LCMS retention time t_{R}=1.37 min: analytical HPLC t_{R}=3.85 min.

### Example 33

### N-(4-{1-[2-(methyloxy)ethyl]-4-[2-({3-[2-(4-morpholinyl)ethyl]phenyl}amino)-4-pyrimidinyl]-1H-pyrazol-3-yl}phenyl)cyclopropanecarboxamide

¹H NMR (400 MHz, DMSO-d₆) δ ppm 10.29 (s, 1H), 9.43 (s, 1H), 8.30 (d, J=5.31 Hz, 1H), 8.25 (s, 1H), 7.63 (d, J=8.59 Hz, 2H), 7.55 (s, 1H), 7.47 (d, J=10.61 Hz, 1H), 7.46-7.42 (m, 2H), 7.07 (t, J=7.83 Hz, 1H), 6.77 (d, J=7.58 Hz, 1H), 6.59 (d, J=5.31 Hz, 1H), 4.36 (t, J=5.18 Hz, 2H), 3.76 (t, J=5.18 Hz, 2H), 3.62-3.56 (m, 4H), 3.43-3.30 (m, 2H), 3.28 (s, 3H), 3.19-3.16 (m, 4H), 2.69-2.63 (m, 2H), 1.84-1.75 (m, 1H), 0.86-0.77 (m, 4H); ESI MS (m/z) 568: LCMS retention time t_{R}=1.53 min: analytical HPLC t_{R}=4.08 min.

### Example 34

### N-(4-{1-[2-(methyloxy)ethyl]-4-[2-({3-[2-(4-morpholinyl)ethyl]phenyl}amino)-4-pyrimidinyl]-1H-pyrazol-5-yl}phenyl)cyclopropanecarboxamide

¹H NMR (400 MHz, DMSO-d₆) δ ppm 10.46 (s, 1H), 9.42 (s, 1H), 8.19 (d, J=5.31 Hz, 1H), 8.16-8.13 (m, 1H), 7.79 (d, J=8.59 Hz, 2H), 7.64 (s, 1H), 7.46 (d, J=7.83 Hz, 1H), 7.37 (d, J=8.84 Hz, 2H), 7.13 (t, J=7.96 Hz, 1H), 6.79 (d, J=7.58 Hz, 1H), 6.21 (d, J=5.31 Hz, 1H), 4.09-4.06 (m, 2H), 3.69-3.58 (m, 2H), 3.34-3.25 (m, 8H), 3.17 (s, 3H), 2.72-2.54 (m, 4H), 0.86-0.84 (m, 1H), 0.84-0.82 (m, 4H); ESI MS (m/z) 568: LCMS retention time t_{R}=1.57 min: analytical HPLC t_{R}=4.37 min.

### Example 35

### N-(4-{1-(2-methylpropyl)-4-[2-({3-[2-(4-morpholinyl)ethyl]phenyl}amino)-4-pyrimidinyl]-1H-pyrazol-3-yl}phenyl)cyclopropanecarboxamide

¹H NMR (400 MHz, DMSO-d₆) δ ppm 10.29 (s, 1H), 9.42 (s, 1H), 8.30 (d, J=5.31 Hz, 1H), 8.25 (s, 1H), 7.63 (d, J=8.84 Hz, 2H), 7.58 (s, 1H), 7.45-7.43 (m, 3H), 7.06 (t, J=7.83 Hz, 1H), 6.76 (d, J=7.58 Hz, 1H), 6.60 (d, J=5.05 Hz, 1H), 4.11 (d, J=5.05 Hz, 2H), 4.01 (d, J=7.07 Hz, 2H), 3.62-3.54 (m, 4H), 3.17-3.15 (m, 4H), 2.69-2.44 (m, 2H), 2.25-2.14 (m, 1H), 1.83-1.75 (m, 1H), 0.91 (d, J=6.82 Hz, 6H), 0.86-0.76 (m, 4H); ESI MS (m/z) 566: LCMS retention time t_{R}=1.71 min: analytical HPLC t_{R}=4.28 min.

### Example 36

### N-(4-{1-(2-methylpropyl)-4-[2-({3-[2-(4-morpholinyl)ethyl]phenyl}amino)-4-pyrimidinyl]-1H-pyrazol-5-yl}phenyl)cyclopropanecarboxamide

¹H NMR (400 MHz, DMSO-d₆) δ ppm 10.47 (s, 1H), 8.19-8.12 (m, 2H), 7.80 (d, J=8.59 Hz, 2H), 7.68 (s, 1H), 7.52-7.50 (m, 1H), 7.35 (d, J=8.59 Hz, 2H), 7.21-7.19 (m, 1H), 6.84-6.82 (m, 1H), 6.19 (d, J=5.31 Hz, 1H), 4.10-4.01 (m, 2H), 4.78-4.76 (m, 4H), 3.66-3.60 (m, 4H), 3.30-3.17 (m, 4H), 2.10-1.99 (m, 1H), 1.86-1.78 (m, 1H), 0.84 (d, J=6.06 Hz, 4H), 0.73 (d, J=6.82 Hz, 6H); ESI MS (m/z) 566: LCMS retention time t_{R}=1.72 min: analytical HPLC t_{R}=5.11 min.

### Example 37

### N-(4-{1-(methylsulfonyl)-4-[2-({3-[2-(4-morpholinyl)ethyl]phenyl}amino)-4-pyrimidinyl]-1H-pyrazol-3-yl}phenyl)cyclopropanecarboxamide

¹H NMR (400 MHz, DMSO-d₆) δ ppm 10.36 (s, 1H), 9.54 (s, 1H), 8.72 (s, 1H), 8.45 (d, J=5.05 Hz, 1H), 7.68 (d, J=8.84 Hz, 2H), 7.52 (d, J=8.59 Hz, 2H), 7.47 (s, 1H), 7.30 (s, 1H), 7.00 (t, J=7.96 Hz, 1H), 6.81 (d, J=5.05 Hz, 1H), 6.76 (d, J=7.58 Hz, 1H), 3.70 (s, 3H), 3.61-3.55 (m, 4H), 3.18-3.16 (s, 2H), 2.64-2.28 (m, 6H), 1.82-1.76 (m, 1H), 0.85-0.77 (m, 4H); ESI MS (m/z) 588: LCMS retention time t_{R}=1.73 min: analytical HPLC t_{R}=4.76 min.

### Example 38

### N-(4-{1-(2-hydroxyethyl)-4-[2-({3-[2-(4-morpholinyl)ethyl]phenyl}amino)-4-pyrimidinyl]-1H-pyrazol-5-yl}phenyl)cyclopropanecarboxamide

¹H NMR (400 MHz, DMSO-d₆) δ ppm 10.47 (s, 1H), 9.51 (s, 1H), 8.25-8.13 (m, 2H), 7.79 (d, J=8.84 Hz, 2H), 7.69 (s, 1H), 7.51-7.50 (m, 1H), 7.40 (d, J=8.59 Hz, 2H), 7.21-7.19 (m, 1H), 6.85-6.84 (s, 1H), 6.22 (d, J=5.31 Hz, 1H), 4.12-3.96 (m, 2H), 3.74-3.53 (m, 6H), 3.17-2.97 (m, 6H), 2.57-2.53 (m, 2H), 1.86-1.79 (m, 1H), 0.84 (d, J=6.06 Hz, 4H); ESI MS (m/z) 554: LCMS retention time t_{R}=1.43 min: analytical HPLC t_{R}=3.89 min.

### Example 39

### N-(4-{4-[2-({3-[2-(4-morpholinyl)ethyl]phenyl}amino)-4-pyrimidinyl]-1-[2-oxo-2-(1-pyrrolidinyl)ethyl]-1H-pyrazol-3-yl}phenyl)cyclopropanecarboxamide

¹H NMR (400 MHz, DMSO-d₆) δ ppm 10.29 (s, 1H), 9.43 (s, 1H), 8.35-8.28 (m, 2H), 7.63 (d, J=8.59 Hz, 2H), 7.54 (s, 1H), 7.44 (d, J=8.59 Hz, 2H), 7.08-7.06 (m, 1H), 6.78-6.77 (m, 1H), 6.59 (d, J=5.30 Hz, 1H), 5.15 (s, 2H), 3.60-3.17 (m, 10H), 2.38-2.33 (m, 6H), 1.95-1.93 (m, 1H), 1.85-1.74 (m, 4H), 0.85-0.78 (m, 4H); ESI MS (m/z) 621: LCMS retention time t_{R}=1.56 min: analytical HPLC t_{R}=4.24 min.

### Example 40

### N-{4-[4-[2-({3-[2-(4-morpholinyl)ethyl]phenyl}amino)-4-pyrimidinyl]-1-(2,2,2-trifluoroethyl)-1H-pyrazol-3-yl]phenyl}cyclopropanecarboxamide

¹H NMR (400 MHz, DMSO-d₆) δ ppm 10.32 (s, 1H), 9.49 (s, 1H), 8.39 (s, 1H), 8.34 (d, J=5.31 Hz, 1H), 7.65 (d, J=8.59 Hz, 2H), 7.53 (s, 1H), 7.48-7.43 (m, 3H), 7.07 (t, J=7.83 Hz, 1H), 6.77 (d, J=7.58 Hz, 1H), 6.60 (d, J=5.31 Hz, 1H), 5.29 (q, J=9.09 Hz, 2H), 3.58-3.53 (m, 4H), 2.68-2.60 (m, 2H), 2.49-2.40 (m, 6H), 1.83-1.75 (m, 1H), 0.84-0.76 (m, 4H); ESI MS (m/z) 592: LCMS retention time t_{R}=1.65 min: analytical HPLC t_{R}=4.85 min.

### Example 41

### 3-{4-[(cyclopropylcarbonyl)amino]phenyl}-N-ethyl-4-[2-({3-[2-(4-morpholinyl)ethyl]phenyl}amino)-4-pyrimidinyl]-1H-pyrazole-1-carboxamide

¹H NMR (400 MHz, DMSO-d₆) δ ppm 10.35 (s, 1H), 9.51 (s, 1H), 8.75 (s, 1H), 8.70 (t, J=6.06 Hz, 1H), 8.39 (d, J=5.05 Hz, 1H), 7.68 (d, J=8.59 Hz, 2H), 7.56-7.54 (m, 3H), 7.36 (d, J=7.07 Hz, 1H), 7.04 (t, J=7.83 Hz, 1H), 6.76 (d, J=7.58 Hz, 1H), 6.73 (d, J=5.31 Hz, 1H), 3.60-3.52 (m, 4H), 3.39-3.28 (m, 2H), 2.68-2.58 (m, 2H), 2.50-2.33 (m, 6H), 1.79 (t, J=6.06 Hz, 1H), 1.16 (t, J=7.07 Hz, 3H), 0.85-0.77 (m, 4H); ESI MS (m/z) 581: LCMS retention time t_{R}=1.71 min: analytical HPLC t_{R}=4.97 min.

### Example 42

### N-(4-{1-(3-hydroxypropyl)-4-[2-({3-[2-(4-morpholinyl)ethyl]phenyl}amino)-4-pyrimidinyl]-1H-pyrazol-3-yl}phenyl)cyclopropanecarboxamide

¹H NMR (400 MHz, DMSO-d₆) δ ppm 10.29 (s, 1H), 9.42 (s, 1H), 8.30 (d, J=5.05 Hz, 1H), 8.26 (s, 1H), 7.63 (d, J=8.84 Hz, 2H), 7.56 (s, 1H), 7.47-7.42 (m, 3H), 7.06 (t, J=7.83 Hz, 2H), 6.76 (d, J=7.58 Hz, 1H), 6.60 (d, J=5.31 Hz, 1H), 4.26 (t, J=6.95 Hz, 2H), 3.63-3.53 (m, 4H), 3.48-3.40 (m, 2H), 3.17-3.16 (m, 2H), 2.69-2.60 (m, 2H), 2.50-2.44 (m, 4H), 2.04-1.95 (m, 2H), 1.82-1.75 (m, 1H), 0.84-0.76 (m, 4H); ESI MS (m/z) 568: LCMS retention time t_{R}=1.41 min: analytical HPLC t_{R}=3.95 min.

### Example 43

### N-(4-{1-(3-hydroxypropyl)-4-[2-({3-[2-(4-morpholinyl)ethyl]phenyl}amino)-4-pyrimidinyl]-1H-pyrazol-5-yl}phenyl)cyclopropanecarboxamide

¹H NMR (400 MHz, DMSO-_{d6}) δ ppm 10.48 (s, 1H), 9.40 (s, 1H), 8.32-8.25 (m, 1H), 8.18 (d, J=5.05 Hz, 1H), 7.79 (d, J=8.59 Hz, 2H), 7.64 (s, 1H), 7.46-7.45 (m, 1H), 7.37 (d, J=8.59 Hz, 2H), 7.12 (t, J=7.83 Hz, 1H), 6.78 (d, J=7.58 Hz, 1H), 6.19 (d, J=5.31 Hz, 1H), 4.03-3.92 (m, 2H), 3.62-3.53 (m, 4H), 3.34 (t, J=6.06 Hz, 2H), 3.18-3.17 (m, 2H), 2.74-2.63 (m, 2H), 2.50-2.43 (s, 4H), 1.89-1.79 (m, 3H), 0.85-0.83 (m, 4H); ESI MS (m/z) 568: LCMS retention time t_{R}=1.41 min: analytical HPLC t_{R}=3.99 min.

### Example 44

### N-(4-{1-[(2S)-2,3-dihydroxypropyl]-4-[2-({3-[2-(4-morpholinyl)ethyl]phenyl}amino)-4-pyrimidinyl]-1H-pyrazol-3-yl}phenyl)cyclopropanecarboxamide

¹H NMR (400 MHz, DMSO-d₆) δ ppm 10.31 (s, 1H), 9.43 (s, 1H), 8.30-8.28 (m, 1H), 8.23 (s, 1H), 7.65-7.62 (m, 2H), 7.57 (s, 1H), 7.48-7.43 (m, 3H), 7.08-7.04 (m, 1H), 6.77-6.75 (m, 1H), 6.58-6.57 (m, 1H), 4.30-4.28 (m, 1H), 4.07-4.03 (m, 1H), 3.89-3.87 (m, 1H), 3.60-3.18 (m, 8H), 2.67-2.63 (m, 2H), 2.50-2.33 (m, 4H), 1.80-1.79 (m, 1H), 0.81-0.79 (m, 4H); ESI MS (m/z) 584: LCMS retention time t_{R}=1.31 min: analytical HPLC t_{R}=3.73 min.

### Example 45

### N-(4-{1-[(2R)-2,3-dihydroxypropyl]-4-[2-({3-[2-(4-morpholinyl)ethyl]phenyl}amino-4-pyrimidinyl]-1H-pyrazol-3-yl}phenyl)cyclopropanecarboxamide

¹H NMR (400 MHz, DMSO-d₆) δ ppm 10.30 (s, 1H), 9.44 (s, 1H), 8.30-2.28 (m, 2H), 7.64 (d, J=8.59 Hz, 2H), 7.57 (s, 1H), 7.49-7.42 (m, 3H), 7.07 (t, J=7.71 Hz, 1H), 6.76 (d, J=7.58 Hz, 1H), 6.58 (d, J=5.05 Hz, 1H), 4.32 (dd, J=13.89, 3.54 Hz, 1H), 4.06 (dd, J=13.89, 8.08 Hz, 1H), 3.87-3.76 (m, 1H), 3.66-3.17 (m, 6H), 2.68-2.62 (m, 2H), 2.51-2.46 (m, 6H), 1.84-1.74 (m, 1H), 0.84-0.75 (m, 4H); ESI MS (m/z) 584: LCMS retention time t_{R}=1.27 min: analytical HPLC t_{R}=3.72 min.

### Example 46

### N-(4-{1-(3-hydroxy propyl)-4-[2-({3-[2-(4-morpholinyl)ethyl]phenyl}amino)-4-pyrimidinyl]-1H-pyrazol-3-yl}phenyl)-1-pyrrolidinecarboxamide

¹H NMR (400 MHz, DMSO-d₆) δ ppm 9.41 (m, 1H), 8.29 (d, J=5.31 Hz, 1H), 8.26-8.17 (m, 3H), 7.63-7.54 (m, 3H), 7.50-7.48 (m, 1H), 7.37 (d, J=8.59 Hz, 2H), 7.09 (t, J=7.96 Hz, 1H), 6.77 (d, J=7.58 Hz, 1H), 6.58 (d, J=5.05 Hz, 1H), 4.25 (t, J=7.07 Hz, 2H), 3.66-3.17 (m, 12H), 3.59-3.55 (m, 2H), 2.59-2.40 (m, 4H), 2.05-1.95 (m, 2H), 1.82-1.91 (m, 4H); ESI MS (m/z) 597: LCMS retention time t_{R}=1.47 min: analytical HPLC t_{R}=3.77 min.

### Example 47

### N-(4-{1-[(2R)-2,3-dihydroxypropyl]-4-[2-({3-[2-(4-morpholinyl)ethyl]phenyl}amino)-4-pyrimidinyl]-1H-pyrazol-3-yl}phenyl)-1-pyrrolidinecarboxamide

¹H NMR (400 MHz, DMSO-d₆) δ ppm 9.44 (s, 1H), 8.29-8.20 (m, 2H), 7.60-7.56 (m, 3H), 7.50-7.48 (m, 1H), 7.40-7.33 (m, 2H), 7.10 (t, J=7.83 Hz, 1H), 6.79-6.77 (m, 1H), 6.56 (d, J=5.31 Hz, 1H), 4.33-4.28 (m, 1H), 4.06-4.05 (m, 1H), 3.99-3.89 (m, 1H), 3.66-3.16 (m, 12H), 2.70-2.62 (m, 2H), 2.50-2.33 (m, 4H), 1.91-1.84 (m, 4H); ESI MS (m/z) 613: LCMS retention time t_{R}=2.07 min: analytical HPLC t_{R}=3.76 min.

### Example 48

### N-(4-{1-[(2S)-2,3-dihydroxypropyl]-4-[2-({3-[2-(4-morpholinyl)ethyl]phenyl}amino-4-pyrimidinyl]-1H-pyrazol-3-yl}phenyl)-1-pyrrolidinecarboxamide

¹H NMR (400 MHz, DMSO-d₆) δ ppm 9.44 (s, 1H), 8.29-8.22 (m, 3H), 7.61-7.57 (m, 3H), 7.51-7.49 (m, 1H), 7.37 (d, J=8.84 Hz, 2H), 7.12-7.08 (m, 1H), 6.79-6.77 (m, 1H), 6.57-6.58 (m, 1H), 4.35-4.30 (m, 1H), 4.10-4.04 (m, 1H), 3.90-3.85 (m, 1H), 3.61-3.29 (m, 12H), 2.69-2.65 (m, 2H), 2.51-2.33 (m, 4H), 1.91-1.83 (m, 4H); ESI MS (m/z) 613: LCMS retention time t_{R}=1.32 min: analytical HPLC t_{R}=3.78 min.

### Example 49

### N,N-diethyl-N′-{4-[1-(2-hydroxyethyl)-4-(2-{[3-(4-methyl-1-piperazinyl phenyl]amino}-4-pyrimidinyl)-1H-pyrazol-3-yl]phenyl}urea

¹H NMR (400 MHz, DMSO-d₆) δ ppm 10.35 (s, 1H), 9.52 (s, 1H), 8.33-8.26 (m, 3H), 7.56 (d, J=8.84 Hz, 2H), 7.50 (s, 1H), 7.38 (d, J=8.59 Hz, 2H), 7.20-7.18 (m, 1H), 7.11 (t, J=8.08 Hz, 1H), 6.60-6.58 (m, 2H), 4.25 (t, J=5.18 Hz, 2H), 3.82 (t, J=5.31 Hz, 2H), 3.72-3.70 (m, 2H), 3.51-3.49 (m, 2H), 3.36 (q, J=7.07 Hz, 4H), 3.16 (d, J=11.62 Hz, 2H), 3.03-3.00 (m, 2H), 2.83 (d, J=4.80 Hz, 3H), 1.10 (t, J=7.07 Hz, 6H); ESI MS (m/z) 570: LCMS retention time t_{R}=1.38 min: analytical HPLC t_{R}=2.04 min.

### Example 50

### N′-{4-[1-(2-hydroxyethyl)-4-(2-{[3-(1-pyrrolidinylmethyl)phenyl]amino}-4-pyrimidinyl)-1H-pyrazol-3-yl]phenyl}-N,N-dimethylurea

¹H NMR (400 MHz, DMSO-d₆) δ ppm 10.33 (s, 1H), 9.75 (s, 1H), 8.42 (s, 1H), 8.38-8.31 (m, 1H) 7.92 (s, 1H), 7.61-7.59 (m, 1H), 7.53 (d, J=8.59 Hz, 2H), 7.38 (d, J=8.59 Hz, 2H), 7.29 (t, J=7.96 Hz, 1H), 7.15 (d, J=7.58 Hz, 1H), 6.66 (d, J=5.31 Hz, 1H), 4.28-4.19 (m, 2H), 3.82 (t, J=5.31 Hz, 2H) 3.64-3.57 (m, 2H), 3.41-3.33 (m, 2H), 3.02-3.03 (m, 2H) 2.94 (s, 6H), 2.02-2.00 (m, 2H), 1.88-1.85 (m, 2H); ESI MS (m/z) 527: LCMS retention time t_{R}=1.30 min: analytical HPLC t_{R}=1.84 min.

### Example 51

### N′-(4-{1-ethyl-4-[2-({3-[2-(4-morpholinyl)ethyl]phenyl}amino)-4-pyrimidinyl]-1H-pyrazol-3-yl}phenyl)-N,N-dimethylurea

¹H NMR (400 MHz, DMSO-d₆) δ ppm 10.9 (bs, 1H), 9.75 (s, 1H), 8.43 (s, 1H), 8.35 (s, 1H), 8.32 (d, J=5.4 Hz, 2H), 7.70 (m, 1H), 7.60 (s, 1H), 7.52 (d, J=8.8 Hz, 2H), 7.49 (d, J=7.9 Hz, 1H), 7.38 (d, J=8.8 Hz, 2H), 7.19 (t, J=7.9 Hz, 1H), 6.85 (d, J=7.9 Hz, 1H), 6.66 (d, J=5.4 Hz, 2H), 4.23 (q, J=7.3 Hz, 2H), 3.98 (m, 2H), 3.79 (m, 2H), 3.50 (m, 2H), 1.76 (m, 2H), 1.46 (t, J=7.3 Hz, 3H), 0.88 (m, 2H). ESI MS (m/z) 541; HPLC (Method A) t_{R}=5.72 min.

### Example 52

### N,N-diethyl-N′-{4-[1-methyl-4-(2-{[3-(1-pyrrolidinylmethyl)phenyl]amino}-4-pyrimidinyl)-1H-pyrazol-3-yl]phenyl}urea

¹H NMR (400 MHz, DMSO-d₆) δ ppm 9.46 (s, 1H), 8.29 (d, J=5.1 Hz, 1H), 8.25 (s, 1H), 7.68 (s, 1H), 7.56 (d, J=8.8 Hz, 1H), 7.55 (d, J=8.8 Hz, 2H), 7.37 (d, J=8.8 Hz, 2H), 7.14 (t, J=7.8 Hz, 1H), 6.86 (d, J=7.8 Hz, 1H), 6.59 (d, J=5.1 Hz, 1H), 3.94 (s, 3H), 3.58-3.66 (m, 2H), 3.50-3.58 (m, 2H), 3.39-3.33 (m, 2H), 3.36 (q, J=6.91 Hz, 4H), 1.74-1.81 (m, 2H), 1.67-1.73 (m, 3H), 1.10 (t, J=7.1 Hz, 6H); ESI MS (m/z) 525; HPLC (Method A but with gradient 5-95 over 5 min) t_{R}=2.22 min.

### Example 53

### N,N-dimethyl-N′-{4-[1-methyl-4-(2-{[3-(1-pyrrolidinylmethyl)phenyl]amino}-4-pyrimidinyl)-1H-pyrazol-3-yl]phenyl}urea

¹H NMR (400 MHz, DMSO-d₆) δ ppm 9.62 (bs, 1H), 8.33 (d, J=5.3 Hz, 1H), 8.30 (s, 1H), 8.14 (s, 1H), 7.83 (s, 1H), 7.62 (d, J=7.8 Hz, 1H) 7.52 (d, J=8.6 Hz, 2H), 7.38 (d, J=8.6 Hz, 2H), 7.28 (t, J=7.8 Hz, 1H), 7.16 (d, J=7.8 Hz, 1H), 6.65 (d, J=5.3 Hz, 1H), 4.21 (d, J=5.3 Hz, 2H), 3.94 (m, 3H), 3.29-3.41 (m, 2H), 3.17 (s, 3H), 2.98-3.10 (m, 2H), 2.94 (s, 3H), 1.94-2.09 (m, 2H), 1.80-1.93 (m, 2H); ESI MS (m/z) 497; HPLC (Method A but with gradient 5-95 over 5 min) t_{R}=2.14 min.

### Intermediate 14

### 3-(4-nitrophenyl)-1H-pyrazole

In a 1000 ml flask under argon was dissolved 4-nitroacetaphenone (30.0 g, 0.182 mol) in 300 ml dry DMF. To this solution was added DMF-DMA (29.1 ml, 0.218 mol) and heat at 80° C. for 2 hours, after which time the reaction was concentrated to dryness under vacuum. The resulting dark solid was dissolved in 300 ml absolute ETOH and Hydrazine monohydrate (28.3 ml, 0.582 mol) was added. The resulting solution was heated at 75° C. for 1.5 hours, at which time the reaction was cooled to room temperature and poured onto 1500 ml ice water. The resulting yellow precipitate was filtered, washed with 2000 ml water, and dried under vacuum to yield 3-(4-nitrophenyl)-1H-pyrazole (31.6 g, 70% purity). This material was used as is in the next step.

### Intermediate 15

### 1-methyl-3-(4-nitrophenyl)-1H-pyrazole

In a 1000 ml flask under argon was dissolved 3-(4-nitrophenyl)-1H-pyrazole (31.6 g, 0.167 mol) in 300 ml dry DMF. To this solution was added cesium carbonate (65.3 g, 0.200 mol) followed by iodomethane (22 ml, 0.351 mol). The reaction was stirred at room temperature overnight, after which time an additional 2 ml iodomethane was added to force reaction to completion. The reaction was carefully diluted with 600 ml water and the resulting tan solids were filtered, washed with 1500 ml water, 500 ml hexanes, and dried under vacuum to yield 1-methyl-3-(4-nitrophenyl)-1H-pyrazole (22.8 g, >95% pure).

### Intermediate 16

### 4-bromo-1-methyl-3-(4-nitrophenyl)-1H-pyrazole

In a 1000 ml flask under argon was dissolved 1-methyl-3-(4-nitrophenyl)-1H-pyrazole (22.8 g, 0.112 mol) in 450 ml chloroform. To this solution was added bromine (8.7 ml, 0.169 mol) over 5 minutes with rapid stirring at room temperature, resulting in an orange precipitate. After 20 minutes, the mixture was poured into 1000 ml EtOAc (heterogeneous mixture) and washed with saturated aqueous 50/50 NaHCO3/Na2S2O3 (2×700 ml). The now homogeneous organic phase was then washed with brine, dried over Na2SO4, and concentrated under vacuum to ˜20% overall volume. The solution was diluted with 1000 ml hexanes resulting in the precipitation of 4-bromo-1-methyl-3-(4-nitrophenyl)-1H-pyrazole (29.0 g, >95% pure). ¹H NMR (500 MHz, DMSO-d₆) δ 9.34-8.31 (m, 2H), 8.14-8.12 (m, 3H), 3.93 (s, 3H).

### Intermediate 17

### 1-methyl-3-(4-nitrophenyl)-4-(4,4,5,5-tetramethyl-1,3,2-dioxaborolan-2-yl)-1H-pyrazole

In a 1000 ml flask fitted with a condenser was placed potassium acetate (31.2 g, 0.318 mol) which was then dried under hi-vac at 50° C. overnight. The following morning, 4-bromo-1-methyl-3-(4-nitrophenyl)-1H-pyrazole (30.0 g, 0.106 mol), bis(pinacolato)-diboron (29.7 g, 0.117 mol), and 250 ml 1,4-dioxane were added. The mixture was deoxygenated with bubbling nitrogen for 15 minutes. After adding dichloro-bis(triphenylphosphine)palladium(II) (3.72 g, 5.30 mmol) the reaction mixture was heated at 95° C. under argon for 3 hours, after which the reaction was concentrated under vacuum. The resulting brown solid was dissolved in 550 ml hot EtOH and treated with activated carbon for 30 minutes after which time it was hot filtered through a pad of Celite 545. The filtered solution was placed in a −20° C. freezer overnight, resulting in the crystallization of 1-methyl-3-(4-nitrophenyl)-4-(4,4,5,5-tetramethyl-1,3,2-dioxaborolan-2-yl)-1H-pyrazole (17.3 g, 89% pure). Further crystallizations of the mother liquor were unsuccessful, however reverse-phase prep HPLC (MeCN/H₂O—C18) was able to isolate and additional 4.5 g of desired product. ESI MS m/z 330 [C₁₆H₂₀BN₃O₄+H]⁺.

The primary byproduct of this reaction is 1-methyl-3-(4-nitrophenyl)-1H-pyrazole.

### Intermediate 18

### 2-chloro-4-[1-methyl-3-(4-nitrophenyl)-1H-pyrazol-4-yl]pyrimidine

A mixture of 17 (17 g, 52 mmol), 2,4-dichloropyrimidine (12 g, 78 mmol), and sodium carbonate (7.1 g, 67 mmol) in water (39 mL) and ethanol (200 mL) was degassed with argon for 30 minutes. Trans-dichlorobis (triphenylphosphine)palladium (II) (1.8 g, 2.6 mmol) was added and the slurry stirred vigorously under argon at 75° C. for 16 h. The solids formed in the reaction mixture were filtered and dissolved in hot tetrahydrofuran (2 L). This tetrahydrofuran solution was concentrated to 500 mL and the resulting slurry was allowed to sit overnight. The slurry was filtered to give 18 (6.7 g, 24% over 2 steps) as a tan solid: ESI MS m/z 316 [C₁₄H₁₀ClN₅O₂+H]⁺.

### Intermediate 19

### 4-bromo-3-(4-nitrophenyl)-1H-pyrazole

A solution of Intermediate 14 (595 mmol) in DMF (1 L) was treated with N-bromo succinimide (654 mmol). The reaction was stirred for 30 min at room temperature and poured into ice-water (1 L). The product precipitated out of solution and was filtered, washed with water (4×500 mL) and dried to provide Intermediate 19 as an off-white powder (90%). ESMS [M+H]+=269.2.

### Intermediate 20

### 2-[4-bromo-3-(4-nitrophenyl)-1H-pyrazol-1-yl]ethanol

To a mixture of sodium hydride (60% dispersion in mineral oil, 10 g, 250 mmol) in N,N-dimethylformamide (250 mL) was added a solution of Intermediate 19 (57 g, 210 mmol) in N,N-dimethylformamide (250 mL) via addition funnel over 45 minutes. After stirring for an additional 30 min, 2-bromoethanol (18 mL, 250 mmol) was added dropwise. The solution was stirred at room temperature for 16 h. The reaction was quenched by the addition of saturated NH₄Cl (100 mL) and EtOAc (300 mL). The organic layer was washed with aqueous 5% lithium chloride solution (2×100 mL) and water (3×100 mL). The organic layer was dried over sodium sulfate, filtered, and concentrated under reduced pressure. The residue was suspended in methylene chloride (200 mL) and the solids were filtered and suspended in 1:1 Hexanes/ethyl acetate (200 mL). After stirring the precipitate for 3 h the solids were filtered and dried to obtain 15 (24 g, 37%) as a tan solid. The filtrates were combined and purified by chromatography (silica, 0-30% ethyl acetate/methylene chloride). The product was obtained as a mixture of regioisomers and was suspended in 1:1 hexanes/ethyl acetate (50 mL) and stirred to 30 min, filtered and dried to obtain Intermediate 20 (6 g, 9%) as a tan solid: ¹H NMR (500 MHz, DMSO-d₆) δ 8.34-8.31 (m, 2H), 8.16-8.12 (m, 3H), 4.98 (t, J=5.0 Hz, 1H), 4.23 (t, J=5 Hz, 2H), 3.79 (t, J=5 Hz, 2H), 3.32 (s, 3H).

### Intermediate 21

### 2-[3-(4-nitrophenyl)-4-(4,4,5,5-tetramethyl-1,3,2-dioxaborolan-2-yl)-1H-pyrazol-1-yl]ethanol

A mixture of Intermediate 20 (30 g, 96 mmol), bis(pinacolato)diboron (49 g, 190 mmol), and potassium acetate (27 g, 280 mmol) in 1,4-dioxane (1000 mL) was degassed with argon for 30 min followed by the addition of trans-dichloro-bis(triphenylphosphine)-palladium (II) (3.4 g, 4.8 mmol). The reaction mixture was stirred at 100° C. for 16 h. The crude reaction was filtered through diatomaceous earth and the filtrate was concentrated and purified by MPLC (silica, 0-50% ethyl acetate/methylene chloride) to afford a 1:1 mixture of Intermediate 21 and the dehalogenated side product (29 g) as analyzed by LCMS: ESI MS m/z 360 [C₁₇H₂₂BN₃O₅+H]⁺.

### Intermediate 22

### 2-[4-(2-chloro-4-pyrimidinyl)-3-(4-nitrophenyl)-1H-pyrazol-1-yl]ethanol

To a solution of Intermediate 21 and the dehalogenated side product (29 g) in ethanol (350 mL) was added 2,4-dichloropyrimidine (24 g, 162 mmol), trans-dichlorobis(triphenylphosphine)palladium (II) (1.82 g, 2.60 mmol) and a solution of sodium carbonate (17 g, 162 mmol) in water (50 mL). The resulting reaction mixture was stirred at 80° C. for 16 h. The reaction was cooled and filtered and the filtrate was concentrated and purified by MPLC (silica, 0-50% ethyl acetate/methylene chloride to elute the dehalogenated side product and then 5% methanol/methylene chloride containing 1% ammonium hydroxide) to give Intermediate 22 (5.4 g, 16% over 2 steps) as an oil: ESI MS m/z 346 [C₁₅H₁₂ClN₅O₃+H]⁺.

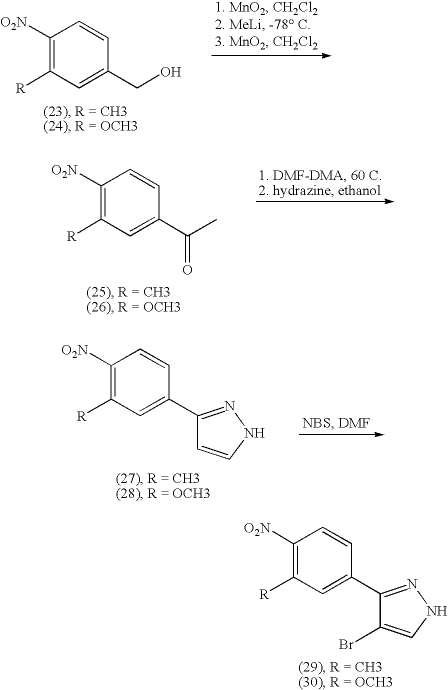

### Intermediates 25 & 26

To a stirred solution of Intermediate 23 or 24 (60 mmol) in methylene chloride (200 mL) was added manganese oxide (52 g, 600 mmol). The reaction mixture was stirred at room temperature for 2 days and filtered through diatomaceous. The filter cake was washed with methylene chloride (500 mL) and the filtrate was concentrated under reduced pressure to afford the crude aldehyde. To a solution of the aldehyde (8.7 g, 54 mmol) at −78° C. in tetrahydrofuran (180 mL) was added MeLi (2 M in THF, 80 mmol, 40 mL) dropwise via addition funnel. The resulting solution was stirred under nitrogen, at −78° C., for 4 hours. The reaction mixture was quenched slowly with saturated ammonium chloride solution at −78° C. and warmed to 0° C. The mixture was partitioned between ethyl acetate (500 mL) and water (300 mL). The organic layer was separated, dried over sodium sulfate and concentrated under reduced pressure. The crude oil was purified by chromatography (silica gel, 2:1 hexanes/ethyl acetate) to afford the alcohol intermediate. To a stirred solution of the alcohol (4.0 g, 22 mmol) in methylene chloride (75 mL) was added manganese oxide (26 g, 300 mmol). The reaction mixture was stirred at room temperature for 2 days and then filtered through diatomaceous. The filter cake was washed with methylene chloride (500 mL) and the filtrate was concentrated under reduced pressure and the resulting solid was purified by chromatography (silica gel, 4:1 hexanes/ethyl acetate).

Intermediate 25 (4.3 g, 35% for 3 steps): ¹H NMR (500 MHz, CDCl₃) [symbol] 8.01-7.99 (m, 1H), 7.91 (s, 1H), 7.89-7.82 (m, 1H), 2.65-2.64 (m, 6H); HPLC >99%, t_{R}=8.05 min;

Intermediate 26 (1.6 g, 24% for 3 steps): ¹H NMR (500 MHz, CDCl₃) [symbol] 7.90-7.88 (m, 1H), 7.68 (s, 1H), 7.53-7.55 (m, 1H), 4.01 (s, 3H), 2.65 (s, 3H).

### Intermediates 27 & 28

A slurry of Intermediate 25 or 26 (23 mmol) in N,N-dimethylformamide dimethylacetal (56 g, 470 mmol) was stirred at 60° C. for 3 h and concentrated under reduced pressure. The crude residue was dissolved in ethanol (32 mL), cooled to 0° C., and hydrazine monohydrate (5.9 g, 120 mmol) was added dropwise while maintaining the reaction temperature below 10° C. Once the addition was complete the ice bath was removed and the reaction mixture was stirred at room temperature for 1.5 h. The reaction was cooled to 0° C., quenched with water (30 mL), and extracted with ethyl acetate (250 mL). The organic layer was dried over sodium sulfate and concentrated under reduced pressure. The crude residue was purified by chromatography (silica gel, 2:1 hexanes/ethyl acetate).

Intermediate 27 (2.0 g, 42%) as an oil: ¹H NMR (500 MHz, CDCl₃) δ 10.80 (bs, 1H), 8.07-8.06 (m, 1H), 7.79-7.74 (m, 2H), 7.67-7.66 (m, 1H), 6.73-6.72 (m, 1H), 2.67 (s, 3H)

Intermediate 28 (1.4 g, 78%) as a yellow solid: it was carried crude to next step.

### Intermediates 29 & 30

To a solution of Intermediate 27 or 28 (9.8 mmol) in N,N-dimethylformamide (20 mL) at ° C. was added N-bromo succinimide (2.3 g, 13 mmol). The resulting solution was stirred at room temperature for 14 h. The reaction mixture was quenched with water (100 mL), stirred for 0.5 h, and the resulting precipitate was collected by filtration.

Intermediate 29: 2.5 g, 89%; ESI MS m/z 282/284 [C₁₀H₈BrN₃O₂+H]⁺

Intermediate 30: 1.7 g, 89%

Intermediates 29 & 30 were converted to the corresponding boronates according to the procedure established for Intermediate 17.

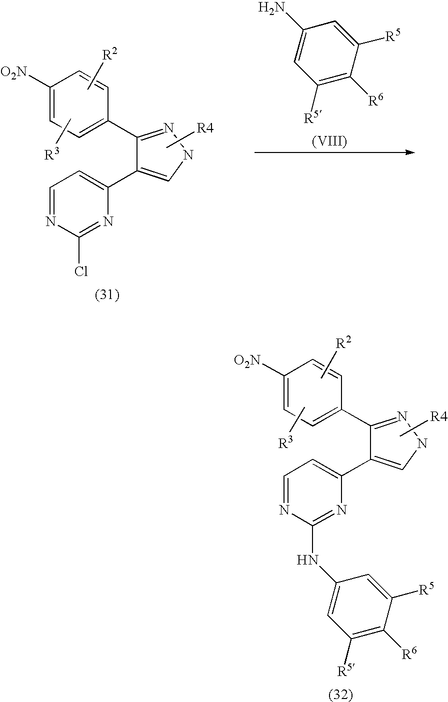

### Intermediate 32

To a solution of pyrazole 31 (1 equiv.) and the substituted aniline (1.2-1.5 equiv.) in 2-propanol or 1-pentanol (0.25-0.15 M) at 70° C. was added 6 N HCl in 2-propanol (1.2 equiv) and the reaction mixture was heated at 85° C. (2-propanol) or 140° C. (1-pentanol) until the reaction was determined to be complete by LCMS. In certain cases a small quantity of DMSO was added to the reaction if the starting materials were not soluble. The reaction mixture was concentrated under reduced pressure and the residue was dissolved in ethyl acetate and washed with saturated ammonium chloride solution. The organic phase was dried over Na₂SO₄, filtered, concentrated and purified by flash chromatography (silica, 0-20% methanol/methylene chloride) to afford Intermediate 32.

The intermediates 32(a)-32(l) were prepared according the procedure outlined for Intermediate 32:

### Intermediate 32(a)

### 2-{4-[2-[(3-fluorophenyl)amino]-4-pyrimidinyl}-3-(4-nitrophenyl)-1H-pyrazol-1-yl]ethanol

Yield 86%, ESI MS m/z 421 [C₂₁H₁₇FN₆O₃+H]⁺.

### Intermediate 32(b)

### 2-[3-(4-nitrophenyl)-4-(2-{[3-(1-pyrrolidinylmethyl)phenyl]amino}-4-pyrimidinyl)-1H-pyrazol-1-yl]ethanol

Yield 52%, ¹H NMR (400 MHz, MeOD-d₄) δ 8.67 (s, 1H), 8.34 (m, 1H), 8.03 (d, J=8.6 Hz, 2H), 7.75 (d, J=8.6 Hz, 2H), 7.53 (m, 1H), 7.35 (m, 1H), 7.28 (m, 1H), 7.17 (m, 2H), 4.41 (t, J=5.3 Hz, 2H), 4.29 (s, 2H), 4.02 (t, J=5.3 Hz, 2H), 3.52 (m, 4H), 3.17 (m, 4H); ESI MS m/z 486 [C₂₆H₂₇FN₇O₃+H]⁺.

### Intermediate 32(c)

### 2-[4-[2-({3-[2-(4-morpholinyl)ethyl]phenyl}amino)-4-pyrimidinyl]-3-(4-nitrophenyl)-1H-pyrazol-1-yl]ethanol

Yield 97%, ESI MS m/z 516 [C₂₇H₂₉N₇O₄+H]⁺.

### Intermediate 32(d)

### 2-{3-(3-methyl-4-nitrophenyl)-4-[2-({3-[2-(4-morpholinyl)ethyl]phenyl}amino)-4-pyrimidinyl]-1H-pyrazol-1-yl}ethanol

Yield 66%, ESI MS m/z 530 [C₂₈H₃₁N₇O₄+H]⁺.

### Intermediate 32(e)

### 2-{3-[3-(methyloxy)-4-nitrophenyl]-4-[2-({3-[2-(4-morpholinyl)ethyl]phenyl}amino)-4-pyrimidinyl]-1H-pyrazol-1-yl}ethanol

Yield 83%, ESI MS m/z 546 [C₂₈H₃₁N₇O₅+H]⁺.

### Intermediate 32(f)

### 2-[4-[2-({3-[(4-methyl-1-piperazinyl)methyl]phenyl}amino)-4-pyrimidinyl]-3-(4-nitrophenyl)-1H-pyrazol-1-yl]ethanol

Yield 55%, ESI MS m/z 515 [C₂₇H₃₀N₈O₃+H]⁺.

### Intermediate 32(g)

### 2-[4-(2-{[3-(4-methyl-1-piperazinyl)phenyl]amino}-4-pyrimidinyl)-3-(4-nitrophenyl)-1H-pyrazol-1-yl]ethanol

Yield 65%, ESI MS m/z 501 [C₂₆H₂₈N₈O₃+H]⁺.

### Intermediate 32(h)

### 2-{4-[3-({4-[1-methyl-3-(4-nitrophenyl)-1H-pyrazol-4-yl]-2-pyrimidinyl}amino)phenyl]-1-piperazinyl}ethanol

Yield 52%, ¹H NMR (400 MHz, DMSO-d₆) δ 9.28 (s, 1H), 8.40-8.37 (m, 2H), 8.21-8.14 (m, 2H), 7.86-7.81 (m, 2H), 7.17 (s, 1H), 6.98-6.95 (m, 1H), 6.84 (t, J=8.08 Hz, 1H), 6.78 (d, J=5.31 Hz, 1H), 6.45 (d, J=2.27 Hz, 1H), 4.46-4.443 (m, 1H), 3.99 (s, 3H), 2.55-2.52 (s, 2H), 3.18-3.16 (m, 2H), 3.09-2.91 (m, 4H), 2.50-2.33 (m, 4H). ESI MS m/z 501 [C₂₆H₂₈N₈O₃+H]⁺; analytical HPLC t_{R}=2.15 min.

### Intermediate 32(i)

### 4-[1-methyl-3-(4-nitrophenyl)-1H-pyrazol-4-yl]-N-[3-(1-pyrrolidinylmethyl)phenyl]-2-pyrimidinamine

Yield 71%, ESI MS m/z 456 [C₂₅H₂₅N₇O₂+H]⁺.

### Intermediate 32(j)

### 2-(4-{[3-({4-[1-methyl-3-(4-nitrophenyl)-1H-pyrazol-4-yl]-2-pyrimidinyl}amino)phenyl]methyl}-1-piperazinyl)ethanol

Yield 46%, ESI MS m/z 515 [C₂₇H₃₀N₈O₃+H]⁺.

### Intermediate 32(k)

### 4-[1-methyl-3-(4-nitrophenyl)-1H-pyrazol-4-yl]-N-[3-(4-methyl-1-piperazinyl)phenyl]-2-pyrimidinamine

Yield 83%, ¹H NMR (400 MHz, DMSO-d₆) δ 9.27 (s, 1H), 8.38 (d, J=5.2 Hz, 1H), 8.37 (s, 1H), 8.18 (d, J=9.2 Hz, 2H), 7.83 (d, J=9.2 Hz, 2H), 7.17 (s, 1H), 6.95 (d, J=8.3 Hz, 1H), 6.82 (t, J=8.3 Hz, 1H), 6.78 (d, J=5.2 Hz, 1H), 6.45 (d, J=8.3 Hz, 1H), 3.99 (s, 3H), 3.00 (m, 4H), 2.41 (m, 4H), 2.21 (s, 3H); ESI MS m/z 471 [C₂₅H₂₆N₈O₂+H]⁺.

### Intermediate 32(l)

### 4-[1-methyl-3-(4-nitrophenyl)-1H-pyrazol-4-yl]-N-{3-[2-(4-morpholinyl)ethyl]phenyl}-2-pyrimidinamine

Yield 74%, ESI MS m/z 486 [C₂₆H₂₇N₇O₃+H]⁺.

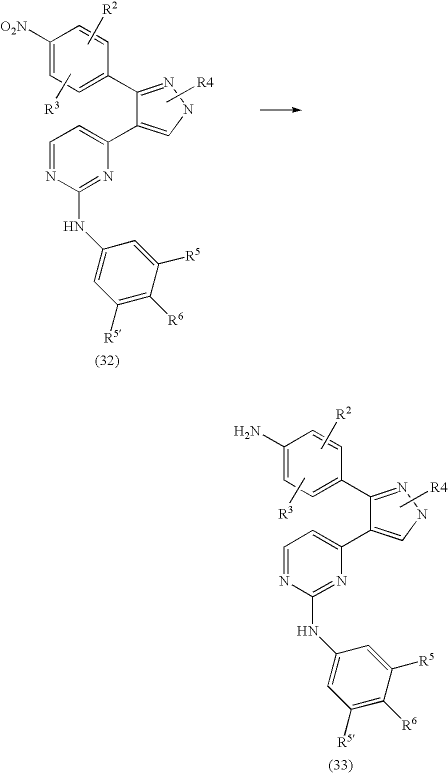

### Intermediate 33

Intermediate 33 may be obtained from Intermediate 32 by reduction of the nitro group.

### Method A

To a solution of Intermediate 32 (1 equiv) in 1:1 6N HCl/ethanol (25 mL/g of substrate) was added tin (5 equiv) and the mixture was heated at 70° C. for 1 h. The reaction mixture was filtered to remove the solids and the filtrate was concentrated. The crude residue was suspended in ethyl acetate (500 mL/g of substrate) and 2 N NaOH (300 mL/g of substrate) and stirred vigorously for 2 h. The reaction mixture was filtered through diatomaceous earth and the biphasic filtrate was separated. The aqueous phase was extracted with ethyl acetate and the combined organic phases were washed with water and brine, dried over Na₂SO₄, filtrated and concentrated under reduced pressure to afford Intermediate 33.

### Method B

To a solution of Intermediate 32 (2.0 mmol) and copper (I) chloride (3.5 g, 35 mmol) in anhydrous tetrahydrofuran (10 mL) and anhydrous methanol (10 mL) was added KBH₄ (2.3 g, 41 mmol) portion wise. The mixture evolved gas and after 15 min was heated at 70° C. under a nitrogen atmosphere for 18 h. The mixture was cooled, diluted with 1:1 methanol/water (200 mL each) and filtered through a pad of diatomaceous earth. The filtrate was concentrated under reduced pressure and purified via chromatography (silica, 0-20% CMA/methylene chloride) to obtain Intermediate 33.

The intermediates 33(a)-33(l) were prepared according the procedure outlined for Intermediate 33:

### Intermediate 33(a)

### 2-[4-{2-[(3-fluorophenyl)amino]-4-pyrimidinyl}-3-(4-aminophenyl)-1H-pyrazol-1-yl]ethanol

Yield 99%, ESI MS m/z 391 [C₂₁H₁₉FN₆O+H]⁺.

### Intermediate 33(b)

### 2-[3-(4-aminophenyl)-4-(2-{[3-(1-pyrrolidinylmethyl)phenyl]amino}-4-pyrimidinyl)-1H-pyrazol-1-yl]ethanol

Yield 99%, ¹H NMR (400 MHz, MeOD-d₄) δ 8.22 (s, 1H), 8.14 (d, J=5.2 Hz, 1H), 7.62 (s, 1H), 7.57 (d, J=7.6 Hz, 1H), 7.25 (d, J=8.6 Hz, 2H), 7.22 (t, J=7.6 Hz, 1H), 6.96 (d, J=7.6 Hz, 1H), 6.75 (d, J=8.6 Hz, 2H), 6.61 (d, J=5.2 Hz, 1H), 4.24 (t, J=5.3 Hz, 2H), 3.95 (t, J=5.3 Hz, 2H), 3.64 (s, 2H), 2.62 (m, 4H), 1.81 (m, 4H); ESI MS m/z 456 [C₂₆H₂₉FN₇O+H]⁺.

### Intermediate 33(c)

### 2-[4-[2-({3-[2-(4-morpholinyl)ethyl]phenyl}amino)-4-pyrimidinyl]-3-(4-aminophenyl)-1H-pyrazol-1-yl]ethanol

Yield 97%, ESI MS m/z 486 [C₂₇H₃₁N₇O₂+H]⁺.

### Intermediate 33(d)

### 2-{3-(3-methyl-4-aminophenyl)-4-[2-({3-[2-(4-morpholinyl)ethyl]phenyl}amino)-4-pyrimidinyl]-1H-pyrazol-1-yl}ethanol

Yield 99%, ESI MS m/z 500 [C₂₈H₃₃N₇O₂+H]⁺.

### Intermediate 33(e)

### 2-{3-[3-(methyloxy)-4-aminophenyl]-4-[2-({3-[2-(4-morpholinyl ethyl]phenyl}amino)-4-pyrimidinyl]-1H-pyrazol-1-yl}ethanol

Yield 68%, ESI MS m/z 516 [C₂₈H₃₃N₇O₃+H]⁺.

### Intermediate 33(f)

### 2-[4-[2-({3-[(4-methyl-1-piperazinyl)methyl]phenyl}amino)-4-pyrimidinyl]-3-(4-aminophenyl)-1H-pyrazol-1-yl]ethanol

Yield 71%, ESI MS m/z 485 [C₂₇H₃₂N₈O+H]⁺.

### Intermediate 33(g)

### 2-[4-(2-{[3-(4-methyl-1-piperazinyl)phenyl]amino}-4-pyrimidinyl)-3-(4-aminophenyl)-1H-pyrazol-1-yl]ethanol

Yield 82%, ESI MS m/z 471 [C₂₆H₃₀N₈O+H]⁺.

### Intermediate 33(h)

### 2-{4-[3-({4-[1-methyl-3-(4-aminophenyl)-1H-pyrazol-4-yl]-2-pyrimidinyl}amino)phenyl]-1-piperazinyl}ethanol

Yield 75%, ESI MS m/z 471 [C₂₆H₃₀N₈O+H]⁺; analytical HPLC t_{R}=1.61 min.

### Intermediate 33(i)

### 4-[1-methyl-3-(4-aminophenyl)-1H-pyrazol-4-yl]-N-[3-(1-pyrrolidinylmethyl)phenyl]-2-pyrimidinamine

Yield 67%, ESI MS m/z 426 [C₂₅H₂₇N₇+H]⁺.

### Intermediate 33(j)

### 2-(4-{[3-({4-[1-methyl-3-(4-aminophenyl)-1H-pyrazol-4-yl]-2-pyrimidinyl}amino)phenyl]methyl}-1-piperazinyl)ethanol

Yield 92%, ESI MS m/z 485 [C₂₇H₃₃N₈O₃+H]⁺.

### Intermediate 33(k)

### 4-[1-methyl-3-(4-aminophenyl)-1H-pyrazol-4-yl]-N-[3-(4-methyl-1-piperazinyl)phenyl]-2-pyrimidinamine

Yield 52%, ¹H NMR (400 MHz, DMSO-d₆) δ 9.30 (s, 1H), 8.27 (d, J=5.3 Hz, 1H), 8.22 (s, 1H), 7.54 (d, J=8.6 Hz, 2H), 7.42 (s, 1H), 7.36 (d, J=8.6 Hz, 1H), 7.18 (d, J=8.1 Hz, 1H), 7.04 (t, J=8.1 Hz, 1H), 6.54 (d, J=5.3 Hz, 1H), 6.45 (d, J=8.1 Hz, 1H), 3.94 (s, 3H), 3.08 (m, 4H), 2.45 (m, 4H), 2.22 (s, 3H); ESI MS m/z 441 [C₂₅H₂₈N₈+H]⁺.

### Intermediate 33(l)

### 4-[1-methyl-3-(4-nitrophenyl)-1H-pyrazol-4-yl]-N-{3-[2-(4-morpholinyl)ethyl]phenyl}-2-pyrimidinamine

Yield 84%, ESI MS m/z 456 [C₂₆H₂₉N₇O+H]⁺.

### General Synthesis of Urea Targets From Intermediate 33.

Acylation of Intermediate 33 using the appropriate method afforded the desired target compound (I):

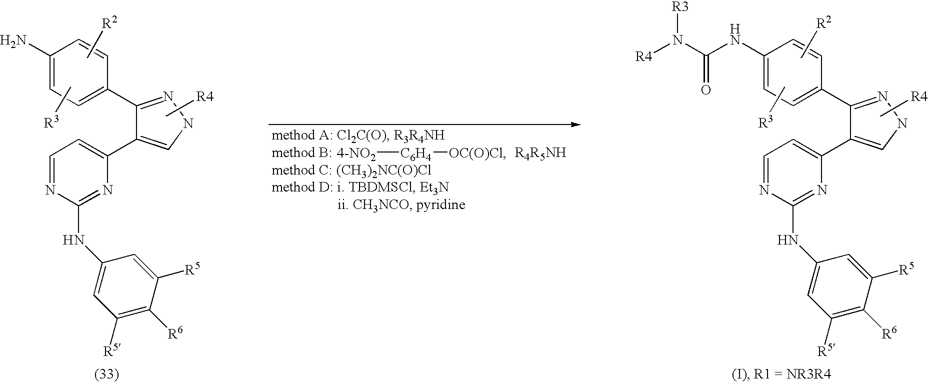

### Method A

To a solution of phosgene (1.7 M in toluene, 0.50 mL, 0.86 mmol) in THF (5 mL) at 0° C. was added Intermediate 33 (0.66 mmol). The resulting suspension was warmed to room temperature and stirred for 15 min. Diethyl amine (0.34 mL, 3.3 mmol) was added and the resulting mixture was stirred for 16 h. The reaction was quenched by the addition of saturated NH₄Cl (15 mL) and diluted with EtOAc (50 mL). The organics were dried over Na₂SO₄, concentrated and purified by MPLC (silica, 0-15% methanol/methylene chloride). The crude product was further purified by semi-preparatory HPLC (reverse phase silica, 15-90% methanol/NH₄OAc buffer) to afford the pure desired product which was dissolved in 5-6N HCl in IPA (2 mL) followed by trituration with diethyl ether (30 mL). The solids were filtered and lyophilized to afford desired product (I), where R¹═NR³R⁴.

### Method B

4-nitrophenylchloroformate (112 mg, 0.54 mmol) was added portion wise to a solution of Intermediate 33 (2.24 mmol) in methylene chloride (1.5 mL) and pyridine (1.5 mL) at 0° C. and stirred for 1 h. The formation of the carbamate intermediate was monitored by LCMS followed by the addition of pyrrolidine (0.5 mL). The reaction mixture was allowed to warm to rt and stirred for 18 h. The resulting yellow solution was poured into saturated sodium bicarbonate solution (50 mL) and extracted with methylene chloride (3×50 mL). The combined organic phases were washed with water (25 mL) and brine (25 mL), dried over Na₂SO₄, filtrated, concentrated and purified by MPLC to afford product (I), where R¹═NR³R⁴.

### Method C

To a solution of Intermediate 33 (0.51 mmol) in tetrahydrofuran (7.0 mL) was added dimethylcarbamyl chloride (2.2 g, 20 mmol). The reaction mixture was stirred at 45° C. for 3 days and concentrated under reduced pressure. The crude residue was purified by chromatography (silica gel, 94.5:5.0:0.5 methylene chloride/methanol/concentrated ammonium hydroxide) to afford the product (0.15 g) which was dissolved in iPrOH (3 mL) followed by dropwise addition of 5-6 N hydrochloric acid in iPrOH (0.10 mL). The mixture was stirred at room temperature for 15 min and concentrated to provide product (I), where R¹═NR³R⁴.

### Method D

(This method should be used for compounds that containing unprotected hydroxy groups in R4, R5, R5 or R6.)

Step 1: To a solution of Intermediate 33 (0.45 mmol) and imidazole (90 mg, 1.3 mmol) in N,N-dimethylformamide (3 mL) at 0° C. was added tert-butyl dimethylsilylchloride (0.15 g, 0.99 mmol) in one portion. The reaction mixture was stirred at 0° C. for 15 min and room temperature for 20 h. The reaction mixture was concentrated under reduced pressure and the residue was partitioned between ethyl acetate (20 mL) and water (10 mL). The organic layer was separated, dried over sodium sulfate, and concentrated under reduced pressure to provide the protected intermediate which was used crude in the next step.

Step 2: To a solution of the intermediate from step 1 (0.35 g, 0.59 mmol) in pyridine (6.0 mL) at 0° C. was added a 16% v/v solution of methyl isocyante in tetrahydrofuran (37 mg, 0.65 mmol). The resulting mixture was stirred, under nitrogen and at room temperature, for 18 h. The reaction mixture was concentrated under reduced pressure and the crude residue purified by chromatography (silica gel, 94.5:5.0:0.5 methylene chloride/methanol/concentrated ammonium hydroxide) to provide the methyl urea intermediate. To a solution of the methyl urea intermediate (0.21 g, 0.32 mmol) in ethanol (3 mL) was added 6N hydrochloric acid (4 mL). The resulting mixture was stirred at room temperature for 1.5 h and washed with diethyl ether (20 mL). The aqueous layer was separated, concentrated under reduced pressure, and the crude residue was purified by chromatography (silica gel, 94.5:5.0:0.5 methylene chloride/methanol/concentrated ammonium hydroxide) to provide the deprotected intermediate. The deprotected intermediate (0.28 mmol) was dissolved in a mixture of methanol (2 mL) and 2-PrOH (1 mL) and a 1M solution of hydrochloric acid in diethyl ether (0.55 mL) was added dropwise. The resulting mixture was stirred for 15 minutes and sonicated before concentrating under reduced pressure to afford product (I), where R¹═NR³R⁴.

### Example 54

### N-{4-[4-{2-[(3-fluorophenyl)amino]-4-pyrimidinyl}-1-(2-hydroxyethyl)-1H-pyrazol-3-yl]phenyl}-1-pyrrolidinecarboxamide

Method B, 33% as a white solid: mp 185-186° C.; ¹H NMR (500 MHz, DMSO-d₆) δ 9.72 (s, 1H), 8.33 (d, J=5.2 Hz, 1H), 8.25 (m, 1H), 8.18 (m, 1H), 7.68-65 (m, 1H), 7.57-56 (m, 2H), 7.46-44 (m, 1H), 7.36-35 (m 2H), 7.21-19 (m, 1H), 6.70-66 (m, 1H), 6.64-63 (m, 1H), 5.01-4.99 (m, 1H), 4.24-22 (m, 2H), 3.82-3.79 (m, 2H), 3.37-36 (m, 4H), 1.85 (m, 4H); ESI MS m/z 488 [C₂₆H₂₆FN₇O₂+H]⁺; HPLC t_{R}=12.46 min, 98.3% (AUC).

### Example 55

### N-cyclopropyl-N′-(4-{1-methyl-4-[2-({3-[2-(4-morpholinyl)ethyl]phenyl}amino)-4-pyrimidinyl]-1H-pyrazol-3-yl}phenyl)urea

Method A, 48% as an off-white solid: mp 143-145° C.; ¹H NMR (500 MHz, DMSO-d₆) δ 9.36 (s, 1H), 8.38 (s, 1H), 8.28 (d, J=5.2 Hz, 1H), 8.21 (s, 1H), 7.55 (s, 1H), 7.50 (d, J=8.0 Hz, 1H), 7.43 (d, J=8.7 Hz, 2H), 7.36 (d, J=8.7 Hz, 2H), 7.08 (t, J=7.8 Hz, 1H), 6.76 (d, J=7.5 Hz, 1H), 6.56 (d, J=5.1 Hz, 1H), 6.40 (d, J=2.6 Hz, 1H), 3.92 (s, 3H), 3.57 (s, 4H), 2.65 (t, J=8.0 Hz, 2H), 2.56-2.52 (m, 3H), 2.49-2.35 (m, 4H), 0.65-0.61 (m, 2H), 0.42-0.38 (m, 2H); ESI MS m/z 539 [C₃₀H₃₄N₈O₂+H]⁺; HPLC 98.6%, t_{R}=9.3 min.

### Example 56

### N-(4-{1-methyl-4-[2-({3-[2-(4-morpholinyl)ethyl]phenyl}amino)-4-pyrimidinyl]-1H-pyrazol-5-yl}phenyl)-1-pyrrolidinecarboxamide

Method A, off-white solid, mp=228-30° C., ¹H NMR (500 MHz, DMSO-d₆) δ 9.37 (s, 1H), 8.36 (m, 2H), 8.17 (d, 1H), 8.08 (s, 1H), 7.73 (d, 2H), 7.65 (s, 1H), 7.47 (d, 1H), 7.31 (d, 2H), 7.13 (t, 1H), 6.78 (d, 1H), 6.25 (d, 1H), 3.68 (s, 3H), 3.58 (m, 4H), 3.40 (m, 4H), 2.70 (m, 2H), 2.53 (br, 1H), 2.44 (br, 4H), 1.87 (br, 4H). ESI MS m/z 553 [M+H]⁺.

### Example 57

### N-(4-{1-(2-hydroxyethyl)-4-[2-({3-[2-(4-morpholinyl)ethyl]phenyl}amino)-4-pyrimidinyl]-1H-pyrazol-3-yl}phenyl)-1-pyrrolidinecarboxamide

Method A, 22% as an off-white solid: ¹H NMR (500 MHz, DMSO-d₆) δ 9.39 (s, 1H), 8.38 (bs, 1H), 8.28-8.27 (m, 1H), 8.23 (s, 1H), 8.19 (s, 1H), 7.59-7.56 (m, 3H), 7.51-7.49 (m, 1H), 7.37-7.35 (m, 2H), 7.09 (t, J=7.8 Hz, 1H), 6.77-6.76 (m, 1H), 6.56-6.55 (m, 1H), 4.23-4.21 (m, 2H), 3.82-3.80 (m, 2H), 3.58-3.56 (m, 4H), 3.39-3.36 (m, 6H), 2.67-2.63 (m, 2H), 2.50-2.47 (m, 2H), 2.41-2.36 (m, 4H), 1.87-1.84 (m, 4H); ESI MS m/z 583 [C₃₂H₃₈N₈O₃+H]⁺; HPLC 98.9% (AUC), t_{R}=9.33 min.

### Example 58

### N-cyclopropyl-N′-(4-{1-(2-hydroxyethyl)-4-[2-({3-[2-(4-morpholinyl)ethyl]phenyl}amino)-4-pyrimidinyl]-1H-pyrazol-3-yl}-2-methylphenyl)urea

Method A, 22% as a white solid: ¹H NMR (500 MHz, DMSO-d₆) δ 9.41 (s, 1H), 8.27-8.26 (m, 1H), 8.24 (s, 1H), 7.92-7.91 (m, 1H), 7.60 (s, 1H), 7.56 (m, 1H), 7.50-7.49 (m, 1H), 7.31-7.30 (m, 1H), 7.23-7.21 (m, 1H), 7.09-7.06 (m, 1H), 6.82-6.81 (m, 1H), 6.77-6.76 (m, 1H), 6.57-6.56 (m, 1H), 5.05-5.03 (m, 1H), 4.23-4.21 (m, 2H), 3.82-3.79 (m, 2H), 3.58-3.56 (m, 4H), 2.67-2.60 (m, 2H), 2.58-2.53 (m, 1H), 2.48-2.46 (m, 2H), 2.42-2.39 (m, 4H), 2.16 (s, 3H), 0.66-0.61 (m, 2H), 0.45-0.40 (m, 2H); ESI MS m/z 582 [C₃₂H₃₈N₈O₃+H]⁺; HPLC >99% (AUC), t_{R}=9.03 min.

### Example 59

### N,N-diethyl-N′-(4-{4-[2-({3-[4-(2-hydroxyethyl)-1-piperazinyl]phenyl}amino)-4-pyrimidinyl]-1-methyl-1H-pyrazol-3-yl}phenyl)urea

Method A, 34% as a yellow solid: ¹H NMR (500 MHz, DMSO-d₆) δ 10.37 (bs, 1H), 9.62 (bs, 1H), 8.31-8.26 (m, 3H), 7.56-7.54 (m, 2H), 7.44 (s, 1H), 7.38-7.36 (m, 2H), 7.19-7.17 (m, 1H), 7.13-7.10 (m, 1H), 6.63-6.60 (M, 2H), 3.95 (s, 3H), 3.83-3.81 (m, 2H), 3.70-3.68 (m, 2H), 3.61-3.59 (m, 2H), 3.38-3.33 (m, 4H), 3.26-3.10 (m, 6H), 1.09 (t, J=7.0 Hz, 6H); ESI MS m/z 570 [C₃₁H₃₉N₉O₂+H]⁺; HPLC 95.5% (AUC), t_{R}=9.86 min.

### Example 60

### N,N-diethyl-N′-{4-[1-methyl-4-(2-{[3-(4-methyl-1-piperazinyl)phenyl]amino}-4-pyrimidinyl)-1H-pyrazol-3-yl]phenyl}urea

Method A, 50% as a yellow solid: ¹H NMR (500 MHz, DMSO-d₆) δ 10.87 (bs, 1H), 9.66 (bs, 1H), 8.30-8.27 (m, 3H), 7.56-7.54 (m, 2H), 7.43 (s, 1H), 7.38-7.36 (m, 2H), 7.19-7.10 (m, 2H), 6.64-6.61 (m, 2H), 3.95-3.91 (m, 3H), 3.71-3.68 (m, 2H), 3.49-3.47 (m, 2H), 3.36 (q, J=14.0, 7.0 Hz, 4H), 3.17-3.05 (m, 4H), 2.81-2.78 (m, 3H), 1.09 (t, J=7.0 Hz, 6H); ESI MS m/z 540 [C₃₀H₃₇N₉O+H]⁺; HPLC 98.4% (AUC), t_{R}=9.83 min.

### Example 61

### N-ethyl-N′-(4-{1-methyl-4-[2-({3-[2-(4-morpholinyl)ethyl]phenyl}amino)-4-pyrimidinyl]-1H-pyrazol-3-yl}phenyl)urea

Method A, light brown solid, mp=139-41 C, ¹H NMR (500 MHz, DMSO-d₆) δ 9.35 (s, 1H), 8.50 (s, 1H), 8.25 (d, 1H), 8.20 (s, 1H), 7.55 (s, 1H), 7.50 (d, 1H), 7.40 (d, 2H), 7.35 (d, 2H), 7.05 (t, 1H), 6.85 (d, 1H), 6.55 (d, 1H), 6.10 (t, 1H), 3.95 (s, 3H), 3.55 (br, 4H), 3.10 (m, 2H), 2.65 (m, 2H), 2.40 (m, 4H), 1.03 (t, 3H). ESI MS m/z 527 [M+H]⁺.

### Example 62

### N,N-diethyl-N′-[4-(4-{2-[(3-{[4-(2-hydroxyethyl)-1-piperazinyl]methyl}phenyl)amino]-4-pyrimidinyl}-1-methyl-1H-pyrazol-3-yl)phenyl]urea

Method A, 39% as a yellow solid: ¹H NMR (500 MHz, DMSO-d₆) δ 11.14 (bs, 1H), 9.74 (bs, 1H), 8.32-8.28 (m, 3H), 7.82-7.81 (m, 1H), 7.67-7.66 (m, 1H), 7.56-7.54 (m, 2H), 7.37-7.35 (m, 2H), 7.31-7.28 (m, 1H), 7.26-7.22 (m, 1H), 6.65-6.64 (m, 1H), 4.26 (bs, 2H), 3.95 (s, 3H), 3.81-3.76 (m, 4H), 3.54-3.50 (m, 4H), 3.38-3.33 (m, 6H), 3.21-3.16 (m, 2H), 1.11-1.08 (m, 6H); ESI MS m/z 584 [C₃₂H₄₁N₉O₂+H]⁺; HPLC 96.7% (AUC), t_{R}=9.10 min.

### Example 63

### N-(4-{1-methyl-4-[2-({3-[2-(4-morpholinyl)ethyl]phenyl}amino)-4-pyrimidinyl]-1H-pyrazol-3-yl}phenyl)-1-pyrrolidinecarboxamide

Method A, 70% as a yellow solid: ¹H NMR (500 MHz, DMSO-d₆) δ 10.9 (bs, 1H), 9.61 (s, 1H), 8.30-8.27 (m, 2H), 8.23 (s, 1H), 7.64-7.63 (m, 1H), 7.57-7.55 (m, 2H), 7.51-7.50 (m, 1H), 7.38-7.36 (m, 2H), 7.19 (t, J=7.8 Hz, 1H), 6.84-6.82 (m, 1H), 6.62-6.61 (m, 1H), 4.00-3.95 (m, 5H), 3.81-3.77 (m, 2H), 3.50-3.48 (m, 2H), 3.39-3.36 (m, 4H), 3.31-3.23 (m, 2H), 3.14-3.06 (m, 2H), 3.00-2.96 (m, 2H), 1.87-1.84 (m, 4H); ESI MS m/z 553 [C₃₁H₃₆N₈O₂+H]⁺; HPLC 99.0% (AUC), t_{R}=9.43 min.

### Example 64

### N-ethyl-N′-(4-{1-methyl-4-[2-({3-[2-(4-morpholinyl)ethyl]phenyl}amino)-4-pyrimidinyl]-1H-pyrazol-3-yl}phenyl)urea

Method B, 22% as a light brown solid: mp 139-141° C.; ¹H NMR (500 MHz, DMSO-d₆) δ 9.37 (s, 1H), 8.50 (s, 1H), 8.28 (d, J=5.1 Hz, 1H), 8.21 (s, 1H), 7.55 (s, 1H), 7.50 (d, J=8.1 Hz, 1H), 7.43-7.41 (m, 2H), 7.36-7.34 (m, 2H), 7.09 (t, J=7.7 Hz, 1H), 6.76 (d, J=7.6 Hz, 1H), 6.57 (d, J=5.2 Hz, 1H), 6.11-6.10 (m, 1H), 3.93 (s, 3H), 3.57 (s, 4H), 3.12-3.10 (m, 2H), 2.66-2.64 (m, 2H), 2.53-2.49 (m, 2H), 2.43-2.41 (m, 4H), 1.07-1.04 (m, 3H); ESI MS m/z 527 [C₂₉H₃₄N₈O₂+H]⁺; HPLC 95.6%, t_{R}=9.0 min.

### Example 65

### N,N-dimethyl-N′-(4-{1-methyl-4-[2-({3-[2-(4-morpholinyl)ethyl]phenyl}amino)-4-pyrimidinyl]-1H-pyrazol-3-yl}phenyl)urea

Method B, 52% as a white powder: mp 132-134° C.; ¹H NMR (500 MHz, DMSO-d₆) δ 9.39 (s, 1H), 8.37 (s, 1H), 8.28 (d, J=5.2 Hz, 1H), 8.21 (s, 1H), 7.56-7.51 (m, 4H), 7.37-7.35 (m, 4H), 7.10-7.08 (m, 1H), 6.77 (d, J=7.6 Hz, 1H), 6.56 (d, J=5.2 Hz, 1H), 3.93 (s, 3H), 3.57 (t, J=4.6 Hz, 4H), 2.93 (s, 6H), 2.70-2.61 (m, 2H), 2.51-2.49 (m, 2H), 2.45-2.37 (m, 4H); ESI MS m/z 527 [C₂₉H₃₄N₈O₂+H]⁺; HPLC 98.6%, t_{R}=9.0 min.

### Example 66

### N-ethyl-N′-(4-{1-(2-hydroxyethyl)-4-[2-({3-[2-(4-morpholinyl)ethyl]phenyl}amino)-4-pyrimidinyl]-1H-pyrazol-3-yl}-2-methylphenyl)urea

Method A, 26% as a white solid: ¹H NMR (500 MHz, DMSO-d₆) δ 9.41 (s, 1H), 8.27-8.26 (m, 1H), 8.24 (s, 1H), 7.93-7.91 (m, 1H), 7.64-7.60 (m, 2H), 7.50-7.48 (m, 1H), 7.30 (s, 1H), 7.22-7.20 (m, 1H), 7.09-7.06 (m, 1H), 6.77-6.76 (m, 1H), 6.59-6.56 (m, 2H), 5.05-5.03 (m, 1H), 4.23-4.21 (m, 2H), 3.82-3.79 (m, 2H), 3.58-3.56 (m, 4H), 3.14-3.09 (m, 2H), 2.66-2.63 (m, 2H), 2.41-2.36 (m, 4H), 2.16 (s, 3H), 1.07 (t, J=7.1 Hz, 3H); ESI MS m/z 571 [C₃₁H₃₈N₈O₃+H]⁺; HPLC >99% (AUC), t_{R}=6.02 min.

### Example 67

### N,N-diethyl-N′-(4-{1-(2-hydroxyethyl)-4-[2-({3-[2-(4-morpholinyl)ethyl]phenyl}amino)-4-pyrimidinyl]-1H-pyrazol-3-yl}phenyl)urea

Method A, 38% as a white solid: ¹H NMR (500 MHz, DMSO-d₆) δ 9.39 (s, 1H), 8.29-8.27 (m, 1H), 8.24 (s, 2H), 7.59 (s, 1H), 7.55-7.54 (m, 2H), 7.51-7.49 (m, 1H), 7.38-7.36 (m, 2H), 7.09 (t, J=7.8 Hz, 1H), 6.77-6.75 (m, 1H), 6.58-6.57 (m, 1H), 4.24-4.22 (m, 2H), 3.82-3.80 (m, 2H), 3.58-3.56 (m, 4H), 3.37-3.33 (m, 5H), 2.67-2.64 (m, 2H), 2.50-2.47 (m, 2H), 2.41-2.36 (m, 4H), 1.09 (t, J=7.0 Hz, 6H); ESI MS m/z 585 [C₃₂H₄₀N₈O₃+H]⁺; HPLC >99% (AUC), t_{R}=9.70 min.

### Example 68

### N,N-diethyl-N′-{4-[1-(2-hydroxyethyl)-4-(2-{[3-(4-methyl-1-piperazinyl)phenyl]amino}-4-pyrimidinyl)-1H-pyrazol-3-yl]phenyl}urea

Method A, 22% as yellow solid: ¹H NMR (500 MHz, DMSO-d₆) δ 9.83 (s, 1H), 8.39-8.22 (m, 3H), 7.55 (d, J=7.0 Hz, 2H), 7.42-7.35 (m, 3H), 7.13 (d, J=8.0 Hz, 2H), 6.54-6.51 (m, 2H), 4.27-4.29 (m, 2H), 3.82-3.80 (m, 2H), 3.72-3.70 (m, 2H), 3.49-3.47 (m, 2H), 3.37 (q, J=7.5 Hz, 4H), 3.10-3.08 (m, 4H), 2.81-2.79 (bs, 3H), 1.09 (t, J=7.0 Hz, 6H); ESI MS m/z 570 [C₃₁H₃₉N₉O₂+H]⁺; HPLC 95.9%, t_{R}=9.4 min.

### Example 69

### N,N-diethyl-N′-(4-{1-(2-hydroxyethyl)-4-[2-({3-[(4-methyl-1-piperazinyl)methyl]phenyl}amino)-4-pyrimidinyl]-1H-pyrazol-3-yl}phenyl)urea

Method A, 33% as yellow solid: ¹H NMR (500 MHz, DMSO-d₆) [symbol] 9.92 (s, 1H), 8.39 (s, 1H), 8.3-8.29 (m, 2H), 7.89 (s, 1H), 7.63 (d, J=7.5 Hz, 1H), 7.57 (d, J=8.5 Hz, 2H), 7.38 (d, J=8.5 Hz, 2H), 7.32-7.29 (m, 2H), 6.69 (t, J=5.5 Hz, 1H), 4.30 (m, 2H), 4.26 (t, J=5.5 Hz, 2H), 3.82 (t, J=5.0 Hz, 2H), 3.65-3.46 (m, 8H), 3.37 (q, J=7.5 Hz, 4H), 2.80 (bs, 3H), 1.10 (t, J=7.0 Hz, 6H); ESI MS m/z 584 [C₃₂H₄₁N₉O₂+H]⁺; HPLC 97.2%, t_{R}=8.9 min.

### Example 70

### N-cyclopropyl-N′-[4-{1-(2-hydroxyethyl)-4-[2-({3-[2-(4-morpholinyl)ethyl]phenyl}amino)-4-pyrimidinyl]-1H-pyrazol-3-yl}-2-(methyloxy)phenyl]urea

Method A, 28% as yellow solid: ¹H NMR (500 MHz, DMSO-d₆) δ 9.42 (s, 1H), 8.29 (d, J=5.5 Hz, 1H), 8.25 (s, 1H), 8.17 (d, J=8.5 Hz, 1H), (s, 1H), 7.60 (s, 1H), 7.49 (d, J=8.5 Hz, 1H), 7.08-7.02 (m, 4H), 6.76 (d, J=7.5 Hz, 1H), 6.62 (d, J=5.5 Hz, 1H), 5.04 (t, J=5.0 Hz, 1H), 4.23 (t, J=5.0 Hz, 2H), 3.82-3.80 (m, 2H), 3.76 (s, 3H), 3.57 (t, J=4.5 Hz, 4H), 2.69-2.63 (m, 2H), 2.56-2.54 (m, 1H), 2.47-2.46 (m, 2H), 2.41-2.39 (m, 4H), 0.65-0.62 (m, 2H), 0.38 (m, 2H); ESI MS m/z 599 [C₃₂H₃₈N₈O₄+H]⁺; HPLC 97.5%, t_{R}=9.8 min.

### Example 71

### N-cyclopropyl-N′-(4-{1-(2-hydroxyethyl)-4-[2-({3-[2-(4-morpholinyl)ethyl]phenyl}amino)-4-pyrimidinyl]-1H-pyrazol-3-yl}phenyl)urea

Method A, 21% as yellow solid: ¹H NMR (300 MHz, DMSO-d₆) [symbol] 9.42 (s, 1H), 8.42 (s, 1H), 8.29-8.24 (m, 2H), 7.58-7.35 (m, 6H), 7.09 (t, J=6.0 Hz, 1H), 6.78 (d, J=9.0 Hz, 1H), 6.57 (d, J=6.0 Hz, 1H), 6.43-6.41 (m, 1H), 5.05 (m, 1H), 4.24-4.19 (m, 2H), 3.82-3.79 (m, 2H), 3.59-3.57 (m, 4H), 2.68-2.42 (m, 9H), 0.67-0.62 (m, 2H), 0.42-0.38 (m, 2H); ESI MS m/z 569 [C₃₁H₃₆N₈O₃+H]⁺; HPLC >99%, t_{R}=9.0 min.

### Example 72

### N-methyl-N′-{4-[1-methyl-4-(2-{[3-(1-pyrrolidinylmethyl)phenyl]amino}-4-pyrimidinyl)-1H-pyrazol-3-yl]phenyl}urea

Method A, 64% as yellow solid: mp 175-179° C.; ¹H NMR (500 MHz, DMSO-d₆) δ 10.65 (s, 1H), 9.81 (s, 1H), 8.91 (s, 1H), 8.34 (t, J=5.3 Hz, 2H), 7.79 (s, 1H), 7.60 (d, J=8.1 Hz, 1H), 7.42 (d, J=8.6 Hz, 2H), 7.36 (d, J=8.7 Hz, 2H), 7.27 (t, J=7.7 Hz, 1H), 7.20 (d, J=7.6 Hz, 1H), 6.69 (d, J=5.4 Hz, 1H), 4.22 (d, J=5.7 Hz, 2H), 3.94 (s, 3H), 3.36-3.34 (m, 2H), 3.05-3.01 (m, 2H), 2.65 (s, 3H), 2.04-1.97 (m, 2H), 1.88-1.86 (m, 2H); ESI MS m/z 483 [C₂₇H₃₀N₈O+H]⁺; HPLC >99%, t_{R}=8.9 min.

### Example 73

### N-[4-(4-{2-[(3-{[4-(2-hydroxyethyl)-1-piperazinyl]methyl}phenyl)amino]-4-pyrimidinyl}-1-methyl-1H-pyrazol-3-yl)phenyl]-N′-methylurea

Method A, 48% for 3 steps as a yellow solid: ¹H NMR (500 MHz, DMSO-d₆) δ 12.50-10.50 (m, 1H), 9.72 (bs, 1H), 8.84 (s, 1H), 8.33-8.31 (m, 2H), 7.79 (s, 1H), 7.66-7.64 (m, 1H), 7.43-7.42 (m, 2H), 7.35-7.34 (m, 2H), 7.29-7.26 (m, 1H), 7.21-7.19 (m, 1H), 6.66-6.65 (m, 1H), 6.20 (bs, 1 h), 3.94 (s, 3H), 3.75-3.22 (m, 15H), 2.70 (s, 3H), ESI MS m/z 542 [C₂₉H₃₅N₉O₂+H]⁺; HPLC 97.9% (AUC), t_{R}=8.38 min.

### Example 74

### N-(4-{4-[2-({3-[4-(2-hydroxyethyl)-1-piperazinyl]phenyl}amino)-4-pyrimidinyl]-1-methyl-1H-pyrazol-3-yl}phenyl)-1-pyrrolidinecarboxamide

¹H NMR (400 MHz, DMSO-d₆) δ ppm 9.97 (s, 1H), 9.41 (s, 1H), 8.29 (d, J=5.05 Hz, 1H), 8.24-8.23 (m, 1H), 7.56 (d, J=8.59 Hz, 2H), 7.53-7.50 (m, 1H), 7.37 (d, J=8.59 Hz, 2H), 7.24-7.21 (m, 1H), 7.10 (t, J=8.08 Hz, 1H), 6.62-6.54 (m, 1H), 6.57 (d, J=5.31 Hz, 1H), 5.41-5.40 (m, 1H), 3.95 (s, 3H), 3.83-3.78 (m, 2H), 3.73-3.66 (m, 2H), 3.64-3.58 (m, 2H), 3.43-3.34 (m, 4H), 3.28-3.16 (m, 4H), 3.09-3.05 (m, 2H), 1.91-1.84 (m, 4H); ESI MS (m/z) 568: LCMS retention time t_{R}=1.44 min: analytical HPLC t_{R}=2.03 min.

### Example 75

### N-{4-[1-methyl-4-(2-{[3-(4-methyl-1-piperazinyl)phenyl]amino}-4-pyrimidinyl)-1H-pyrazol-3-yl]phenyl}-1-pyrrolidinecarboxamide

¹H NMR (400 MHz, DMSO-d₆) δ ppm 10.44 (s, 1H) 9.52 (s, 1H) 8.30 (d, J=5.31 Hz, 1H), 8.28-8.23 (m, 1H), 7.57 (d, J=8.84 Hz, 2H), 7.47 (s, 1H), 7.37 (d, J=8.59 Hz, 2H), 7.22-7.18 (m, 1H), 7.11 (t, J=8.08 Hz, 1H), 6.63-6.59 (m, 1H), 6.59 (d, J=5.31 Hz, 1H), 3.95 (s, 3H), 3.72 (d, J=1.01 Hz, 2H), 3.54-3.48 (m, 2H), 3.40-3.36 (m, 4H), 3.21-3.10 (m, 2H), 3.05-2.98 (m, 2H), 2.83 (d, J=4.55 Hz, 3H), 1.88-1.83 (m, 4H); ESI MS (m/z) 538: LCMS retention time t_{R}=1.47 min: analytical HPLC t_{R}=2.06 min.

### Example 76

### (4-[2-({3-[2-(4-morpholinyl)ethyl]phenyl}amino)-4-pyrimidinyl]-3-{4-[(1-pyrrolidinylcarbonyl)amino]phenyl}-1H-pyrazol-1-yl)acetic acid

¹H NMR (400 MHz, acetone-d₆) δ ppm 11.48 (s, 1H), 8.60 (s, 1H), 8.39 (s, 2H), 7.93 (s, 1H), 7.77 (s, 1H), 7.60 (s, 4H), 7.32 (s, 2H), 7.02 (d, J=1.77 Hz, 1H), 5.19 (s, 2H), 4.12 (s, 4H), 3.75 (m, 2H), 3.44-3.52 (m, 4H), 3.30 (m, 2H), 3.00 (d, J=10.11 Hz, 2H), 1.96 (m, 4H); MS (ES) m/e 598 [M+H]⁺.

### Example 77

### {3-(4-{[(ethylamino)carbonyl]amino}phenyl)-4-[2-({3-[2-(4-morpholinyl)ethyl]phenyl}amino)-4-pyrimidinyl]-1H-pyrazol-1-yl}acetic acid

¹H NMR (400 MHz, acetone-d₆) δ ppm 10.79 (s, 2H), 8.46-8.57 (m, 1H), 8.31 (s, 1H), 7.76 (s, 1H), 7.67 (d, J=8.34 Hz, 1H), 7.45-7.56 (m, 3H), 7.39 (d, J=7.83 Hz, 1H), 7.22-7.33 (m, 1H), 7.00 (d, J=7.33 Hz, 1H), 6.91 (d, J=5.56 Hz, 1H), 5.15 (s, 2H), 4.04 (d, J=6.32 Hz, 4H), 3.86 (s, 2H), 3.70 (s, 2H), 3.37-3.46 (m, 2H), 3.31 (d, J=3.28 Hz, 2H), 3.25 (q, J=7.24 Hz, 4H), 3.09 (d, J=8.34 Hz, 2H), 3.06 (s, 2H), 1.13 (t, J=7.20 Hz, 3H); MS (ES) m/e 572 [M+H]⁺.

### Example 78

### N-ethyl-3-(4-{[(ethylamino)carbonyl]amino}phenyl)-4-[2-({3-[2-(4-morpholinyl)ethyl]phenyl}amino)-4-pyrimidinyl]-1H-pyrazole-1-carboxamide

¹H NMR (400 MHz, acetone-d₆) δ ppm 10.37 (s, 1H), 8.87 (s, 1H), 8.55 (s, 1H), 8.41 (d, J=5.31 Hz, 1H), 8.23 (t, J=5.81 Hz, 1H), 7.79 (s, 1H), 7.50-7.60 (m, 4H), 7.36 (d, J=7.83 Hz, 1H), 7.24 (t, J=7.83 Hz, 1H), 6.99 (d, J=5.56 Hz, 1H), 4.07-4.17 (m, 2H), 4.00 (t, J=11.75 Hz, 2H), 3.75 (d, J=11.37 Hz, 2H), 3.45-3.55 (m, 4H), 3.39 (s, 1H), 3.34 (s, 1H), 3.27 (q, J=7.07 Hz, 2H), 3.04-3.14 (m, 2H), 1.29 (t, J=7.20 Hz, 3H), 1.16 (t, 3H, J=7.2 Hz); MS (ES) m/e 585 [M+H]⁺.

### Example 79

### {3-(4-{[(dimethylamino)carbonyl]amino}phenyl)-4-[2-({3-[2-(4-morpholinyl)ethyl]phenyl}amino)-4-pyrimidinyl]-1H-pyrazol-1-yl}acetic acid

¹H NMR (400 MHz, acetone-d₆) δ ppm 8.60 (s, 1H), 8.38 (d, J=6.06 Hz, 1H), 7.87 (s, 1H), 7.78 (d, J=8.59 Hz, 1H), 7.55-7.63 (m, 4H), 7.33-7.40 (m, 2H), 7.29 (t, J=7.71 Hz, 1H), 6.99-7.07 (m, 2H), 5.20 (s, 2H), 4.11 (s, 2H), 4.02 (s, 2H), 3.76 (s, 2H), 3.55 (s, 2H), 3.39-3.50 (m, 4H), 3.23-3.34 (m, 2H), 3.06 (s, 6H); MS (ES) m/e 572 [M+H]⁺.

### Example 80

### N-(4-{1-ethyl-4-[2-({3-[2-(4-morpholinyl)ethyl]phenyl}amino)-4-pyrimidinyl]-1H-pyrazol-3-yl}-2-fluorophenyl)-1-pyrrolidinecarboxamide

¹H NMR (400 MHz, acetone-d₆) δ ppm 11.35 (s, 1H), 8.55 (s, 1H), 8.44 (s, 1H), 7.93 (d, J=8.08 Hz, 1H), 7.61 (s, 1H), 7.56 (dd, J=12.25, 1.89 Hz, 3H), 7.38 (s, 1H), 7.26 (t, J=7.83 Hz, 1H), 7.21 (s, 1H), 6.98 (s, 1H), 4.30-4.39 (m, 2H), 4.15 (s, 2H), 4.03 (s, 5H), 3.74 (s, 2H), 3.57 (d, J=2.78 Hz, 2H), 3.56 (s, 2H), 3.40-3.48 (m, 2H), 3.33 (s, 2H), 2.95 (s, 2H), 2.01 (d, J=2.53 Hz, 4H), 1.51-1.61 (t, J=4.40 Hz, 3H); MS (ES) m/e 585 [M+H]⁺.

### Example 81

### N-(4-{1-ethyl-4-[2-({3-[2-(4-morpholinyl)ethyl]phenyl}amino)-4-pyrimidinyl]-1H-pyrazol-5-yl}-2-fluorophenyl)-1-pyrrolidinecarboxamide

¹H NMR (400 MHz, acetone-d₆) δ ppm 11.58 (s, 1H), 8.37 (td, J=8.34, 2.53 Hz, 1H), 8.29 (s, 1H), 8.27 (d, J=6.32 Hz, 1H), 7.54 (d, J=2.27 Hz, 1H), 7.52 (s, 1H), 7.33-7.41 (m, 1H), 7.32 (d, J=1.77 Hz, 1H), 7.23-7.29 (m, 2H), 7.13 (d, J=7.58 Hz, 1H), 6.72 (d, J=6.4 Hz, 2H), 4.09 (q, J=7.16 Hz, 4H), 3.95 (t, J=12.13 Hz, 2H), 3.76 (d, J=11.87 Hz, 2H), 3.52-3.61 (m, 6H), 3.32 (t, J=10.61 Hz, 2H), 3.22-3.28 (m, 2H), 1.96-2.04 (m, 4H), 1.36 (t, J=7.20 Hz, 3H); MS (ES) m/e 585 [M+H]⁺.

General Synthesis of Amide Targets from Intermediate 33.

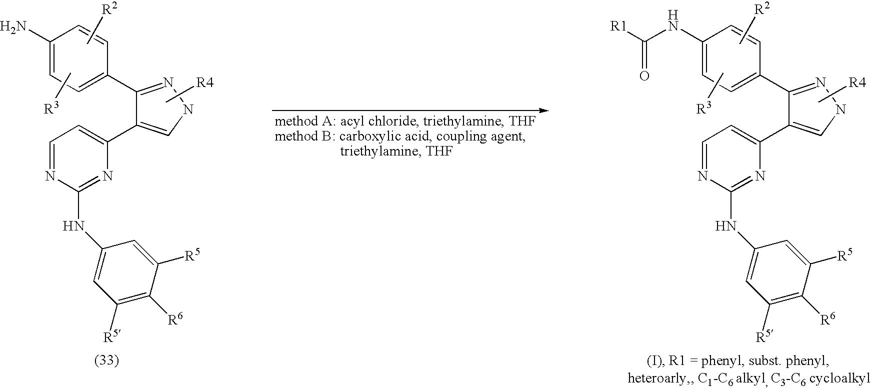

### Example 82

### N-[4-(4-{2-[(3-{[4-(2-hydroxyethyl)-1-piperazinyl]methyl}phenyl)amino]-4-pyrimidinyl}-1-methyl-1H-pyrazol-3-yl)phenyl]-2,2-dimethylpropanamide

33% as a yellow solid: ¹H NMR (500 MHz, DMSO-d₆) δ 12.05-10.45 (m, 1H), 9.62 (bs, 1H), 9.29 (s, 1H), 8.32-8.30 (m, 2H), 7.71-7.70 (m, 1H), 7.69-7.68 (m, 2H), 7.65-7.64 (m, 1H), 7.43-7.42 (m, 2H), 7.25-7.13 (m, 2H), 6.63-6.62 (m, 1H), 3.95-3.92 (m, 5H), 3.81-2.90 (m, 13H), 1.24 (s, 9H); ESI MS m/z 569 [C₃₂H₄₁N₈O₂+H]⁺; HPLC >99.0% (AUC), t_{R}=9.65 min.

### Example 83

### 2,2-dimethyl-N-{4-[1-methyl-4-(2-{[3-(1-pyrrolidinylmethyl)phenyl]amino}-4-pyrimidinyl)-1H-pyrazol-3-yl]phenyl}propanamide

65% as a yellow powder: mp 218-223° C.; ¹H NMR (500 MHz, DMSO-d₆) δ 10.45 (s, 1H), 9.71 (s, 1H), 9.30 (s, 1H), 8.32 (t, J=2.8 Hz, 2H), 7.82 (s, 1H), 7.70 (d, J=8.4 Hz, 2H), 7.62 (d, J=8.1 Hz, 1H), 7.43 (d, J=8.6 Hz, 2H), 7.26 (t, J=7.8 Hz, 1H), 7.14 (d, J=7.6 Hz, 1H), 6.65 (d, J=5.2 Hz, 1H), 4.23-4.21 (m, 2H), 3.95 (s, 3H), 3.36-3.35 (m, 2H), 3.06-3.02 (m, 2H), 2.02-1.99 (m, 2H), 1.88-1.85 (m, 2H), 1.24 (s, 9H); ESI MS m/z 510 [C₃₀H₃₅N₇O+H]⁺; HPLC 98.1% (AUC), t_{R}=10.5 min.

### Example 84

### N-(4-{4-[2-{(3-fluorophenyl)amino]-4-pyrimidinyl}-1-[2-(4-morpholinyl)ethyl]-1H-pyrazol-3-yl}phenyl)-1-pyrrolidinecarboxamide

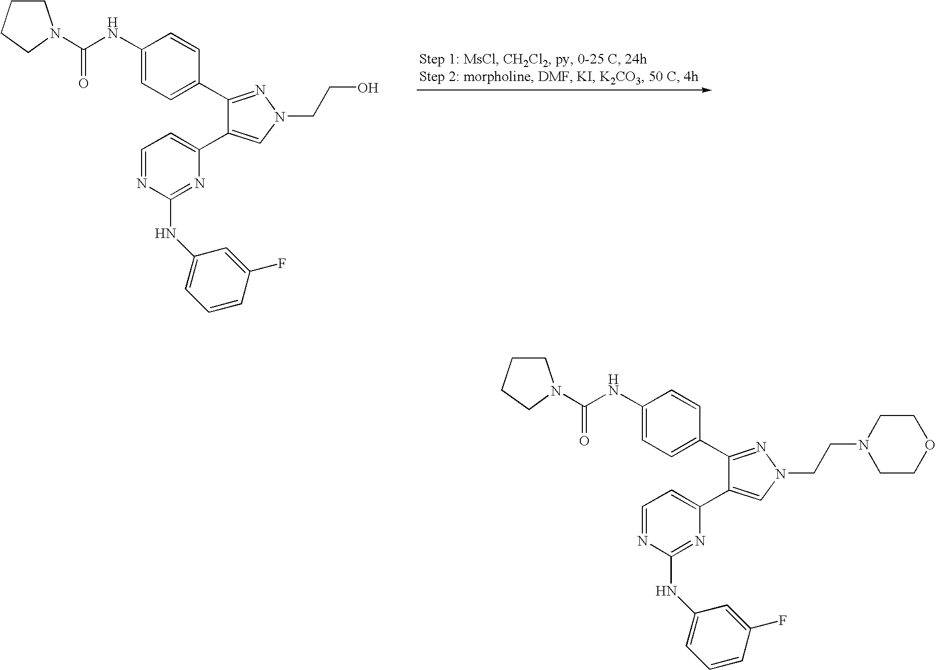

Step 1: To a solution of the pyrazole (40 mg, 82 μmol) in 3:1 CH₂Cl₂-pyridine (500 μL) at 0° C. was added methanesulfonyl chloride (10 μL, 100 μmol). The reaction was stirred at room temperature for 1.5 h. Analysis of the reaction mixture by LC-MS indicated the formation of the desired intermediate mesylate along with unreacted starting material. The reaction was cooled to 0° C. and additional methanesulfonyl chloride (10 μL, 100 μmol) was added and the reaction was stirred at room temperature overnight. LC-MS analysis of the reaction mixture indicated complete conversion of starting material.

Step 2: The reaction mixture from Step 1 was added dropwise to a solution of morpholine (500 μL, 5.7 mmol) in DMF (10 mL) containing potassium iodide (100 mg, 0.6 mmol) and potassium carbonate (1 g, 7.2 mmol) and heated at 50° C. for 4 h. The reaction was cooled to room temperature, poured into water (200 mL) and extracted with ethyl acetate (4×50 mL). The combined organic phases were washed with 5% lithium chloride solution (50 mL) and brine (50 mL), dried over sodium sulfate and purified by chromatography (silica gel, 0-10% MeOH/CH₂Cl₂ containing 2% NH₄OH) to afford Example 76 (20 mg, 44%) as a white solid. mp 167-168° C.; ¹H NMR (500 MHz, DMSO-d₆) δ 9.71 (s, 1H), 8.33-8.32 (m, 1H), 8.29 (m, 1H), 8.18 (m, 1H), 7.68-7.65 (m, 1H), 7.57-7.56 (m, 2H), 7.46-7.44 (m, 1H), 7.36-7.34 (m 2H), 7.21-7.19 (m, 1H), 6.70-6.67 (m, 1H), 6.64-6.63 (m, 1H), 4.32 (m, 2H), 3.57 (m, 4H), 3.37 (m, 4H), 2.79-2.77 (m, 2H), 2.50-2.46 (m, 4H), 1.85 (m, 4H); ESI MS m/z 557 [C₃₀H₃₃FN₈O₂+H]⁺; HPLC >99% (AUC), t_{R}=10.66 min.

1. In a first aspect, the present invention is a compound of formula (I): 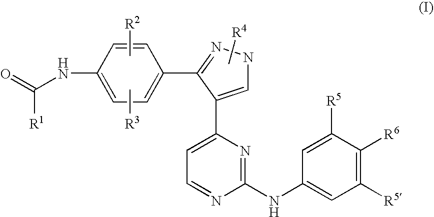
or a pharmaceutically acceptable salt thereof, or a solvate thereof, or a combination thereof, wherein:
R¹ represents phenyl, substituted-phenyl, heteroaryl, C₁-C₆ alkyl, C₃-C₆ cycloalkyl, or —NR⁷R⁸;
R² and R³ each independently represent H, halo, C₁-C₃ alkyl, or —O—C₁-C₃ alkyl;
R⁴, a substituent for one of the nitrogen atoms of the pyrazole ring, represents H, C₁-C₆ alkyl, substituted C₁-C₆ alkyl, C₃-C₆ cycloalkyl, —C(O)C₁-C₆ alkyl, —C(O)-substituted C₁-C₆ alkyl, —C(O)NR⁷R⁸, —S(O)₂—C₁-C₆ alkyl, —S(O)₂—C₃-C₆ cycloalkyl, or —C(O)NH—C₁-C₆ alkyl;
R⁵, R^{5′}, and R⁶ each independently represent H, halo, C₁-C₆ alkyl, substituted C₁-C₆ alkyl, —NH—C(O)-substituted C₁-C₆ alkyl, —O—C₁-C₆ alkyl, —O-substituted C₁-C₆ alkyl, —NR⁷R⁸, or hydroxyl; and
R⁷ and R⁸ each independently represent H, C₁-C₆ alkyl, substituted C₁-C₆ alkyl, C₃-C₆ cycloalkyl, phenyl, substituted phenyl or heteroaryl, or form, together with the nitrogen atom to which they are attached, a substituent selected from the group consisting of pyrrolidinyl, piperidinyl, morpholinyl, piperazinyl, 4-(C₁-C₆ alkyl)-piperazin-1-yl, and 4-(hydroxy-C₂-C₆ alkyl)-piperazin-1-yl.

2. The compound of claim 1, wherein R¹ represents cyclopropyl, phenyl, t-butyl, —NHCH₃, —NHCH₂CH₃, —NHCH₂CH₂CH₃, —NH-cyclopropyl, —N(CH₃)₂, —N(CH₂CH₃)₂, or piperidinyl; R² and R³ each independently represent H or F; R⁴ represents methyl, ethyl, isopropyl, isobutyl, methoxyethyl, hydroxyethyl, hydroxypropyl, dihydroxypropyl, morpholinylethyl, 2,2,2-trifluoroethyl, ethylaminocarbonyl, mesyl, piperidinylcarbonylmethyl, or carboxymethyl; R^{5′} and R⁶ are H; and R⁵ represents F, (dimethylamino)methylcarbonylamino, —(CH₂)ₙ-morpholinyl, —(CH₂)ₙ-piperidinyl, —(CH₂)ₙ-[4-(C₁-C₆ alkyl)-piperazin-1-yl], or —(CH₂)ₙ-[4-(hydroxy-C₁-C₆ alkyl)-piperazin-1-yl], where n is an integer from 0 to 6.

3. The compound of claim 1 which has an IC₅₀ of less than 10 μM against Aurora A or Aurora B or both.

4. A composition comprising (a) the compound of claim 1 or a pharmaceutically acceptable salt thereof, or a solvate thereof, or a combination thereof; and (b) one or more pharmaceutically acceptable diluents, carriers, or excipients.

5. A method for treating a disease of cell proliferation comprising administering to a patient in need thereof the compound of claim 1, or a pharmaceutically acceptable salt thereof, or a solvate thereof, or a combination thereof.

6. A compound selected from the group consisting of:
N-(4-{4-[2-({3-[2-(4-Morpholinyl)ethyl]phenyl}amino)-4-pyrimidinyl]-1H-pyrazol-3-yl}phenyl)benzamide;
N-(4-{1-Methyl-4-[2-({3-[2-(4-morpholinyl)ethyl]phenyl}amino)-4-pyrimidinyl]-1H-pyrazol-5-yl}phenyl)benzamide;
N-(4-{1-Methyl-4-[2-({3-[2-(4-morpholinyl)ethyl]phenyl}amino)-4-pyrimidinyl]-1H-pyrazol-3-yl}phenyl)benzamide;
N-[4-(4-{2-[(3-Fluorophenyl)amino]-4-pyrimidinyl}-1H-pyrazol-3-yl)phenyl]benzamide;
N-{4-[4-(2-{[3-(4-Methyl-1-piperazinyl)phenyl]amino}-4-pyrimidinyl)-1H-pyrazol-3-yl]phenyl}benzamide;
N-(4-{4-[2-({3-[(N,N-Dimethylglycyl)amino]phenyl}amino)-4-pyrimidinyl]-1H-pyrazol-3-yl}phenyl)benzamide;
N-{4-[4-(2-{[3-(4-Morpholinylmethyl)phenyl]amino}-4-pyrimidinyl)-1H-pyrazol-3-yl]phenyl}benzamide;
N-{4-[4-(2-{[3-(4-Methyl-1-piperazinyl)phenyl]amino}-4-pyrimidinyl)-1H-pyrazol-3-yl]phenyl}cyclopropanecarboxamide;
N-(4-{4-[2-({3-[2-(4-Morpholinyl)ethyl]phenyl}amino)-4-pyrimidinyl]-1H-pyrazol-3-yl}phenyl)cyclopropanecarboxamide;
N-(4-{1-Methyl-4-[2-({3-[2-(4-morpholinyl)ethyl]phenyl}amino)-4-pyrimidinyl]-1H-pyrazol-5-yl}phenyl)cyclopropanecarboxamide;
N-(4-{1-Methyl-4-[2-({3-[2-(4-morpholinyl)ethyl]phenyl}amino)-4-pyrimidinyl]-1H-pyrazol-3-yl}phenyl)cyclopropanecarboxamide;
N-{4-[1-Methyl-4-(2-{[3-(4-methylpiperazin-1-yl)phenyl]amino}pyrimidin-4-yl)-1H-pyrazol-5-yl]phenyl}benzamide;
N-{4-[1-Methyl-4-(2-{[3-(4-methylpiperazin-1-yl)phenyl]amino}pyrimidin-4-yl)-1H-pyrazol-3-yl]phenyl}benzamide;
N-{4-[1-Methyl-4-(2-{[3-(4-methylpiperazin-1-yl)phenyl]amino}pyrimidin-4-yl)-1H-pyrazol-5-yl]phenyl}cyclopropanecarboxamide;
N-{4-[1-Methyl-4-(2-{[3-(4-methylpiperazin-1-yl)phenyl]amino}pyrimidin-4-yl)-1H-pyrazol-3-yl]phenyl}cyclopropanecarboxamide;
N-{4-[1-Ethyl-4-(2-{[3-(4-methyl-1-piperazinyl)phenyl]amino}-4-pyrimidinyl)-1H-pyrazol-5-yl]phenyl}cyclopropanecarboxamide;
N-{4-[1-Ethyl-4-(2-{[3-(4-methylpiperazin-1-yl)phenyl]amino}pyrimidin-4-yl)-1H-pyrazol-3-yl]phenyl}cyclopropanecarboxamide;
N-(4-{4-[2-({3-[2-(4-Morpholinyl)ethyl]phenyl}amino)-4-pyrimidinyl]-1H-pyrazol-3-yl}phenyl)-1-pyrrolidinecarboxamide;
N-{4-[4-(2-{[3-(4-Methyl-1-piperazinyl)phenyl]amino}-4-pyrimidinyl)-1H-pyrazol-3-yl]phenyl}-1-pyrrolidinecarboxamide;
N-(4-{1-Ethyl-4-[2-({3-[2-(4-morpholinyl)ethyl]phenyl}amino)-4-pyrimidinyl]-1H-pyrazol-5-yl}phenyl)-1-pyrrolidinecarboxamide;
N-(4-{1-Ethyl-4-[2-({3-[2-(4-morpholinyl)ethyl]phenyl}amino)-4-pyrimidinyl]-1H-pyrazol-3-yl}phenyl)-1-pyrrolidinecarboxamide;
N-ethyl-N′-(4-{1-ethyl-4-[2-({3-[2-(4-morpholinyl)ethyl]phenyl}amino)-4-pyrimidinyl]-1H-pyrazol-5-yl}phenyl)urea;
N-ethyl-N′-(4-{1-ethyl-4-[2-({3-[2-(4-morpholinyl)ethyl]phenyl}amino)-4-pyrimidinyl]-1H-pyrazol-3-yl}phenyl)urea;
N-propyl-N′-(4-{1-ethyl-4-[2-({3-[2-(4-morpholinyl)ethyl]phenyl}amino)-4-pyrimidinyl]-1H-pyrazol-5-yl}phenyl)urea
N-propyl-N′-(4-{1-ethyl-4-[2-({3-[2-(4-morpholinyl)ethyl]phenyl}amino)-4-pyrimidinyl]-1H-pyrazol-3-yl}phenyl)urea;
N-cyclopropyl-N′-(4-{1-ethyl-4-[2-({3-[2-(4-morpholinyl)ethyl]phenyl}amino)-4-pyrimidinyl]-1H-pyrazol-5-yl}phenyl)urea;
N-cyclopropyl-N′-(4-{1-ethyl-4-[2-({3-[2-(4-morpholinyl)ethyl]phenyl}amino)-4-pyrimidinyl]-1H-pyrazol-3-yl}phenyl)urea;
N-(4-{1-(1-methylethyl)-4-[2-({3-[2-(4-morpholinyl)ethyl]phenyl}amino)-4-pyrimidinyl]-1H-pyrazol-3-yl}phenyl)cyclopropanecarboxamide;
N-(4-{1-(1-methylethyl)-4-[2-({3-[2-(4-morpholinyl)ethyl]phenyl}amino)-4-pyrimidinyl]-1H-pyrazol-5-yl}phenyl)cyclopropanecarboxamide;
N-(4-{1-ethyl-4-[2-({3-[2-(4-morpholinyl)ethyl]phenyl}amino)-4-pyrimidinyl]-1H-pyrazol-3-yl}phenyl)cyclopropanecarboxamide;
N-(4-{1-ethyl-4-[2-({3-[2-(4-morpholinyl)ethyl]phenyl}amino)-4-pyrimidinyl]-1H-pyrazol-5-yl}phenyl)cyclopropanecarboxamide;
N-(4-{1-(2-hydroxyethyl)-4-[2-({3-[2-(4-morpholinyl)ethyl]phenyl}amino)-4-pyrimidinyl]-1H-pyrazol-3-yl}phenyl)cyclopropanecarboxamide;
N-(4-{1-[2-(methyloxy)ethyl]-4-[2-({3-[2-(4-morpholinyl)ethyl]phenyl}amino)-4-pyrimidinyl]-1H-pyrazol-3-yl}phenyl)cyclopropanecarboxamide;
N-(4-{1-[2-(methyloxy)ethyl]-4-[2-({3-[2-(4-morpholinyl)ethyl]phenyl}amino)-4-pyrimidinyl]-1H-pyrazol-5-yl}phenyl)cyclopropanecarboxamide;
N-(4-{1-(2-methylpropyl)-4-[2-({3-[2-(4-morpholinyl)ethyl]phenyl}amino)-4-pyrimidinyl]-1H-pyrazol-3-yl}phenyl)cyclopropanecarboxamide;
N-(4-{1-(2-methylpropyl)-4-[2-({3-[2-(4-morpholinyl)ethyl]phenyl}amino)-4-pyrimidinyl]-1H-pyrazol-5-yl}phenyl)cyclopropanecarboxamide;
N-(4-{1-(methylsulfonyl)-4-[2-({3-[2-(4-morpholinyl)ethyl]phenyl}amino)-4-pyrimidinyl]-1H-pyrazol-3-yl}phenyl)cyclopropanecarboxamide;
N-(4-{1-(2-hydroxyethyl)-4-[2-({3-[2-(4-morpholinyl)ethyl]phenyl}amino)-4-pyrimidinyl]-1H-pyrazol-5-yl}phenyl)cyclopropanecarboxamide;
N-(4-{4-[2-({3-[2-(4-morpholinyl)ethyl]phenyl}amino)-4-pyrimidinyl]-1-[2-oxo-2-(1-pyrrolidinyl)ethyl]-1H-pyrazol-3-yl}phenyl)cyclopropanecarboxamide;
N-{4-[4-[2-({3-[2-(4-morpholinyl)ethyl]phenyl}amino)-4-pyrimidinyl]-1-(2,2,2-trifluoroethyl)-1H-pyrazol-3-yl]phenyl}cyclopropanecarboxamide;
3-{4-[(cyclopropylcarbonyl)amino]phenyl}-N-ethyl-4-[2-({3-[2-(4-morpholinyl)ethyl]phenyl}amino)-4-pyrimidinyl]-1H-pyrazole-1-carboxamide;
N-(4-{1-(3-hydroxypropyl)-4-[2-({3-[2-(4-morpholinyl)ethyl]phenyl}amino)-4-pyrimidinyl]-1H-pyrazol-3-yl}phenyl)cyclopropanecarboxamide;
N-(4-{1-(3-hydroxypropyl)-4-[2-({3-[2-(4-morpholinyl)ethyl]phenyl}amino)-4-pyrimidinyl]-1H-pyrazol-5-yl}phenyl)cyclopropanecarboxamide;
N-(4-{1-[(2S)-2,3-dihydroxypropyl]-4-[2-({3-[2-(4-morpholinyl)ethyl]phenyl}amino)-4-pyrimidinyl]-1H-pyrazol-3-yl}phenyl)cyclopropanecarboxamide;
N-(4-{1-[(2R)-2,3-dihydroxypropyl]-4-[2-({3-[2-(4-morpholinyl)ethyl]phenyl}amino)-4-pyrimidinyl]-1H-pyrazol-3-yl}phenyl)cyclopropanecarboxamide;
N-(4-{1-(3-hydroxypropyl)-4-[2-({3-[2-(4-morpholinyl)ethyl]phenyl}amino)-4-pyrimidinyl]-1H-pyrazol-3-yl}phenyl)-1-pyrrolidinecarboxamide;
N-(4-{1-[(2R)-2,3-dihydroxypropyl]-4-[2-({3-[2-(4-morpholinyl)ethyl]phenyl}amino)-4-pyrimidinyl]-1H-pyrazol-3-yl}phenyl)-1-pyrrolidinecarboxamide;
N-(4-{1-[(2S)-2,3-dihydroxypropyl]-4-[2-({3-[2-(4-morpholinyl)ethyl]phenyl}amino)-4-pyrimidinyl]-1H-pyrazol-3-yl}phenyl)-1-pyrrolidinecarboxamide;
N,N-diethyl-N′-{4-[1-(2-hydroxyethyl)-4-(2-{[3-(4-methyl-1-piperazinyl)phenyl]amino}-4-pyrimidinyl)-1H-pyrazol-3-yl]phenyl}urea;
N′-{4-[1-(2-hydroxyethyl)-4-(2-{[3-(1-pyrrolidinylmethyl)phenyl]amino}-4-pyrimidinyl)-1H-pyrazol-3-yl]phenyl}-N,N-dimethylurea;
N′-(4-{1-ethyl-4-[2-({3-[2-(4-morpholinyl)ethyl]phenyl}amino)-4-pyrimidinyl]-1H-pyrazol-3-yl}phenyl)-N,N-dimethylurea;
N,N-diethyl-N′-{4-[1-methyl-4-(2-{[3-(1-pyrrolidinylmethyl)phenyl]amino}-4-pyrimidinyl)-1H-pyrazol-3-yl]phenyl}urea;
N,N-dimethyl-N′-{4-[1-methyl-4-(2-{[3-(1-pyrrolidinylmethyl)phenyl]amino}-4-pyrimidinyl)-1H-pyrazol-3-yl]phenyl}urea;
N-{4-[4-{2-[(3-fluorophenyl)amino]-4-pyrimidinyl}-1-(2-hydroxyethyl)-1H-pyrazol-3-yl]phenyl}-1-pyrrolidinecarboxamide;
N-cyclopropyl-N′-(4-{1-methyl-4-[2-({3-[2-(4-morpholinyl)ethyl]phenyl}amino)-4-pyrimidinyl]-1H-pyrazol-3-yl}phenyl)urea;
N-(4-{1-methyl-4-[2-({3-[2-(4-morpholinyl)ethyl]phenyl}amino)-4-pyrimidinyl]-1H-pyrazol-5-yl}phenyl)-1-pyrrolidinecarboxamide;
N-(4-{1-(2-hydroxyethyl)-4-[2-({3-[2-(4-morpholinyl)ethyl]phenyl}amino)-4-pyrimidinyl]-1H-pyrazol-3-yl}phenyl)-1-pyrrolidinecarboxamide;
N-cyclopropyl-N′-(4-{1-(2-hydroxyethyl)-4-[2-({3-[2-(4-morpholinyl)ethyl]phenyl}amino)-4-pyrimidinyl]-1H-pyrazol-3-yl}-2-methylphenyl)urea;
N,N-diethyl-N′-(4-{4-[2-({3-[4-(2-hydroxyethyl)-1-piperazinyl]phenyl}amino)-4-pyrimidinyl]-1-methyl-1H-pyrazol-3-yl}phenyl)urea;
N,N-diethyl-N′-{4-[1-methyl-4-(2-{[3-(4-methyl-1-piperazinyl)phenyl]amino}-4-pyrimidinyl)-1H-pyrazol-3-yl]phenyl}urea;
N-ethyl-N′-(4-{1-methyl-4-[2-({3-[2-(4-morpholinyl)ethyl]phenyl}amino)-4-pyrimidinyl]-1H-pyrazol-3-yl}phenyl)urea;
N,N-diethyl-N′-[4-(4-{2-[(3-{[4-(2-hydroxyethyl)-1-piperazinyl]methyl}phenyl)amino]-4-pyrimidinyl}-1-methyl-1H-pyrazol-3-yl)phenyl]urea;
N-(4-{1-methyl-4-[2-({3-[2-(4-morpholinyl)ethyl]phenyl}amino)-4-pyrimidinyl]-1H-pyrazol-3-yl}phenyl)-1-pyrrolidinecarboxamide;
N-ethyl-N′-(4-{1-methyl-4-[2-({3-[2-(4-morpholinyl)ethyl]phenyl}amino)-4-pyrimidinyl]-1H-pyrazol-3-yl}phenyl)urea;
N,N-dimethyl-N′-(4-{1-methyl-4-[2-({3-[2-(4-morpholinyl)ethyl]phenyl}amino)-4-pyrimidinyl]-1H-pyrazol-3-yl}phenyl)urea;
N-ethyl-N′-(4-{1-(2-hydroxyethyl)-4-[2-({3-[2-(4-morpholinyl)ethyl]phenyl}amino)-4-pyrimidinyl]-1H-pyrazol-3-yl}-2-methylphenyl)urea;
N,N-diethyl-N′-(4-{1-(2-hydroxyethyl)-4-[2-({3-[2-(4-morpholinyl)ethyl]phenyl}amino)-4-pyrimidinyl]-1H-pyrazol-3-yl}phenyl)urea;
N,N-diethyl-N′-{4-[1-(2-hydroxyethyl)-4-(2-{[3-(4-methyl-1-piperazinyl)phenyl]amino}-4-pyrimidinyl)-1H-pyrazol-3-yl]phenyl}urea;
N,N-diethyl-N′-(4-{1-(2-hydroxyethyl)-4-[2-({3-[(4-methyl-1-piperazinyl)methyl]phenyl}amino)-4-pyrimidinyl]-1H-pyrazol-3-yl}phenyl)urea;
N-cyclopropyl-N′-[4-{1-(2-hydroxyethyl)-4-[2-({3-[2-(4-morpholinyl)ethyl]phenyl}amino)-4-pyrimidinyl]-1H-pyrazol-3-yl}-2-(methyloxy)phenyl]urea;
N-cyclopropyl-N′-(4-{1-(2-hydroxyethyl)-4-[2-({3-[2-(4-morpholinyl)ethyl]phenyl}amino)-4-pyrimidinyl]-1H-pyrazol-3-yl}phenyl)urea;
N-methyl-N′-{4-[1-methyl-4-(2-{[3-(1-pyrrolidinylmethyl)phenyl]amino}-4-pyrimidinyl)-1H-pyrazol-3-yl]phenyl}urea;
N-[4-(4-{2-[(3-{[4-(2-hydroxyethyl)-1-piperazinyl]methyl}phenyl)amino]-4-pyrimidinyl}-1-methyl-1H-pyrazol-3-yl)phenyl]-N′-methylurea;
N-(4-{4-[2-({3-[4-(2-hydroxyethyl)-1-piperazinyl]phenyl}amino)-4-pyrimidinyl]-1-methyl-1H-pyrazol-3-yl}phenyl)-1-pyrrolidinecarboxamide;
N-{4-[1-methyl-4-(2-{[3-(4-methyl-1-piperazinyl)phenyl]amino}-4-pyrimidinyl)-1H-pyrazol-3-yl]phenyl}-1-pyrrolidinecarboxamide;
(4-[2-({3-[2-(4-morpholinyl)ethyl]phenyl}amino)-4-pyrimidinyl]-3-{4-[(1-pyrrolidinylcarbonyl)amino]phenyl}-1H-pyrazol-1-yl)acetic acid;
{3-(4-{[(ethylamino)carbonyl]amino}phenyl)-4-[2-({3-[2-(4-morpholinyl)ethyl]phenyl}amino)-4-pyrimidinyl]-1H-pyrazol-1-yl}acetic acid;
N-ethyl-3-(4-{[(ethylamino)carbonyl]amino}phenyl)-4-[2-({3-[2-(4-morpholinyl)ethyl]phenyl}amino)-4-pyrimidinyl]-1H-pyrazole-1-carboxamide;
{3-(4-{[(dimethylamino)carbonyl]amino}phenyl)-4-[2-({3-[2-(4-morpholinyl)ethyl]phenyl}amino)-4-pyrimidinyl]-1H-pyrazol-1-yl}acetic acid;
N-(4-{1-ethyl-4-[2-({3-[2-(4-morpholinyl)ethyl]phenyl}amino)-4-pyrimidinyl]-1H-pyrazol-3-yl}-2-fluorophenyl)-1-pyrrolidinecarboxamide;
N-(4-{1-ethyl-4-[2-({3-[2-(4-morpholinyl)ethyl]phenyl}amino)-4-pyrimidinyl]-1H-pyrazol-5-yl}-2-fluorophenyl)-1-pyrrolidinecarboxamide;
N-[4-(4-{2-[(3-{[4-(2-hydroxyethyl)-1-piperazinyl]methyl}phenyl)amino]-4-pyrimidinyl}-1-methyl-1H-pyrazol-3-yl)phenyl]-2,2-dimethylpropanamide;
2,2-dimethyl-N-{4-[1-methyl-4-(2-{[3-(1-pyrrolidinylmethyl)phenyl]amino}-4-pyrimidinyl)-1H-pyrazol-3-yl]phenyl}propanamide; and
N-(4-{4-{2-[(3-fluorophenyl)amino]-4-pyrimidinyl}-1-[2-(4-morpholinyl)ethyl]-1H-pyrazol-3-yl}phenyl)-1-pyrrolidinecarboxamide;
or a pharmaceutically acceptable salt thereof, or a solvate thereof, or a combination thereof.

7. The compound of claim 6 which is N,N-diethyl-N′-(4-{4-[2-({3-[4-(2-hydroxyethyl)-1-piperazinyl]phenyl}amino)-4-pyrimidinyl]-1-methyl-1H-pyrazol-3-yl}phenyl)urea, or a pharmaceutically acceptable salt thereof, or a solvate thereof, or a combination thereof.

8. The compound of claim 6 which is N,N-diethyl-N′-{4-[1-methyl-4-(2-{[3-(4-methyl-1-piperazinyl)phenyl]amino}-4-pyrimidinyl)-1H-pyrazol-3-yl]phenyl}urea, or a pharmaceutically acceptable salt thereof, or a solvate thereof, or a combination thereof.

9. The compound of claim 6 which is N′-{4-[1-(2-hydroxyethyl)-4-(2-{[3-(1-pyrrolidinylmethyl)phenyl]amino}-4-pyrimidinyl)-1H-pyrazol-3-yl]phenyl}-N,N-dimethylurea, or a pharmaceutically acceptable salt thereof, or a solvate thereof, or a combination thereof.
